(19) United States
(12) Patent Application Publication
(10) Pub. No.: US 2015/0111836 A1
(43) Pub. Date: Apr. 23, 2015

(54) CELLULAR APOBEC3 PROTEINS AND MODULATORS THEREOF FOR REGULATING DNA REPAIR PROCESSES AND TREATING PROLIFERATIVE DISEASES

(71) Applicant: YISSUM RESEARCH DEVELOPMENT COMPANY OF THE HEBREW UNIVERSITY OF JERUSALEM LTD., Jerusalem (IL)

(72) Inventors: Moshe Kotler, Mevasseret Zion (IL); Roni Nowarski, Jerusalem (IL); Elena Britan-Rosich, Jerusalem (IL); Lea Baraz, Jerusalem (IL)

(21) Appl. No.: 14/378,199

(22) PCT Filed: Feb. 12, 2013
(86) PCT No.: PCT/IL2013/050130

§ 371 Date: Aug. 12, 2014

Related U.S. Application Data
(60) Provisional application No. 61/728,294, filed on Nov. 20, 2012.
(60) Provisional application No. 61/597,793, filed on Feb. 12, 2012.

(51) Int. Cl. C07K 14/005; C12Q 1/68

(52) U.S. Cl. CPC C07K 14/005; C12Q 1/6883; C12Q 2600/136; C12Q 2600/158; C12N 2740/16033
USPC 514/21.5; 530/326; 435/6.13

(57) ABSTRACT

The present invention provides methods, compositions and kits for modulating DSB repair processes in a subject in need thereof. More specifically, the invention provides the use of compounds that modulate the expression or activity of at least one APOBEC family member for modulating DSB repair processes.

### FIELD OF THE INVENTION

The invention relates to regulation of DSB (double strand brakes) repair processes. More particularly, the invention provides compositions and methods modulating DNA repair processes for the treatment of proliferative disorders and DSB (double strand brakes) associated disorders.

### LIST OR REFERENCES

1. Bennett, R. P. et al., J. Biol. Chem. 283:33329-33336 (2008);
2. Huthoff, H. et al., PLoS Pathog. 5:e1000330 (2009);
3. Shandilya, S. M. et al., Structure 18:28-38 (2010);
4. Zhou, B. B. and Elledge, S. J., Nature 408(6811):433-439 (2000);
5. Chelico, L. et al., Nat. Struct. Mol. Biol. 13:392-399 (2006);
6. Stopak, K. et al., Mol Cell. 12(3): 591-601 (2003);
7. Diggle, C. P. et al., Nucleic Acids Res. 31(15):e83 (2003);
8. Nowarski, R., et al., Nat. Struct. Mol. Biol. 15(10):1059-1066 (2008);
9. Opi, S. et al., J. Virol. 81:8236-46 (2007);
10. Shahar, O. D. et al. Oncogene. doi:10.1038onc.516 (2011);
11. Pierce, A. J. Genes Dev. 13(20):2633-2638 (1999);
12. Wold, M. S., Annu. Rev. Biochem. 66(61-92) (1997).

### BACKGROUND OF THE INVENTION

Throughout this application, various publications, including United States patents, are referenced by author and year and patents by number. The disclosures of these publications and patents and patent applications in their entireties are hereby incorporated by reference into this application in order to more fully describe the state of the art to which this invention pertains.

APOBEC3 (A3G) proteins catalyze deamination of cytidines in ssDNA, providing innate protection against retroviral replication and retrotransposition. The native form of A3G is multimer. A3G multimers consist of dimeric subunits are suggested to engage in protein-protein interactions, or protein-RNA interactions. A3G contains two zinc-coordinating (Z) motifs which bind ssDNA with similar affinity, a C-terminal catalytic domain (CTD) and an N-terminal pseudo-catalytic domain (NTD). Both the CTD and NTD were independently implicated in mediating A3G oligomerization [1-3]. The inventors have previously shown that the catalytically active form of A3G is monomeric, and suggested that A3G multimers undergo disassembly to dimers and monomers upon interaction with ssDNA. Seven APOBEC3 (A3) genes, designated A3A, B, C, DE, F, G and H, are encoded within a single chromosomal cluster. The deamination of cytidine is catalyzed by A3 proteins through a catalytic domain containing the conserved zinc-coordinating motif (HC)XE(X)23-28CXXC. A3G, as well as A3B and A3F, contains two zinc-coordinating motifs which contribute unequally to the biological functions of this enzyme.

DNA DSBs are highly genotoxic lesions, constituting the primary damage induced by ionizing radiation (IR) and specific anti-tumor drugs. The two major DSB repair pathways are non-homologous end-joining (NHEJ), in which broken DNA ends are directly processed and ligated without the requirement for extensive sequence homology between the DNA ends, and homologous recombination (HR), which depends on a homologous chromatid or chromosome as a template for repair. DSB repair via both mechanisms is initiated by sensory proteins, mainly the Mre11-Rad50-Nbs1 (MRN) and the DNA-dependent protein kinase (DNA-PK) complexes, which bind directly to broken DNA ends in a cell-cycle-dependent manner [4]. These complexes were shown to mediate DNA end-synapsis, an initial stage of bringing two broken DNA termini at close proximity required for further processing, joining and cell cycle checkpoint signaling. Still, the mechanism whereby DNA end-synapsis occurs is not fully understood and may involve the function of yet unidentified proteins [4].

Ionizing radiation and the majority of anticancer agents inflict deleterious DNA damage on tumor cells, predominantly DNA double-strand breaks (DSBs) and covalent. DNA crosslinks. The response of various cancers to genotoxic agents generally reflects cells ability to repair or tolerate DNA damage. Unrepaired persistent DSBs in human cells pose a prominent threat to genomic integrity and cause cell death or senescence. Survival of cancer cells in the face of genotoxic treatment may accelerate tumor progression by forcing clonogenic selection of radio resistant and chemo resistant cells in advanced tumors.

Therefore, regulators of DSB repair processes are highly valuable agents for sensitizing cancerous cells for genotoxic treatment and for treating DSB-associated pathologic disorders.

### SUMMARY OF THE INVENTION

In the first aspect, the invention relates to a method of modulating double stranded DNA breaks (DSB) repair processes in a subject in need thereof. The method of the invention comprises the step of administering to the subject a therapeutically effective amount of at least one compound that modulates the expression or activity of at least one Apolipoprotein B mRNA-editing enzyme-catalytic polypeptide-like (APOBEC) family member, or any composition comprising the same.

In the second aspect, the invention provides an isolated peptide comprising any one of: (a) an amino acid sequence of at least one of Val¹-Lys²-His³-His⁴, as denoted by SEQ ID NO. 66, Lys¹-Gly²-Trp³-Phe⁴ as denoted by SEQ ID NO. 68 and X¹-Leu²-Tyr³-Tyr⁴-Phe⁵ as denoted by SEQ ID NO. 67. In certain embodiments, X₁ may be a positively charged amino acid selected from His and Arg. It should be noted that these peptides were derived from any one of HIV-1 viral infectivity factor (Vif) and APOBEC3F (A3F). In yet other embodiments, the inhibitory peptides of the invention may comprise (b), an amino acid sequence derived from residues 211-240 of A3G, or any fragments, derivatives, homologues, or any combination thereof.

The invention further provides compositions comprising the peptides of the invention as well as combined compositions comprising the modulating compounds of the invention as well as additional therapeutic agents, specifically, genotoxic agents.

In yet another aspect the invention provides a kit modulating DSB repair processes in a subject in need thereof. In certain embodiments, the kit of the invention may comprise (a) at least one compound that modulates the expression or activity of at least one APOBEC family member, and a pharmaceutically acceptable carrier or diluent, optionally, in a first unit dosage form; and (b) at least one therapeutic agent, and a pharmaceutically acceptable carrier or diluent, optionally, in a second unit dosage form.

In another aspect, the invention provides a method of treating a proliferative disorder in a subject in need thereof by inhibiting the cytidine deaminase activity of at least one APOBEC family member, the method comprises the step of: administering to the subject a therapeutically effective amount of any of the inhibiting compounds disclosed herein, specifically, any of the peptides of the invention, or any combination thereof or any composition comprising the same.

Still further, the invention provides methods and uses of a therapeutically effective amount of at least one compound that inhibits the expression or activity of at least one APOBEC family member, in the preparation of a composition for the treatment of a proliferative disorder in a subject being treated with a genotixic therapy.

The invention further provides a method for determining the efficacy of a treatment with a genotixic therapy on a subject suffering from a proliferative disorder comprising the steps of: determining the level of expression of APOBEC3G (A3G) in at least one biological sample of said subject

In yet another aspect the invention provides a method for determining a genotoxic treatment regimen for a subject suffering from a proliferative disorder.

These and other aspects of the invention will become apparent by the hand of the following figures.

### BRIEF DESCRIPTION OF THE FIGURES

FIG. 1A-1D. A3G Multimer Disassembly on ssDNA Termini

FIG. 1A. AFM images showing ssDNA following 1 min incubation with A3G in ice.

FIG. 1B (i and ii). AFM images showing ssDNA following 5 min incubation with A3G in ice. Three-dimensional representation of part of the 2D images are shown (orientation has been tilted for clarity).

FIG. 1C. AFM images showing ssDNA following 30 min incubation with A3G in ice. (i) Inset shows magnification of dashed box area.

FIG. 1D. Quantitation of A3G-ssDNA complexes. For each time point at least 10 different fields were analyzed; Values represent mean±S.D.

Abbreviations: M (A3G multimer); d (dimer); m (monomer).

FIG. 2A-2D. An ssDNA Terminus is Required for Multimeric A3G-ssDNA Interaction

FIG. 2A. AFM images showing M13 linear and circular ssDNA.

FIG. 2B. and FIG. 2C. AFM images showing M13 linear and circular ssDNA following 5 min incubation with A3G.

FIG. 2D. A3G end-dependent association with ssDNA was assessed by electro-mobility shift assay (EMSA). S₁₆₀ oligonucleotide ends (3′, 5′, 3′+5′) were annealed to short complementary oligonucleotides (30 nt) and then incubated with A3G or BSA for 8 min at room temperature (RT). Acrylamide gels were stained with SYBR gold nucleic acid stain.

FIG. 3A-3C. Characterization of A3G W285A Activity and Terminal Cytidine Deamination by A3G

FIG. 3A. Purification of wild-type A3G-His₆ and W285A-His₆ mutant from 293T cells. Shown is Imperial Blue staining of elution fractions resolved by SDS-PAGE. Molecular weights are indicated on the left.

FIG. 3B. Electro-mobility shift assay (EMSA). The indicated proteins were incubated with biotinylated S_{C} oligonucleotides (80 nt) for 8 min at 37° C. and resolved by native PAGE. Acrylamide gels were transferred to nylon membranes and probed by enhanced chemiluminescence.

FIG. 3C. Cytidine deaminase assay scheme is shown (top). The polynucleotide deaminase substrate, comprising an internal cytidine, is cleavage resistant. Upon deamination, the internal uridine-containing polynucleotide is susceptible to cleavage. Activity of purified wild-type and mutant A3G was assessed in a deamination assay (bottom). PAGE analysis of the cleaved deamination products is shown. DNA size is indicated on the left.

Abbreviations: kilo-Dalton (kDa); D (deaminated product); ND (non-deaminated SC substrate); SSB (T4 ssDNA binding protein); by (base-pairs).

FIG. 4A-4D. A3G Association With ssDNA Ends Promotes Terminal Cytidine Deamination and ssDNA Tethering

FIG. 4A. Terminal cytidine deamination by A3G was determined by a DNA polymerase primer extension assay. A primer with 3′-terminal CC was incubated with the indicated proteins and used for PCR amplification of a target sequence. SSB, T4 ssDNA binding protein; DNA size is indicated on the left (bp).

FIG. 4B. Terminal ssDNA tethering was assessed by a plasmid-based end-joining assay. HeLa or HeLa-A3G whole cell extracts were incubated with a pBluescript plasmid linearized with the indicated restriction enzymes. Product sizes of joint linear plasmid (L) are indicated to the right of each PAGE image, as described in the text. UC, uncut plasmid; T4, T4 DNA ligase positive control; DNA marker (M) sizes are indicated (kb).

FIG. 4C. DNA-polymerase extension assay. Schematic depiction of the assay, including expected products migration by PAGE (see text for details). Thirty biotinylated dAMP molecules (shown as circled ‘b’) are incorporated in the DNA1 extension product, and only 21 in the DNA2 extension product.

FIG. 4D. DNA-polymerase extension assay. Denaturing urea-PAGE image showing ssDNA extension products. SsDNA sizes are indicated.

FIG. 5A-5D. Inhibition of A3G Multimer Disassembly and Activation by RPA

FIG. 5A. AFM image showing RPA binding to ssDNA following 30 min incubation in ice.

FIG. 5B. AFM images showing A3G binding to ssDNA following 30 min incubation in ice. A3G was incubated with ssDNA for 1 min followed by 29 min incubation with RPA. Black arrowheads indicate end-bound multimers (A3G or RPA), white arrowheads indicate V-shape protein-ssDNA complexes.

FIG. 5C. Binding of A3G to ssDNA in the presence of RPA was assessed by EMSA. RPA was incubated with S_{c} ssDNA (80 nt) for 30 min (RT), and then mixed with A3G for further 30 min (RT). Monomeric A3G-ssDNA complex is indicated with an arrow. FIG. 5D. Cytidine deamination assay. S_{c} ssDNA was incubated with RPA for 30 min (RT), and then mixed with A3G for further 8 min (37° C.). Cytidine deaminase activity was assessed as the ratio of cleaved deamination product (D, deaminated) to the un-cleaved substrate (ND, not deaminated). Molecular sizes are indicated on the left (bp).

FIG. 6A-6D. A3G Targets Terminal ssDNA During HIV-1 Reverse Transcription

FIG. 6A. Scheme of the endogenous reverse transcription and polymerase extension assays. The viral strong-stop ssDNA (sssDNA) reversed transcribed inside HIV-1 virions on genomic RNA (gRNA) in the presence of encapsidated A3G, serves as an initial primer for polymerase extension of a semi-homologous DNA target.

FIG. 6B. Polymerase extension assay using sssDNA extracted from wild-type (wt) or vif⁽⁻⁾ (ΔV) viruses following endogenous reverse transcription and PCR amplification using the indicated forward primers (pF). PC, positive control oligonucleotide. DNA sizes are indicated on the left (bp).

FIG. 6C. Quantitation of polymerase extension products by real-time PCR. Values were normalized for input ssDNA levels and represent mean±S.D. from three independent experiments performed in duplicates.

FIG. 6D. Exogenous reverse transcription was performed with recombinant HIV-1 reverse transcriptase (RT) using biotinylated (b) oligonucleotides. Oligonucleotide size is indicated (nt).

FIG. 7A-7D. A3G Expression is Inversely Correlated With DSB Occurrence

FIG. 7A. Western blot showing A3G protein level in lymphoma and leukemia cell lines. Alpha-tubulin was used as a loading control. Molecular weights (kDa) are indicated on the left.

FIG. 7B. Indicated cells were exposed to γ-radiation (4 Gy) and stained following 8 h with anti-A3G and anti-γ-H2AX antibodies. Nuclei were counter-stained with DAPI (original magnification ×630).

FIG. 7C. Quantitation of A3G-expression (according to western blot analysis as in FIG. 7A) versus fraction of cells containing DSBs (according to γ-H2AX staining as in FIG. 7B). Values represent mean±S.D. from three independent experiments and at least 10 different fields for each cell line analyzed.

FIG. 7D. Expression level of A3G in lymphoma and leukemia cells correlates with DSB repair. H9 lymphoma cells and CEM-SS and SupT1 leukemia cells were exposed to γ-radiation (4 Gy) and stained following 24 h with anti-A3G and anti-γ-H2AX antibodies. Nuclei were counter-stained with DAPI (original magnification ×630).

FIG. 8A-8J. A3G is Recruited to DSBs and is Required for DSB Repair in Lymphoma Cells

FIG. 8A. Cytoplasmic localization of A3G in non-irradiated blood and H9 cells. Peripheral blood mononuclear cells (PBMCs) were isolated from a healthy donor, activated with phytohemagglutinin (PHA) and stained following 3 days with an anti-A3G antibody. H9 T cells were stained as above and nuclei were counter-stained with DAPI.

FIG. 8B. H9 cells were irradiated (4 Gy) and probed with anti-A3G and anti-γ-H2AX antibodies at the indicated times. Nuclei were counter-stained with DAPI.

FIG. 8C. A3G is recruited to nuclear DNA DSBs. H9 cells were irradiated (4 Gy) or mock-irradiated (No IR) and probed with anti-A3G and anti-γ-H2AX antibodies at the indicated times. Nuclei were counter-stained with DAPI.

FIG. 8D. irradiated cells were harvested at the indicated times after IR and A30 content in nuclear and cytoplasmic fractions was assessed by Western blotting. rt-tubulin and histone H3 were used as cytoplasmic and nuclear markers, respectively. CEM-SS cells not expressing A3D were used as a negative control. Molecular sizes (kDa) are indicated.

FIG. 8E. Scheme of the cytidine deamination assay in which C to U deamination in an oligonucleotide forms a restriction enzyme cleavage site after PCR amplification. ND indicates not dearninated; and D, dearninated (top). Cytidine deaminase activity in extracts of nuclear and cytoplasmic fractions was assessed by incubation with an oligonucleotide substrate, containing a single A30 CCC target site (SEco, 80 nt) for 30 minutes at 37° C. DNA sizes (bp) are indicated on the left. PC indicates positive control oligonucleotide; and NC, negative control oligonucleotide (Bottom).

FIG. 8F. Western blot showing A3G cellular protein level in untreated H9 cells, H9 cells transfected with control siRNA (shCtr1) and H9 cells transfected with A3G-specific siRNA (shA3G), naïve (PBMC(−)) and PHA-activated (PBMC(+)) peripheral blood mononuclear cells, respectively. Alpha-tubulin was used as a loading control. Molecular weights (kDa) are indicated on the left.

FIG. 8G. H9 cells transfected with either shCtr1 or shA3G were irradiated (4 Gy) or mock-irradiated (No IR) and stained with anti-γ-H2AX antibody. Insets are magnifications of dashed box areas showing DAPI counterstaining (top) or γ-H2AX staining (bottom).

FIG. 8H. A3G knockdown or control 1-19 cells were pre-incubated with 20 μM z-VA)-fmk or mock for 1 hour and treated as in FIG. 8G. Values represent mean±SD from 3 independent experiments and at least 10 different fields for each time point analyzed.

FIG. 8I. ARH-77 cells were preincubated with 20 μM z-VAD-fmk or mock for 1 hour and treated as in FIG. 80. Values represent mean±SD from 3 independent experiments and at least 10 different fields for each time point analyzed.

FIG. 8J. Cell cycle analysis following IR. Irradiated (4 Gy) or mock-irradiated (No IR) H9 cells, H9 cells transfected with control siRNA (shCtr1) and H9 cells transfected with A3G-specific siRNA (shA3G), were stained following 20 hours with propidium iodide and DNA content was determined by FACS (10,000 acquired events, left). Values represent mean±S.D. from three independent experiments (right).

FIG. 9A-9B. A3G-Mediated DSB Repair is Cytidine Deaminase Dependent

FIG. 9A. Immunoblot of A3G in SupT11 cells stably expressing an EV control, wild-type A3G, or A3G E259Q catalytic mutant. α-tubulin was used as a loading control.

FIG. 9B. Quantification of γ-H2AX foci in SupT11 cells 24 hours after IR (4 Gy). Values represent mean±SD from 2 independent experiments and at least 10 different fields.

FIG. 10A-10E. Cytidine deamination-dependent recruitment of RPA to ssDNA

FIG. 10A. H9 cells were irradiated (4 Gy) or mock-irradiated and stained following 2 h with anti-A3G and anti-RPA32 antibodies (original magnification ×630). Insets, magnification of areas in the respective irradiated cells.

FIG. 10B. RPA affinity to dU in ssDNA was assessed by EMSA. RPA was incubated with biotinylated S_{C} or S_{U} oligonucleotides (80 nt) at the indicated RPA:DNA molar ratios for 30 min at room temperature. Acrylamide gels were transferred to nylon membranes and probed by enhanced chemiluminescence.

FIG. 10C. AFM image showing RPA (1.2 pmol) binding to L_{C} ssDNA (0.2 pmol). RPA was incubated with the L_{C} DNA for 30 min.

FIG. 10D. AFM images showing RPA (1.2 pmol) and A3G (0.6 pmol) binding to L_{C} ssDNA (0.2 pmol). A3G was incubated with the L_{C} DNA for 30 min followed by incubation with RPA for 30 min in ice.

FIG. 10E. AFM image showing RPA (1.2 pmol) and A3G (0.6 pmol) binding to L_{A} ssDNA (0.2 pmol). A3G was incubated with the L_{A} DNA for 30 min followed by incubation with RPA for 30 min in ice.

Black arrowheads indicate end-bound RPA, white arrowhead indicates V-shape RPA-ssDNA complex; r (internally-bound trimeric RPA); a (monomeric A3G); L_{C} (DNA oligonucleotide comprising CCC); Su (80 nt DNA oligonucleotide containing a single uridine); Sc (80 nt DNA oligonucleotide containing a single cytidine); inset, magnification of dashed box area showing DNA-bound and unbound RPA heterotrimers and A3G monomers.

FIG. 11A-11E. A3G Mediates Deletinal Repair of a Persistant IScel-Induced DSB

FIG. 11A. Scheme of the DR-GFP HR reporter assay. Repair of ISceI-induced DSB by HR reconstitutes the expression of functional GFP.

FIG. 11B. HRind cells were transfected with an EV (empty vector control), A3G (WT), or A3G W285A expression plasmids and induced with TA for 52 hours. GFP expression was measured by PACS. Transfection yield was 55% to 60%, as determined by cotrartsfection with DsRed expression plasmid. Expression of A3G and A3G W285A was evaluated by Western blot (top). Values represent mean±SD from 3 independent experiments. *P=0.007 (unpaired t test).

FIG. 11C. ISceI-expressing lentiviral vector containing long terminal repeats (LTRs), internal ribosomal entry site (1RES), and ISceI target sequence (I) adjacent to GFP.

FIG. 11D. H9 or SupT1 cells were infected with lentiviruses containing the ISceI vector or mock and assessed 48 hours later for GFP⁺ cells by FACS (0 hours). Cells were sorted again 52 hours after induction of ISceI with TA (52 hours). Values represent mean±SD from 3 independent experiments.

FIG. 11E. Genomic DNA was extracted from mock-or ISeI lentivirus-infected H9 or SupT1 52 hours after ISceI induction with TA. *Analysis of PCR amplification of a 5900-bp fragment using vector specific primers was performed by a Bioanalyzer. DNA marker (M) sizes are indicated (kb).

FIG. 12A-12B. A3G Mediates Non-Covalent ssDNA Interstrand Crosslinking

FIG. 12A. AFM images showing association of purified A3G multimers with ssDNA termini (T), or internally bound A3G monomers (m). ICL, interstrand crosslink.

FIG. 12B. A3G W285A catalytic mutant does not mediate ssDNA interstrand crosslinking. AFM images showing association of purified A3G W285A with ssDNA produced by in-vitro rolling circle amplification. A3G W285A multimers are seen as white bulbs. Micrographs are representative of 50 scanned fields.

FIG. 13A-13C. HIV-1 Produced by H9 Cells Incorporates Enzymatically Active A3G Protein

FIG. 13A. Western blots of wild-type or HIV-1Δvif viruses produced by H9 cells. Equal amounts of viral proteins, (20 ng of p24, as measured by p24 antigen capture test) were loaded onto each slot of SDS PAGE. Endogenous A3G and viral p24 CA proteins (upper and lower panels, respectively) were detected by using specific antibodies. Virus harvested from 293T cells transiently co-transfected with HIV-1 Δvif DNA and pcDNA3-A3G-MycHis was used as positive control.

FIG. 13B. Concentrated HIV-1 wt or Δvif virions produced by H9 and SupT1 cells (30 ng of p24) were loaded into the slots of SDS gels. The presence of A3G and Vif proteins in those virus preparations was determined by using polyclonal anti-A3G and anti-Vif antibodies.

FIG. 13C. Deamination of synthetic ss-deoxynucleotide substrate by virus-associated A3G. Equal amounts of HIV-1 wt and Avif viruses (1.25 ng of p24) were added to the reactions containing increasing amounts of the substrate (ranging from 0.01 to 0.2 fmol), as indicated. Abbreviations: PC (positive control); S (an 80 nt-long substrate used for the deamination assay); P (a 40 nt-long product of the restriction reaction).

FIG. 14A-14C. Inhibition of A3G Activity by Vif

FIG. 14A. Recombinant A3G protein (0.75 fmol) was mixed with wt HIV-1 or Δvif virus from SupT1 cells (2.5 ng of p24 per reaction). Reactions were carried out on 1 fmol of A3G oligonucleotides substrate. As negative controls, deamination reactions were loaded with viruses from SupT1 (no A3G). All reactions contained 2.5 ng of p24 (as measured by p24 CA antigen capture test).

FIG. 14B. Purified A3G (0.35 fmol) was incubated with the ss-deoxy-oligonucleotide substrate in the presence of purified Vif for 15 min Lane 1, positive control (dU containing oligonucleotide); Lane 2, negative control (no A3G); Lane 3, sample containing 10 μM BSA; Lane 4, sample containing the elution fraction of Ni—NTA purification from non Vif-expressing bacteria (amount equal to lane 10); Lanes 5-10, dose-dependent inhibition of A3G deamination by increasing Vif concentrations, as indicated.

FIG. 14C. A graphic representation of the Vif-mediated inhibition is shown on the bottom. Values represent the average of triplicates; SD values were less than +/−0.8.

Abbreviations: rec. A3G (Recombinant A3G protein).

FIG. 15A-15C Inhibition of A3G deaminase activity by Vif-derived peptides

FIG. 15A. Fifteen-mer Vif-derived peptides covering the complete Vif sequence were assessed for the inhibition of A3G. The standard deamination reaction was carried out in the presence of 10 μM of each peptide, or an RSV-derived peptide (positive control). PAGE analyses of the cleaved deamination products are shown above the chart.

FIG. 15B. The same reaction as in FIG. 15A was performed in the presence of 1 μM of each peptide, or an RSV-derived peptide (positive control). PAGE analyses of the cleaved deamination products are shown above the chart.

FIG. 15C. the effect of the Vif-derived peptide concentration on A3G mediated deamination. Values represent the average of triplicates; SD values were less than +/−0.5 where not indicated. Abbreviations: P.C (positive control).

FIG. 16A-16B. The Vtf25-39 peptide effectively inhibits cytidine deamination in vitro

FIG. 16A. A standard deamination reaction was carried out in the presence of indicated concentrations of Vif peptide (Vif25-39 or control peptide Vif89-103), with or without A3G. PAGE analysis of the cleaved deamination products is shown. PC (positive control; no Vif peptide); NC (negative control; no A3G).

FIG. 16B. A plot of in vitro A3G deamination activity in the presence of indicated concentrations of Vif peptides.

FIG. 17A-17C. Determination of the Vif and Vif-derived peptides mode of inhibition

FIG. 17A. Deamination of an ss-deoxyoligonucleotide as function of the substrate concentration in the presence of Vif was determined and is shown by double-reciprocal (double-inverse) plot.

FIG. 17B. Deamination of an ss-deoxyoligonucleotide as function of the substrate concentration in the presence of the Vif-derived peptide Vif105-119 was determined and is shown by double-reciprocal (double-inverse) plot.

FIG. 17C. Deamination of an ss-deoxyoligonucleotide as function of the substrate concentration in the presence of the Vif-derived peptide Vif25-39 was determined and is shown by double-reciprocal (double-inverse) plot.

The Vif and Vif-derived peptides concentrations used are indicated. Values represent the average of triplicates; SD values were less than +/−0.4.

FIG. 18A-18C. The Vtf25-39 peptide inhibits DSB repair in vivo

FIG. 18A. H9 cells were incubated for 2 h with the indicated peptides, irradiated (4 Gy) or mock-irradiated (No IR) and stained following 8 h with anti-A3G and anti-γ-H2AX antibodies. Nuclei were counter-stained with DAPI.

FIG. 18B. A plot of the fraction of cells displaying DSBs after treatment with Vif peptides. Values represent mean±S.D. from two independent experiments and at least 10 different fields.

FIG. 18C. Magnification of an irradiated cell pre-incubated with Vif25-39 showing co-localization of A3G and γ-H2AX.

FIG. 19A-19B. Inhibition of A3G deaminase activity by peptides derived from Vif, A3F and A3G

FIG. 19A. Different Vif-derived and A3F-derived peptides were assessed for the inhibition of A3G mediated deamination. The standard deamination reaction was carried out in the presence of 0.001-10 μM of each peptide. Values represent the average of triplicates; SD values were less than +/−0.5 where not indicated.

FIG. 19B. A3G-derived peptides were assessed for the inhibition of A3G mediated deamination. The standard deamination reaction was carried out in the presence of 1-100 μM of each peptide. Values represent the average of triplicates; SD values were less than +/μ0.5 where not indicated.

Abbreviations: P.C (positive control).

### DETAILED DESCRIPTION OF THE INVENTION

DNA double strand breaks (DSBs) may be the most disruptive form of DNA damage. If left unrepaired, they lead to broken chromosomes and cell death. If repaired improperly, they can lead to chromosome translocations and cancer. Humans are at risk for DSBs from exogenous agents. The paradigm agent, ionizing radiation, is present in the environment mainly from the decay of radon gas, which accumulates in homes to different levels depending on the uranium content of the underlying soil. Ionizing radiation is also utilized in medicine for diagnostic x-rays and for treating cancer patients. Anticancer drugs will generate DSBs as well, for example, bleomycin produces oxidative free radicals, which induce strand breaks; etoposide and adriamycin inhibit topoisomerase II to create protein-bridged DSBs. Humans are also at risk for DSBs from endogenous agents. Oxidative metabolism generates free radicals and subsequent strand breaks.

Here the inventors present surprising new data demonstrating a role for APOBEC3G, an antiviral protein, in cellular DSB repair, and provide specific inhibitors of the APOBEC deaminase activity.

Without being bound by any theory, the inventors provide here results which illustrate a general model for A3G activity in DSB repair. IR-induced DNA damage signals import of A3G multimers to the nucleus. A3G multimers, which are catalytically inactive, interact with ssDNA ends at DSB sites and facilitate synapsis of independent ssDNA molecules. Terminal ssDNA binding by multimeric A3G triggers disassembly of the multimer to catalytically active monomers which target internal cytidines in ssDNA. Cytidine deamination of resected ssDNA promotes RPA nucleation which might bind directly to dU or form a base-excision repair complex through interaction with UNG2. RPA then supports monomeric A3G stable DNA end-synapsis, but inhibits disassembly, and hence activation, of incoming A3G multimers. Monomeric A3G tethers two ssDNA termini in a functional synapse, enabling access of DNA polymerase or other repair factors, leading to end-joining RPA, in turn, recruits ATR-ATRIP to ssDNA, which activates checkpoint signaling, and promotes Rad51 presynaptic filament formation and strand exchange, leading to HR. The inventors propose that both end-joining and HR mechanisms are relevant following DSB resection, and may compete in resolving a single DSB.

In view of these results, the modulation of DSB repair in subjects by modulating APOBEC may be plausible. Thus, in the first aspect, the inventors contemplate methods of modulating double stranded DNA break repair processes in a subject in need thereof. The methods comprise the step of administering to the subject a therapeutically effective amount of at least one compound that modulates the expression or activity of at least one Apolipoprotein B mRNA-editing enzyme-catalytic polypeptide-like (APOBEC) family member, or any composition comprising the same.

It should be appreciated that the invention further provides a method for modulating double stranded DNA break repair processes in a mammalian cell. The methods comprise the step of contacting said cells with an effective amount of at least one compound that modulates the expression or activity of at least one APOBEC family member, or any composition comprising the same.

The invention provides methods for modulating DSB repair processes. Three mechanisms exist to repair double-strand breaks (DSBs): non-homologous end joining (NHEJ), microhomology-mediated end joining (MMEJ) and homologous recombination (HR). It should be noted that the present application encompasses modulation of any of these three repair mechanisms.

The methods for modulating DSB repair processes may, according to some embodiments, involve modulating the expression or activity of at least one APOBEC family member. The modulation may be inhibition or alternatively, enhancement of the expression andor the activity of the APOBEC family member, thereby inhibiting or enhancing DSB repair processes in the subject.

It should be appreciated that a compound that either reduces, inhibits, attenuates or alternatively, enhances, augments, increases, induces or elevates the expression or the activity of the APOBEC family member, may be any one of: protein, specifically, peptides, nucleic acid, deoxyribonucleic acid, ribonucleic acid, carbohydrates, lipid, natural organic, synthetically derived organic, inorganic, and peptidomimetics based compounds, a small molecule, a small organic molecule and a non-organic small molecule.

According to some embodiments of the invention, the compound used by the method for modulation of DSB repair, may modulate the activity of at least one APOBEC family member. Such activity may be for example, cytidine deaminase activity. The term “cytidine deaminase activity” as used herein refers to enzymatic activity involving removal of amino group from a cytosine residue creating uridine (C to U).

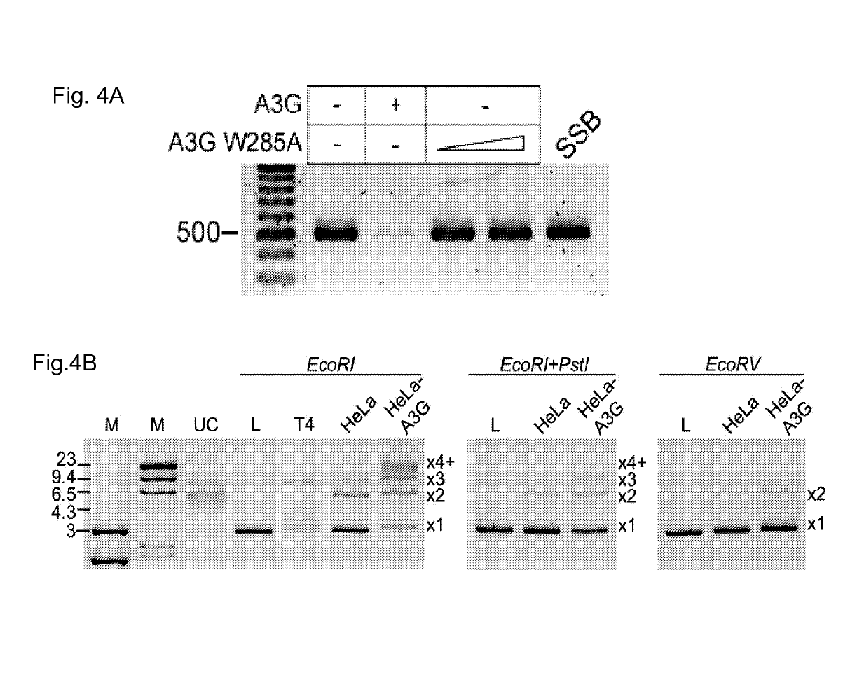

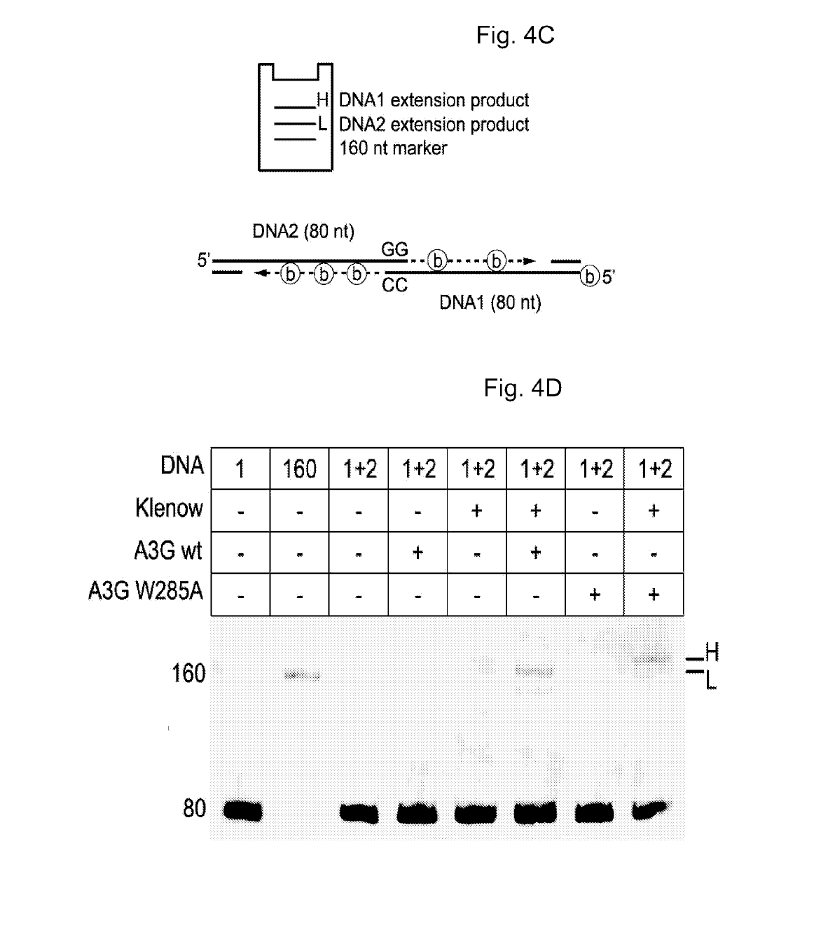

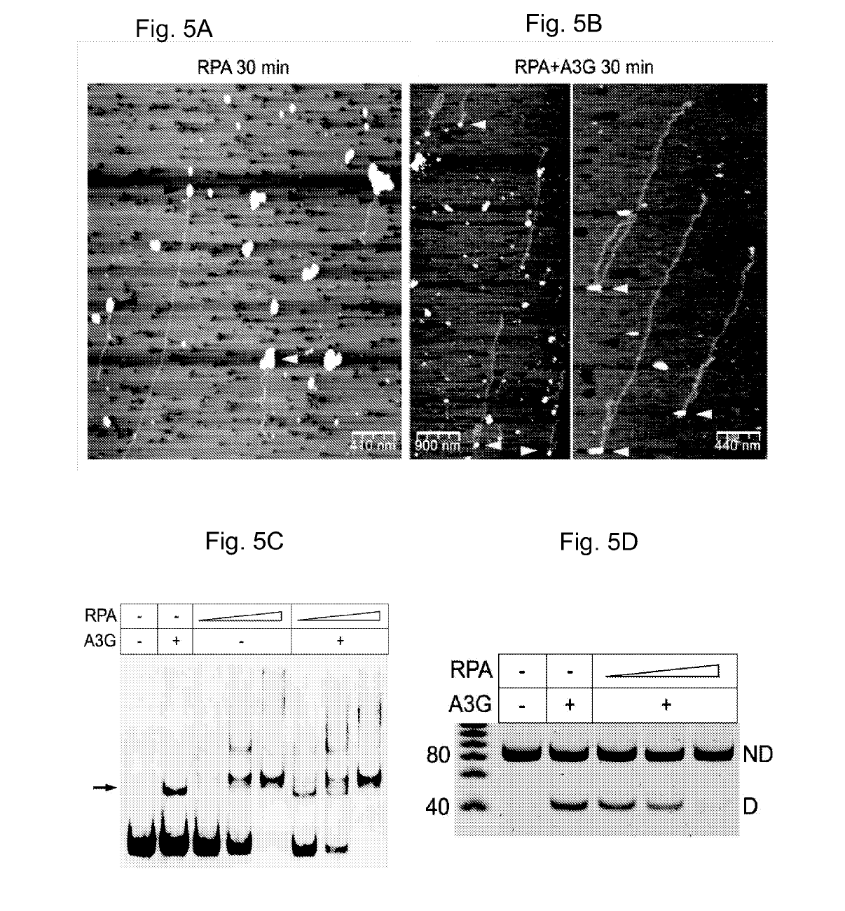

Following A3G multimer disassembly and in the presence of RPA, A3G monomers form stable end-synapses, shown in FIG. 5, in which two ssDNA termini are tethered in close proximity and correct orientation which may direct polymerase extension and functional end-joining, demonstrated in FIG. 4. A3G-mediated formation of random interstrand crosslinks may direct non-templated end joining. The inventors found that A3G mediates DNA polymerase-directed end joining based on minimal terminal homology of only two nucleotides, demonstrated in FIGS. 4C and 4D.

Thus, in other embodiments, the activity modulated by the method of the invention may be single strand DNA (ssDNA) tethering. It should be noted that DNA tethering as used herein is meant bridging remote DNA fragments together in close proximity, thereby enabling end-joining.

The results presented here suggest that A3G may have a dual role in promoting survival of lymphoma cells in-vivo, first by enhancing DSB repair following genotoxic treatment, thus preventing cell death; and secondly by promoting a mutator phenotype, driving tumor progression. Hence, strategies aimed at inhibiting A3G expression or catalytic activity may prove effective in sensitizing lymphoma to genotoxic treatment.

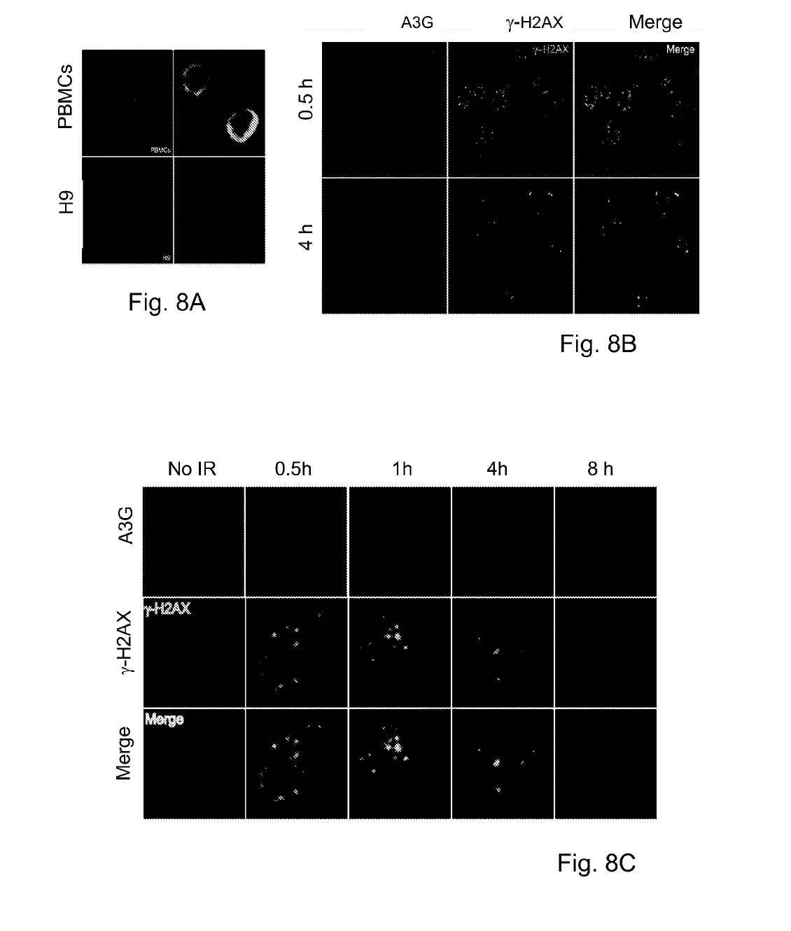

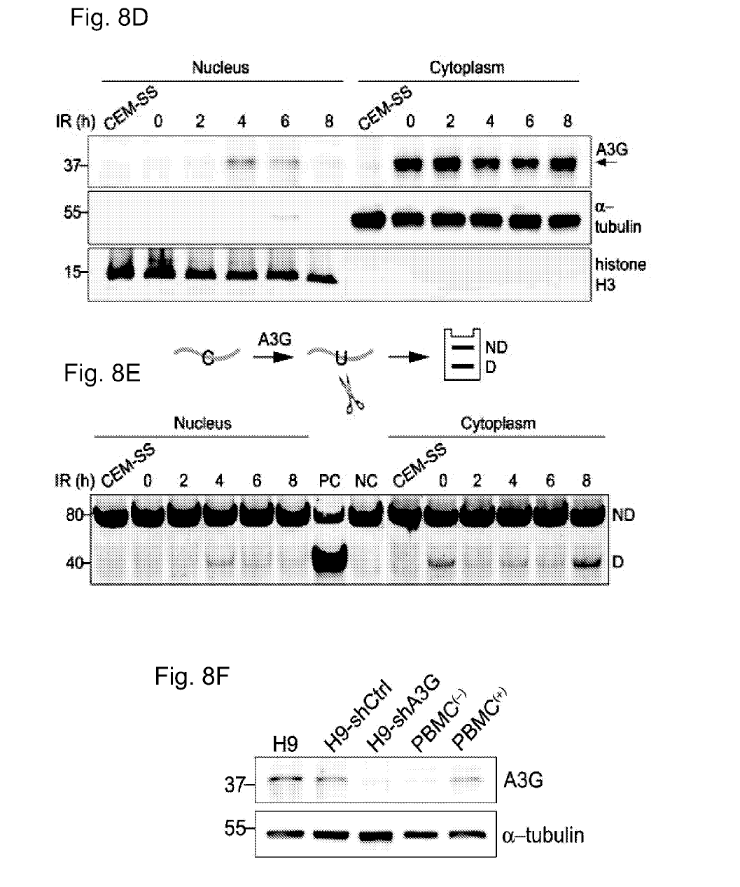

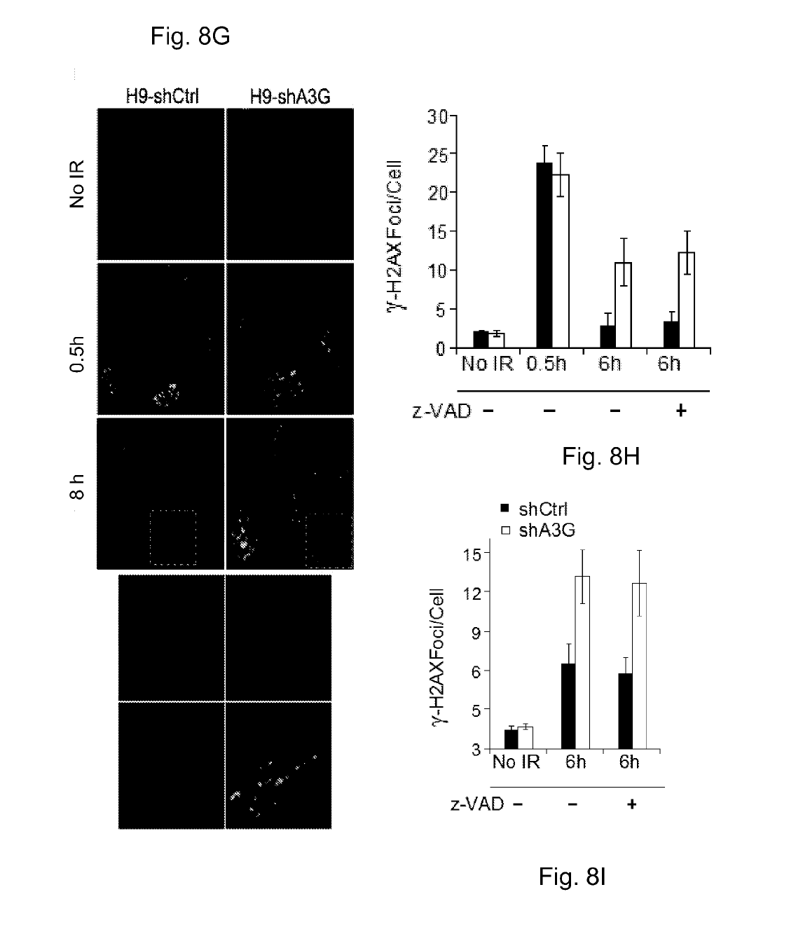

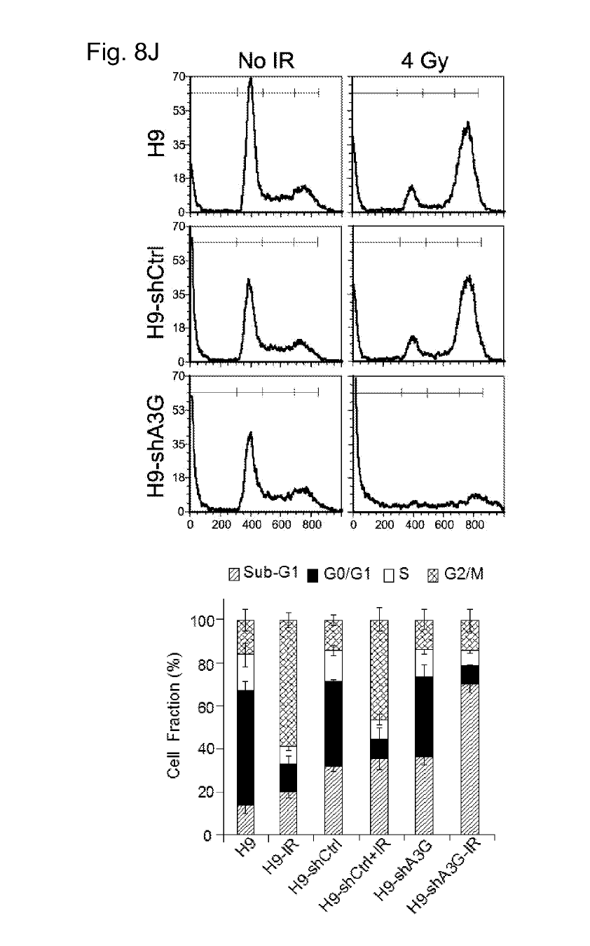

The results presented by the invention show that it is possible to modulate cellular DSB-repair by modulating A3G activity andor expression. Specifically, inhibiting A3G activities (such as, for example, deaminase and DNA tethering activities) or reducing (or blocking) A3G expression level may sensitize cells to DNA damage. More particularly, as shown by FIG. 8J, prevention of A3G expression using siRNA in cells exposed to genotoxic insult inducing stress (for example, ionizing radiation), led to loss of cell cycle control resulting in cell death. This is especially useful for treatment of some cancer types, specifically, cancers which display resistance to chemotherapeutic agents or radiation therapy.

Therefore, according to some embodiments, the invention provides methods for treating, inhibiting, preventing, ameliorating or delaying the onset of a proliferative disorder in a subject in need thereof by inhibiting, reducing or attenuating the DSB repair processes in cells, specifically, malignant cells of the treated subject. In some embodiments, the method comprises the step of administering to the subject a therapeutically effective amount of at least one compound that inhibits, reduces or attenuates the expression or the activity of at least one said APOBEC family member, or any composition comprising the same.

The terms “inhibition”, “moderation” or “attenuation” as referred to herein, relate to the retardation, restraining or reduction of a DSB repair process in the treated subject. Such reduction includes reduction by any one of about 1% to 99.9%, specifically, about 1% to about 95%, about 5% to 90%, about 10% to 85%, about 15% to 80%, about 20% to 75%, about 25% to 70%, about 30% to 65%, about 35% to 60%, about 40% to 55%, about 45% to 50%. More specifically, about 5%, 10%, 15%, 20%, 25%, 30%, 35%, 40%, 45%, 50%, 55%, 60%, 65%, 70%, 75%, 80%, 85%, 90%, 95% or 99.9%.

Inhibition of DSB repair processes by the method of the invention may lead to sensitization of the cancerous cells to a genotoxic insult-inducing treatment which can then be administered to eliminate the sensitized cells. Thus, according to certain embodiments, a combined therapy may be applicable. Such administration of APOBAC inhibitory compound may be performed before, simultaneously with, after or any combination thereof, the genotoxic insult inducing treatment (radiation or chemotherapy). Enhancing sensitization to genotoxic treatment may be particularly applicable for treating proliferative disorders. In certain embodiments, such combined therapy may be specifically voluble for disorders displaying resistance to genotoxic treatment. More specifically, the term “genotoxic treatment” or “genotoxic insult inducing agent” as used herein is defined to include both chemical and physical treatment capable of causing damage to human DNA or the gene. Treatment with carcinogens and mutagens are common examples of chemical genotoxic treatment, while treatment with UV radiation, and X-rays and the like when they produce oxidized DNA product are common examples of physical genotoxic treatment.

Since the goal of the treatment is the destruction of pathologic cells, specifically, cancerous cells sensitized by compounds that disrupt the genome-protective role of APOBEC in DSB repair, according to specific embodiments, the administered genotoxic insult inducing treatment leads to double-stranded-DNA breaks (DSB). Thus, the genotoxic treatment will be at least preferentially, if not selectively toxic towards the sensitized cells. The treatment itself may be at least one of radiation, chemotherapy or any combination thereof.

According to one specific embodiment, the radiation is ionizing radiation, which may be any one of X-rays, gamma rays and charged particles. Ionizing radiation is particularly useful since it induces DSBs effectively, and will therefore damage sensitized cells. In other embodiments, the radiation may be employed in the course of total body irradiation, brachytherapy, radioisotope therapy, external beam radiotherapy, stereotactic radio surgery (SRS), stereotactic body radiation therapy, particle or proton therapy, or body imaging using the ionizing radiation.

An alternative to radiotherapy may be chemotherapeutic treatment. As used herein, a chemotherapeutic agent can be any chemical substance known to be useful for treating cancer, for example, DNA-alkylating agents, anti-tumor antibiotic agents, anti-metabolic agents, tubulin stabilizing agents, tubulin destabilizing agents, hormone antagonist agents, topoisomerase inhibitors, protein kinase inhibitors, HMG-CoA inhibitors, (IDK inhibitors, cyclin inhibitors, caspase inhibitors, metalloproteinase inhibitors, antisense nucleic acids, triple-helix DNAs, nucleic acids aptarners, and molecularly-modified viral, bacterial or exotoxic agents. Examples of particularly suitable agents for use in the methods of the present invention include, but are not limited to, at least one of cisplatin, carboplatin, oxaliplatin, mechlorethamine, cyclophosphamide, chlorambucil, ifosfamide, teniposide, irinotecan, amsacrine, etoposide phosphate, topotecan, dactinomycin, doxorubicin, epirubicin and bleomycin, as considered by some embodiments.

According to one embodiment, the proliferative disorder treated by the method of the invention may be any one of lymphoma, carcinoma, sarcoma, melanoma, leukemia and myeloma.

As used herein to describe the present invention, “proliferative disorder”, “cancer”, “tumor” and “malignancy” all relate equivalently to a hyperplasia of a tissue or organ. If the tissue is a part of the lymphatic or immune systems, malignant cells may include non-solid tumors of circulating cells. Malignancies of other tissues or organs may produce solid tumors. In general, the compositions and methods of the present invention may be used in the treatment of non-solid and solid tumors.

Malignancy, as contemplated in the present invention may be any one of lymphomas, leukemias, carcinomas, melanomas, myeloma and sarcomas.

Lymphoma is a cancer in the lymphatic cells of the immune system. Typically, lymphomas present as a solid tumor of lymphoid cells. These malignant cells often originate in lymph nodes, presenting as an enlargement of the node (a tumor). It can also affect other organs in which case it is referred to as extranodal lymphoma. Non limiting examples for lymphoma include Hodgkin's disease, non-Hodgkin's lymphomas and Burkitt's lymphoma.

Carcinoma as used herein, refers to an invasive malignant tumor consisting of transformed epithelial cells. Alternatively, it refers to a malignant tumor composed of transformed cells of unknown histogenesis, but which possess specific molecular or histological characteristics that are associated with epithelial cells, such as the production of cytokeratins or intercellular bridges.

Melanoma as used herein is a malignant tumor of melanocytes. Melanocytes are cells that produce the dark pigment, melanin, which is responsible for the color of skin. They predominantly occur in skin, but are also found in other parts of the body, including the bowel and the eye. Melanoma can occur in any part of the body that contains melanocytes.

Leukemia refers to progressive, malignant diseases of the blood-forming organs and is generally characterized by a distorted proliferation and development of leukocytes and their precursors in the blood and bone marrow. Leukemia is generally clinically classified on the basis of (1) the duration and character of the disease-acute or chronic; (2) the type of cell involved; myeloid (myelogenous), lymphoid (lymphogenous), or monocytic; and (3) the increase or non-increase in the number of abnormal cells in the blood-leukemic or aleukemic (subleukemic).

Sarcoma is a cancer that arises from transformed connective tissue cells. These cells originate from embryonic mesoderm, or middle layer, which forms the bone, cartilage, and fat tissues. This is in contrast to carcinomas, which originate in the epithelium. The epithelium lines the surface of structures throughout the body, and is the origin of cancers in the breast, colon, and pancreas.

Myeloma as mentioned herein is a cancer of plasma cells, a type of white blood cell normally responsible for the production of antibodies. Collections of abnormal cells accumulate in bones, where they cause bone lesions, and in the bone marrow where they interfere with the production of normal blood cells. Most cases of myeloma also feature the production of a paraprotein, an abnormal antibody that can cause kidney problems and interferes with the production of normal antibodies leading to immunodeficiency. Hypercalcemia (high calcium levels) is often encountered.

Further malignancies that may find utility in the present invention can comprise but are not limited to hematological malignancies (including lymphoma, leukemia and myeloproliferative disorders), hypoplastic and aplastic anemia (both virally induced and idiopathic), myelodysplastic syndromes, all types of paraneoplastic syndromes (both immune mediated and idiopathic) and solid tumors (including GI tract, colon, lung, liver, breast, prostate, pancreas and Kaposi's sarcoma. More particularly, the malignant disorder may be lymphoma. Non-limiting examples of cancers treatable according to the invention include hematopoietic malignancies such as all types of lymphomas, leukemia, e.g. acute lymphocytic leukemia (ALL), acute myelogenous leukemia (AML), chronic lymphocytic leukemia (CLL), chronic myelogenous leukemia (CML), myelodysplastic syndrome (MDS), mast cell leukemia, hairy cell leukemia, Hodgkin's disease, non-Hodgkin's lymphomas, Burkitt's lymphoma and multiple myeloma, as well as for the treatment or inhibition of solid tumors such as tumors in lip and oral cavity, pharynx, larynx, paranasal sinuses, major salivary glands, thyroid gland, esophagus, stomach, small intestine, colon, colorectum, anal canal, liver, gallbladder, extraliepatic bile ducts, ampulla of vater, exocrine pancreas, lung, pleural mesothelioma, bone, soft tissue sarcoma, carcinoma and malignant melanoma of the skin, breast, vulva, vagina, cervix uteri, corpus uteri, ovary, fallopian tube, gestational trophoblastic tumors, penis, prostate, testis, kidney, renal pelvis, ureter, urinary bladder, urethra, carcinoma of the eyelid, carcinoma of the conjunctiva, malignant melanoma of the conjunctiva, malignant melanoma of the uvea, retinoblastoma, carcinoma of the lacrimal gland, sarcoma of the orbit, brain, spinal cord, vascular system, hemangiosarcoma and Kaposi's sarcoma.

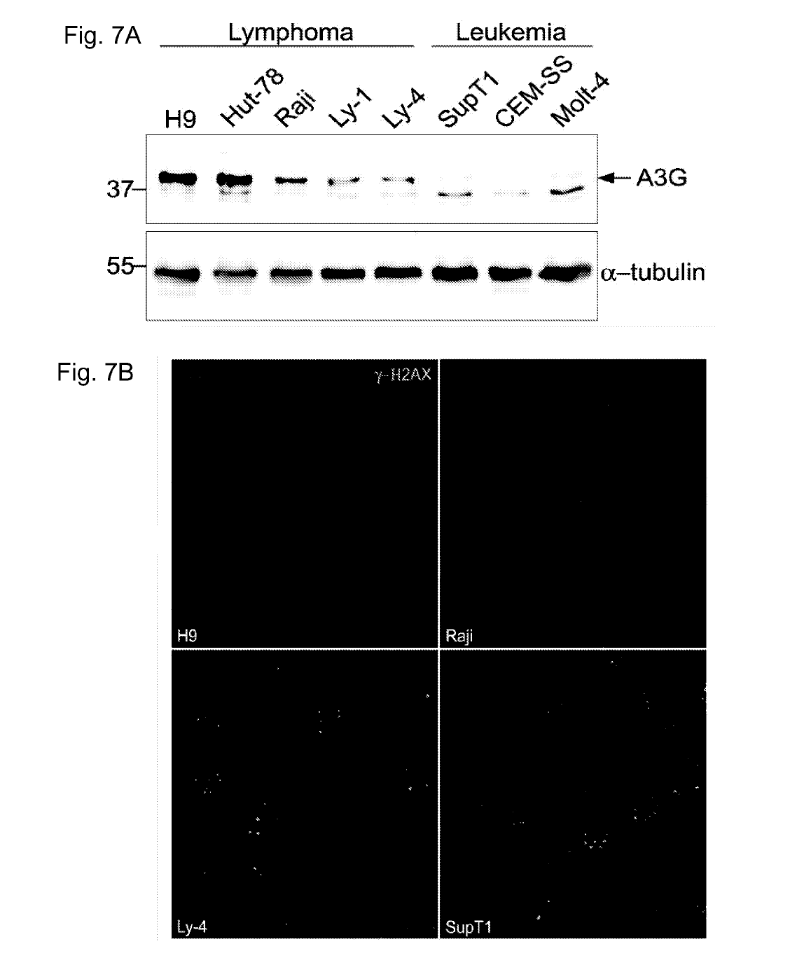

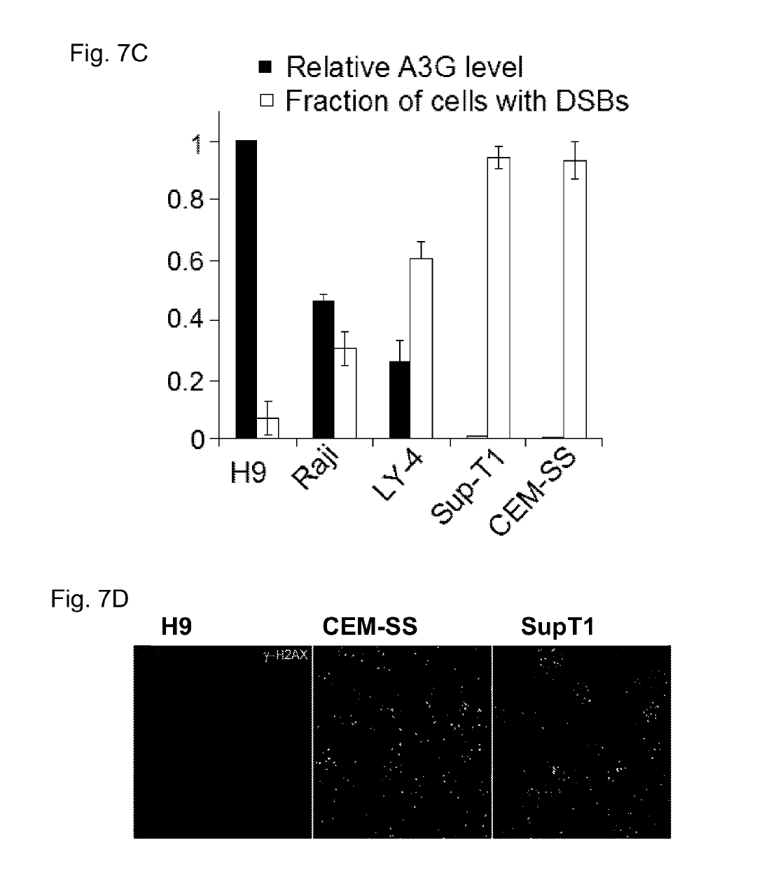

As shown in FIG. 7B, lymphoma cells appear to express higher levels of A3G and display lower DSB frequency than leukemia cells which express low levels of A3G and display high DSB frequency. It is worthwhile mentioning that lymphoma display resistance to chemotherapeutic agents. Thus, inhibiting A3G activity or expression may sensitize tumors which express high A3G levels to chemotherapeutic agents which damage DNA. Therefore, in one specific embodiment, the method of the invention may be particularly efficient in the treatment of proliferative disorders such as lymphoma, by sensitizing pathogenic, specifically, malignant cells to genotoxic insults inducing treatment.

Lymphoma is a cancer in the lymphatic of the immune system and presents as a solid tumor of lymphoid cells. It is treatable with chemotherapy, and in some cases radiotherapy andor bone marrow transplantation, and can be curable depending on the histology, type, and stage of the disease. These malignant cells often originate in lymph nodes, presenting as an enlargement of the node (a tumor). Lymphomas are closely related to lymphoid leukemias, which also originate in lymphocytes but typically involve only circulating blood and the bone marrow (where blood cells are generated in a process termed haematopoesis) and do not usually form static tumors. There are many types of lymphomas, and in turn, lymphomas are a part of the broad group of diseases called hematological neoplasms.

Hodgkin's lymphoma, previously known as Hodgkin's disease, is a type of lymphoma, which is a cancer originating from white blood cells called lymphocytes. Hodgkin's lymphoma is characterized by the orderly spread of disease from one lymph node group to another and by the development of systemic symptoms with advanced disease. When Hodgkin's cells are examined microscopically, multinucleated Reed-Sternberg cells (RS cells) are the characteristic histopathologic finding. Hodgkin's lymphoma may be treated with radiation therapy, chemotherapy or hematopoietic stem cell transplantation, the choice of treatment depending on the age and sex of the patient and the stage, bulk and histological subtype of the disease. Therefore, according to certain embodiments, the method of the invention may be particularly applicable in the treatment of Hodgkin's lymphoma.

The non-Hodgkin lymphomas (NHLs) are a diverse group of blood cancers that include any kind of lymphoma except Hodgkin's lymphomas. Types of NHL vary significantly in their severity, from indolent to very aggressive. According to certain embodiments, the method of the invention may be particularly applicable in the treatment of NHL.

Lymphomas are treated by combinations of chemotherapy, monoclonal antibodies, immunotherapy, radiation, and hematopoietic stem cell transplantation.

In yet another embodiment, the method of the invention may be applicable for treating genotoxic-drug resistant proliferative disorders. It should be noted that the term “drug resistant” as used herein refers to the ability of cells (or a patient) to resist or to overcome the effect of a specific drug. More specifically, “genotoxic-drug resistance” reflects enhanced ability to resist a drug that cause damage to DNA, for example by enhancing DSB repair processes.

Some embodiments of the invention contemplate a treatment of a subject suffering from a proliferative disorder, specifically, lymphoma, with inhibitors of an AOPOBAC family member, specifically, A3G activity according to the invention, wherein the treatment results in sensitization of the malignant cells to treatment with genotoxic insult inducing agent, and thereby, the inhibition of abnormal cellular proliferation by about 1% to 99.9%, specifically, about 1% to about 95%, about 5% to 90%, about 10% to 85%, about 15% to 80%, about 20% to 75%, about 25% to 70%, about 30% to 65%, about 35% to 60%, about 40% to 55%, about 45% to 50%. More specifically, about 5%, 10%, 15%, 20%, 25%, 30%, 35%, 40%, 45%, 50%, 55%, 60%, 65%, 70%, 75%, 80%, 85%, 90%, 95% or 99.9%.

With regards to the above, it is to be understood that, where provided, percentage values such as, for example, 10%, 50%, 120%, 500%, etc., are interchangeable with “fold change” values, i.e., 0.1, 0.5, 1.2, 5, etc., respectively.

As noted above, the method of modulating double stranded DNA break (DSB) repair processes in a subject in need thereof comprises administering to the subject a therapeutically effective amount of at least one compound that modulates the expression or activity of at least one Apolipoprotein B mRNA-editing enzyme-catalytic polypeptide-like (APOBEC) family member. In certain embodiments, the APOBEC family member referred to herein, may be a member of the APOBEC3 (A3) family. As noted above, this family comprises seven APOBEC3 (A3) members, designated A3A, B, C, D, E, F, G and H, as denoted by GenBank Accession Nos. P31941, Q9UH17, Q9NRW3, Q96AK3, Q8IUX4, Q9HC16, Q6NTF7, respectively. It should be appreciated that in certain embodiments, as used herein in the specification and in the claim section below, APOBAC3 protein refers to the human A3G (also denoted by SEQ ID NO. 88). More specifically, the human A3G protein comprises an amino acid sequence of 384 amino acid residues as denoted by GenBank Accession No. Q9HC16, encoded by a nucleic acid sequence of 1848 by linear mRNA, as denoted by Accession: NM_{—}021822. It should be further appreciated that the Homo sapiens A3G, Gene ID as indicated by the NCBI Gene database, is 60489.

In Examples 12 and 13, the inventors demonstrate that Vif inhibits the APOBEC deaminase activity, which as shown by the invention is important for DSB repair by recruiting RPA. Accordingly, particular embodiments envision the method of modulating double stranded DNA break (DSB), wherein the compound that inhibits the cytidine deaminase activity of the APOBEC family member may be a retrovirus viral infectivity factor (Vif), lentivirus Vif, and specifically, HIV-1 HXBII Vif polypeptide, or any functional fragment, peptide, derivative or homologue thereof, or any combination thereof. According to one specific embodiment, the HIV-1 Vif is denoted by Uniprot accession no. P69723, also as denoted by NP_{—}057851 and by SEQ ID NO. 50.

In yet another embodiment, such inhibitor may be a peptide derived from an APOBAC family member.

Example 14 also provides information suggesting that specific peptides comprised in Vif are capable of inhibiting APOBEC cytidine deaminase activity. Thus, the invention further provides method for modulating DSB repair in a subject in need thereof by sensitizing cells to genotoxic treatment using specific HIV Vif peptides. In certain embodiments such Vif derived peptides may comprise the amino acid sequence of any one of residues 25-39, 105-119, 107-115, 9-23, 37-51, 101-115, 118-127, 1-15, 5-19, 13-27, 17-31, 21-35, 30-39, 33-39, 36-39, 23-30, 25-28, 25-34, 29-43, 33-47, 41-55, 45-59, 49-63, 53-67, 57-71, 61-75, 65-79, 69-83, 73-87, 77-91, 81-95, 85-99, 89-103, 93-107, 97-111, 105-119, 108-113, 108-112, 109-115, 109-117, 109-112, 109-123, 117-131, 121-135, 125-139, 129-143, 133-147, 137-151, 141-155, 145-159, 149-163, 153-167, 157-171, 161-175, 165-179, 169-183, 173-187, 177-191, 181-192, of HIV-1 Vif, residues 304-312, 305-311, 224-231, of APOBEC3F (A3F) and residues 226-240, 211-225 or 226-231, of A3G, any derivatives and fragments thereof or any combination thereof.

This Example also demonstrates that particular Vif peptides are efficient in inhibiting APOBEC cytidine deaminase activity. Accordingly, in some embodiments, the method for modulating DSB repair in a subject in need thereof, may comprise the use of a peptide comprising the amino acid sequence of any one of residues 25-39, 105-119, 9-23, 37-51, 101-115, 118-127, 1-15, 5-19, 13-27, 17-31, 21-35, 29-43, 33-47, 41-55, 45-59, 49-63, 117-131, 121-135 and 125-139 of HIV-1 Vif, or any derivatives, homologues, or any combination thereof.

Furthermore, in Example 14, the inventors show the specific inhibition of APOBEC by various Vif fragments and peptides. For instance, it is shown that Vif fragments comprising sequences derived from residues 1-51 andor 101-127 of HIV-1 Vif, inhibit APOBEC deaminase activity. Thus, in some embodiments, the method for modulating DSB repair contemplated by the invention comprises the use of Vif functional fragments comprised within the amino acid sequence of any one of residues 1-51 and 101-127 of HIV-1 Vif, as denoted by SEQ ID NOs. 51 and 52, respectively.

More specifically, six peptides inhibited the A3G deaminase activity at a lower concentration of 1 μM, mapping the inhibitory sequences to Vif9-23 (SEQ ID NO.:3), Vif25-39 (SEQ ID NO.:7) and Vif37-51 (SEQ ID NO.:10) at the N-terminal region, and Vif101-115 (SEQ ID NO.:26), Vif105-119 (SEQ ID NO.:27 and Vif113-127 (SEQ ID NO.:29) at the central region.

As shown in Example 17, the inventors further demonstrated inhibition of A3G cytidine deaminase activity using different shorter peptides derived from Vif, as well as from A3F and A3G, thereby establishing a consensus sequence for these peptides. Thus, according to some embodiments, the inhibitory peptides used by the method of the invention may comprise any one of: (a) an amino acid sequence of at least one of Val^{e}Lys²His³His⁴, as denoted by SEQ ID NO. 66, Lys¹-Gly²-Trp³-Phe⁴ as denoted by SEQ ID NO. 68 and X¹-Leu²-Tyr³-Tyr⁴-Phe⁵ as denoted by SEQ ID NO. 67, wherein X₁ is a positively charged amino acid selected from His and Arg; and wherein said peptide is derived from any one of HIV-1 viral infectivity factor (Vif) and APOBEC3F (A3F); and (b) a peptide derived from residues 211-240 of A3G.

In particular embodiments, the method for modulating DSB repair and thereby sensitizing pathologic cells of the treated subject to genotoxic treatment, involves the use of peptides comprising the amino acid sequence of any one of residues 25-39, 105-119 and 107-115 of HIV-1 Vif, residues 304-312, 305-311 and 224-231 of A3F, residues 226-240, 211-225 and 226-231 of A3G, or any fragments, derivatives, homologues, or any combination thereof.

In yet other specific embodiments, a peptide used by the method of the invention may comprise an amino acid sequence of any one of residues 25-39, 105-119 and 107-115 of HIV-1 Vif, as denoted by SEQ ID NO. 7, 27 and 71, respectively, residues 304-312 and 305-311 of A3F as denoted by SEQ ID NO. 74 and 75, respectively and residues 211-225 and 226-240 of A3G as denoted by SEQ ID NO. 83 and 84, respectively, or any fragments, derivatives, homologues, or any combination thereof.

In one specific embodiment the invention provides methods using a Vif derived peptide comprising the amino acid sequence of residues 25-39 of HIV-1 Vif that has the amino acid sequence of Val-Lys-His-His-Met-Tyr-Ile-Ser-Gly-Lys-Ala-Lys-Gly-Trp-Phe as denoted by SEQ ID NO.:7, or any derivatives, homologues, or any combination thereof.

In yet another specific embodiment, the method of the invention involves the use of a peptide comprising the amino acid sequence of residues 105-119 of HIV-1 Vif that has the amino acid sequence of Gln-Leu-Ile-His-Leu-Tyr-Tyr-Phe-Asp-Cys-Phe-Ser-Glu-Ser-Ala as denoted by SEQ ID NO.:27, or any fragments, derivatives, homologues, or any combination thereof.

In some specific embodiments, the peptide used by the method of the invention is a peptide derived from residues 107-115 of HIV-1 Vif having the amino acid sequence of Ile-His-Leu-Tyr-Tyr-Phe-Asp-Cys-Phe as denoted by SEQ ID. NO. 71.

In another embodiment, the peptide used by the method of the invention is derived from residues 304-312 of A3F, and has the amino acid sequence of Ala-Arg-Leu-Tyr-Tyr-Phe-Trp-Asp-Thr as denoted by SEQ ID. NO. 74.

In a further embodiment, the peptide used by the method of the invention is derived from residues 305-311 of A3F and has the amino acid sequence of Arg-Leu-Tyr-Tyr-Phe-Trp-Asp as denoted by SEQ ID. NO. 75.

In a further embodiment, the peptide used by the method of the invention is derived from residues 211-225 of A3G and has the amino acid sequence of Trp-Val-Arg-Gly-Arg-His-Glu-Thr-Tyr-Leu-Cys-Tyr-Glu-Val-Glu as denoted by SEQ ID. NO. 83.

In a further embodiment, the peptide used by the method of the invention is derived from residues 226-240 of A3G and has the amino acid sequence of Arg-Met-His-Asn-Asp-Thr-Trp-Val-Leu-Leu-Asn-Gln-Arg-Arg-Gly as denoted by SEQ ID. NO. 84.

It should be appreciated that the method of the invention may also use any combination of any of the Vif-derived peptides described herein.

In certain embodiments, the peptides used by the methods of the invention and the compositions described herein after, may be isolated and purified peptides.

Certain embodiments of the invention involve the use of peptides for the methods and as will be described herein after, also the compositions of the invention. It should be appreciated that such peptides or amino acid sequences are preferably isolated and purified molecules, as defined herein. The term “purified” or “isolated” refers to molecules, such as amino acid sequences, or peptides that are removed from their natural environment, isolated or separated. An “isolated peptide” is therefore a purified amino acid sequence. “Substantially purified” molecules are at least 60% free, preferably at least 75% free, and more preferably at least 90% free from other components with which they are naturally associated. As used herein, the term “purified” or “to purify” also refers to the removal of contaminants from a sample.

As shown in Example 9 and FIGS. 8F-8G, introduction of a specific shRNA molecule inhibiting the expression of A3G, to cells, inhibited the expression of A3G leading to inhibition of DSB repair mediated by A3G. Therefore, in other embodiments, the method for modulating DSB repair provided by the invention may comprise the use of a compound that inhibits, reduces or attenuates the expression of at least one said APOBEC family member. In some embodiments, the inhibitor used by the invention may be an agent that down regulates the expression of A3G by RNA silencing. As used herein, the phrase “RNA silencing” refers to a group of regulatory mechanisms [e.g. RNA interference (RNAi), transcriptional gene silencing (TGS), post-transcriptional gene silencing (PTGS), quelling, co-suppression, and translational repression] mediated by RNA molecules which result in the inhibition or “silencing” of the expression of a corresponding target gene, specifically, the genes encoding A3G. In certain embodiments, the RNA silencing agent is capable of preventing complete processing (e.g., the full translation andor expression) of an mRNA molecule through a post-transcriptional silencing mechanism. Thus, in certain embodiments, the inhibitory compound may be at least one nucleic acid-APOBEC-specific inhibitor selected from shRNA, siRNA, ribozyme or antisense RNA, or any functional fragments thereof, or any combination thereof, or any vector comprising the same. More specifically, the compound may be APOBEC3G-specific shRNA or any vector comprising the same. In some specific embodiment such shRNA molecule may be an A3G-specific pLKO.1.shA3G (TRCN0000052188, clone NM_{—}021822.1-398s1c1), as denoted by SEQ ID NO. 92.

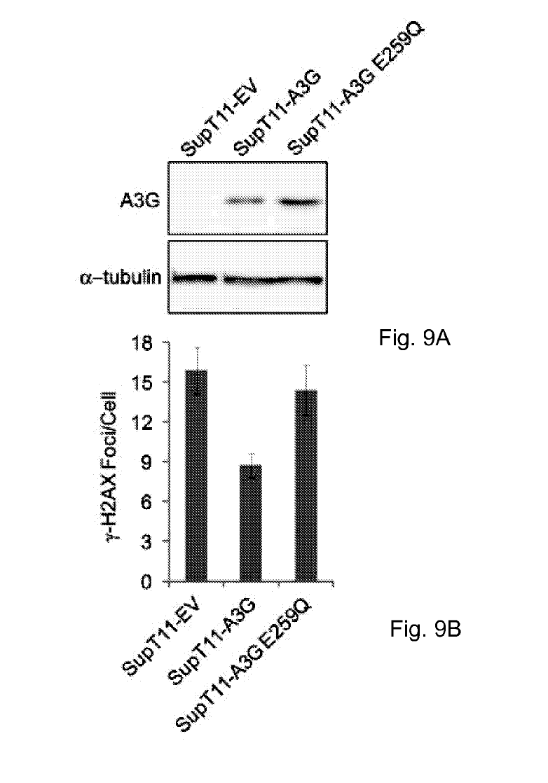

Example 3 shows that the A3G mutated molecule comprising the W285A substitution, retains the ability to bind ssDNA but is devoid of cytidine deaminase activity, and therefore fails to mediate DSB repair process. Moreover, FIG. 9 demonstrates the use of another A3G mutant, E259Q, having an impaired ability to mediate DSB repair. Therefore, mutated A3G molecules that retain the ability to bind ssDNA may compete with the wild type A3G molecule in the cell and inhibit DSB repair mediated thereby. Thus, according to certain embodiments, the method of the invention may use a mutated A3G molecule devoid of cytidine deaminase activity. More specific embodiments relate to A3G mutant comprising at least one of W285A and E259Q point mutations or substitutions. More specific embodiments relate to mutants comprising the amino acid sequence of any one of SEQ ID NO. 81 and 90.

Treatment with chemotherapy alone is limited in that cancer cells often become resistant to a broad spectrum of structurally unrelated chemotherapeutic agents. Such resistance, termed “multidrug resistance” (MDR), is a common problem in the treatment of patients with cancer, and the resistance of tumor cells to chemotherapeutic drugs represents a major problem in clinical oncology. Thus, in yet another aspect, the invention further provides a method for sensitization of a subject suffering from a proliferative disorder to a genotoxic treatment. The method comprises the step of administering to said subject a therapeutically effective amount of at least one compound that inhibits the expression or the activity of at least one said APOBEC family member, or of any composition comprising the same. It should be noted that any inhibitory compound described by the invention may be applicable for such method. In certain embodiments, the compound used by the method of the invention may be administered before, simultaneously with, after or any combination thereof, with said genotoxic treatment. As used herein “sensitization” refers to enhancement of the effect of a specific drug on cells. Increased sensitivity may result in reducing the amount of a genotoxic agent required for achieving the desired therapeutic effect.

It should be noted that in certain embodiments, the method is applicable for subjects suffering from a genotoxic-drug resistant proliferative disorder.

The present invention demonstrates for the first time, the pivotal role of APOBEC3 in DSB repair processes and thereby provides methods for modulating DSB repair processes in a subject in need thereof. More specifically, modulating APOBEC expression, deaminase activity and DNA tethering may be beneficial for treatment of a wide variety of pathological conditions stemming from DSBs, or uncontrolled DSB processes. Indeed, anomalies in DNA double strand breaks repair can lead to several human diseases such as Ataxia telangiectasia, Nijmegen breakage syndrome, Fanconi anemia and chromosomal translocations in general, which, in turn, may lead to cancer and SCID. DSBs may be also associated with aging and cell senescence.

More specifically, for treating disorders associated with DSB damage, enhancement of DSB repair processes may be desired. Thus, in one embodiment, the invention provides a method for treating, inhibiting, preventing, ameliorating or delaying the onset of a disorder associated with DSB damage by administering to a subject suffering of such disorder, a compound that modulates the expression or deaminase activity of at least one said APOBEC family member, or any composition comprising the same. In one specific embodiment, such modulation may be an increase, augmentation or induction of the expression or activity of at least one said APOBEC family member in the subject in need thereof.

In one particular embodiment, such increase may involve administration of a therapeutic effective amount of APOBAC3 or of any combination thereof with at least one agent involved in DNA repair processes, for example, at least one of RPA, ATM, DNA-PK, MRN complex, Rad51, Rad52 and also ATR. It should be appreciated that the invention contemplates the use of APOBEC3 protein or any functional fragment or derivative thereof or any construct encoding the APOBEC3, IA3G, specifically, as denoted by SEQ ID NO. 88. In one specific embodiment, the invention contemplates the use of A3G with RPA in methods inducing a DSB repair process. In more specific embodiments, the RPA molecule referred to herein is the human RPA molecule. More specifically, the human RPA protein as denoted by GenBank Accession No. NM_{—}002945.3 and SEQ ID NO 91.

It should be appreciated that the administration of a therapeutically effective amount of APOBAC3 or any combination thereof with at least one agent involved in DNA repair processes may be particularly useful for treatment, inhibition, amelioration, prophylaxis or delaying the onset of ionizing radiation-induced DNA damage and associated conditions. For example, administration of A3G or any fragment thereof or any combination thereof with an additional therapeutic agent, or of any composition comprising the same, may be useful as a protective or therapeutic measure for workers handling radioactive material, or for military personnel and civilian population at risk of nuclear attack.

It should be noted that any compound that enhance, increase or elevates the activity andor expression of A3G, is also applicable in treating conditions associated with ionizing radiation-induced DNA damage, as well as in DSB-repair associated disorders, as described by the invention.

The invention provides methods for treating DSB-repair associated disorders. It is understood that the interchangeably used terms “associated” and “related”, when referring to pathologies herein, mean diseases, disorders, conditions, or any pathologies which at least one of: share causalities, co-exist at a higher than coincidental frequency, or where at least one disease, disorder condition or pathology causes the second disease, disorder, condition or pathology.

The methods of the invention involve administration of therapeutically effective amount of A3G modulating compounds of the invention (for example, the Vif, A3F and A3G peptides, the APOBEC specific siRNA that inhibit the activity and expression or the mutated A3G molecules, respectively, of APOBEC, or alternatively, any compound that increases activation or expression of APOBEC) or any compositions thereof. The term “effective amount” as used herein is that determined by such considerations as are known to the man of skill in the art. The amount must be sufficient to prevent or ameliorate tissue damage caused by proliferative disorders and DSB-related disorders treated, for example, lymphoma and Ataxia telangiectasia, for example. Dosing is dependent on the severity of the symptoms and on the responsiveness of the subject to the active drug. Medically trained professionals can easily determine the optimum dosage, dosing methodology and repetition rates. In any case, the attending physician, taking into consideration the age, sex, weight and state of the disease of the subject to be treated, will determine the dose. Optimal dosing schedules can be calculated from measurements of drug accumulation in the body of the patient. Persons of ordinary skill can easily determine optimum dosages, dosing methodologies and repetition rates. In general, dosage is calculated according to body weight, and may be given once or more daily, weekly, monthly or yearly, or even once every 2 to 20 years. Persons of ordinary skill in the art can easily estimate repetition rates for dosing based on measured residence times and concentrations of the combined composition of the invention in bodily fluids or tissues. Following successful treatment, it may be desirable to have the patient undergo maintenance therapy to prevent the recurrence of the disease state, wherein the A3G modulating compound used by the method of the invention is administered in maintenance doses, once or more daily. As use herein “therapeutically effective amount” means an amount of the peptide or a composition comprising such peptide which provides a medical benefit as noted by the clinician or other qualified observer. Regression of a tumor in a patient is typically measured with reference to the diameter of a tumor. Decrease in the diameter of a tumor indicates regression. Complete regression is also indicated by failure of tumors to reoccur after treatment has stopped.

The present invention provides methods for treating proliferative disorders. The term “treatment or prevention” refers to the complete range of therapeutically positive effects of administrating to a subject including inhibition, reduction of, alleviation of, and relief from, proliferative disorder symptoms or undesired side effects of proliferative disorder related disorders. More specifically, treatment or prevention includes the prevention or postponement of development of the disease, prevention or postponement of development of symptoms andor a reduction in the severity of such symptoms that will or are expected to develop. These further include ameliorating existing symptoms, preventing-additional symptoms and ameliorating or preventing the underlying metabolic causes of symptoms.

As used herein, “disease”, “disorder”, “condition” and the like, as they relate to a subject's health, are used interchangeably and have meanings ascribed to each and all of such terms.

The present invention relates to the treatment of subjects, or patients, in need thereof. By “patient” or “subject in need” it is meant any organism who may be affected by the above-mentioned conditions, and to whom the treatment methods herein described are desired, including humans, domestic and non-domestic mammals such as canine and feline subjects, bovine, simian, equine and murine subjects, rodents, domestic birds, aquaculture, fish and exotic aquarium fish. It should be appreciated that the treated subject may be also any reptile or zoo animal. More specifically, the methods and compositions of the invention are intended for mammals. By “mammalian subject” is meant any mammal for which the proposed therapy is desired, including human, equine, canine, and feline subjects, most specifically humans. It should be noted that specifically in cases of non-human subjects, the method of the invention may be performed using administration via injection, drinking water, feed, spraying, oral gavage and directly into the digestive tract of subjects in need thereof. It should be further noted that particularly in case of human subject, administering of the compositions of the invention to the patient includes both self-administration and administration to the patient by another person.

The present invention discloses the identification of novel peptides that inhibit A3G cytidine deaminase activity, thereby efficiently preventing DSB repair processes mediated by A3G.

Thus, according to another aspect, the invention provides an isolated peptide comprising at least one of: (a) an amino acid sequence of at least one of Val¹-Lys²-His³-His⁴, as denoted by SEQ ID NO. 66, Lys¹-Gly²-Trp³-Phe⁴ as denoted by SEQ ID NO. 68 and X¹-Leu²-Tyr³-Tyr⁴-Phe⁵ as denoted by SEQ ID NO. 67. In certain embodiments, X₁ may be a positively charged amino acid selected from His and Arg. It should be noted that these peptides were derived from any one of HIV-1 viral infectivity factor (Vif) and APOBEC3F (A3F). In yet other embodiments, the inhibitory peptides of the invention may comprise (b) an amino acid sequence derived from residues 211-240 of A3G.

In other embodiments, the peptides provided by the invention comprise an amino acid sequence of any one of residues 25-39, 105-119 and 107-115 of HIV-1 Vif, residues 304-312, 305-311 and 224-231 of A3F, residues 226-240, 211-225 and 226-240 of A3G, or any fragments, derivatives, homologues, or any combination thereof.

Certain embodiments of the invention relate to peptides comprising an amino acid sequence of any one of residues 25-39, 105-119 and 107-115 of HIV-1 Vif, as denoted by SEQ ID NO. 7, 27 and 71, respectively, residues 304-312 and 305-311 of A3F as denoted by SEQ ID NO. 74 and 75, respectively and residues 211-225 and 226-240 of A3G as denoted by SEQ ID NO. 83 and 84, respectively, or any fragments, derivatives, homologues, or any combination thereof.

In one specific embodiment the invention provides a peptide comprising the amino acid sequence of residues 25-39 of HIV-1 Vif having the amino acid sequence of VKHHMYISGKAKGWF as denoted by SEQ ID NO.:7, or any derivatives, homologues, or any combination thereof. In yet another specific embodiment, the invention provides a peptide comprising the amino acid sequence of residues 105-119 of HIV-1 Vif that has the amino acid sequence of QLIHLYYFDCFSESA as denoted by SEQ ID NO.:27, or any fragments, derivatives, homologues, or any combination thereof. In some specific embodiments, the peptide of the invention is derived from residues 107-115 of HIV-1 Vif and has the amino acid sequence of IHLYYFDCF as denoted by SEQ ID. NO. 71. In another embodiment, the peptide of the invention is derived from residues 304-312 of A3F, and has the amino acid sequence of ARLYYFWDT as denoted by SEQ ID. NO. 74. In a further embodiment, the peptide of the invention is derived from residues 305-311 of A3F and has the amino acid sequence of RLYYFWD as denoted by SEQ ID. NO. 75. In a further embodiment, the peptide of the invention is derived from residues 211-225 of A3G and has the amino acid sequence of WVRGRHETYLCYEVE as denoted by SEQ ID. NO. 83.

In a further embodiment, the peptide of the invention is derived from residues 226-240 of A3G and has the amino acid sequence of RMHNDTWVLLNQRRG as denoted by SEQ ID. NO. 84.

The term “polypeptide” as used herein refers to amino acid residues, connected by peptide bonds. A polypeptide sequence is generally reported from the N-terminal end containing free amino group to the C-terminal end containing free carboxyl group.

More specifically, “Amino acid molecule”, “Amino acid sequence” or “peptide sequence” is the order in which amino acid residues connected by peptide bonds, lie in the chain in peptides and proteins. The sequence is generally reported from the N-terminal end containing free amino group to the C-terminal end containing amide Amino acid sequence is often called peptide, protein sequence if it represents the primary structure of a protein, however one must discern between the terms “Amino acid sequence” or “peptide sequence” and “protein”, since a protein is defined as an amino acid sequence folded into a specific three-dimensional configuration and that had typically undergone post-translational modifications, such as phosphorylation, acetylation, glycosylation, manosylation, amidati on, carboxylation, sulfhydryl bond formation, cleavage and the like.

Amino acids, as used herein refer to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. “Amino acid analogs” refers to compounds that have the same fundamental chemical structure as a naturally occurring amino acid, i.e., an alpha carbon that is bound to a hydrogen, a carboxyl group, an amino group, and an R group, e.g., homoserine, norleucine, methionine sulfoxide, methionine methyl sulfonium. Such analogs have modified R groups or modified peptide backbones, but retain the same basic chemical structure as a naturally occurring amino acid. “Amino acid mimetics” refers to chemical compounds that have a structure that is different from the general chemical structure of an amino acid, but that functions in a manner similar to a naturally occurring amino acid. Amino acids may be referred to herein by either their commonly known three letter symbols or by the one-letter symbols recommended by the IUPAC-IUB Biochemical Nomenclature Commission.

It should be noted that in addition to any of the peptides described herein, the invention further encompasses any derivatives, analogues, variants or homologues of any of the peptides. The term “derivative” is used to define amino acid sequences (polypeptide), with any insertions, deletions, substitutions and modifications to the amino acid sequences (polypeptide) that do not alter the activity of the original polypeptides. By the term “derivative” it is also referred to homologues, variants and analogues thereof, as well as covalent modifications of a polypeptides made according to the present invention.

It should be noted that the polypeptides according to the invention can be produced synthetically, or by recombinant DNA technology. Methods for producing polypeptides peptides are well known in the art.

In some embodiments, derivatives include, but are not limited to, polypeptides that differ in one or more amino acids in their overall sequence from the polypeptides defined herein, polypeptides that have deletions, substitutions, inversions or additions.

In some embodiments, derivatives refer to polypeptides, which differ from the polypeptides specifically defined in the present invention by insertions of amino acid residues. It should be appreciated that by the terms “insertions” or “deletions”, as used herein it is meant any addition or deletion, respectively, of amino acid residues to the polypeptides used by the invention, of between 1 to 50 amino acid residues, between 20 to 1 amino acid residues, and specifically, between 1 to 10 amino acid residues. More particularly, insertions or deletions may be of any one of 1, 2, 3, 4, 5, 6, 7, 8, 9, or 10 amino acids. It should be noted that the insertions or deletions encompassed by the invention may occure in any position of the modified peptide, as well as in any of the N′ or C′ termini thereof.

The peptides of the invention may all be positively charged, negatively charged or neutral. In addition, they may be in the form of a dimer, a multimer or in a constrained conformation, which can be attained by internal bridges, short-range cyclizations, extension or other chemical modifications.

The polypeptides of the invention can be coupled (conjugated) through any of their residues to another peptide or agent. For example, the polypeptides of the invention can be coupled through their N-terminus to a lauryl-cysteine (LC) residue andor through their C-terminus to a cysteine (C) residue.

Further, the peptides may be extended at the N-terminus andor C-terminus thereof with various identical or different amino acid residues. As an example for such extension, the peptide may be extended at the N-terminus andor C-terminus thereof with identical or different amino acid residues, which may be naturally occurring or synthetic amino acid residues. An example for such an extension may be provided by peptides extended both at the N-terminus andor C-terminus thereof with a cysteine residue. Naturally, such an extension may lead to a constrained conformation due to Cys-Cys cyclization resulting from the formation of a disulfide bond. Another example may be the incorporation of an N-terminal lysyl-palmitoyl tail, the lysine serving as linker and the palmitic acid as a hydrophobic anchor. In addition, the peptides may be extended by aromatic amino acid residues, which may be naturally occurring or synthetic amino acid residues, for example, a specific aromatic amino acid residue may be tryptophan. The peptides may be extended at the N-terminus andor C-terminus thereof with various identical or different organic moieties, which are not naturally occurring or synthetic amino acids. As an example for such extension, the peptide may be extended at the N-terminus andor C-terminus thereof with an N-acetyl group.

For every single peptide sequence defined by the invention and disclosed herein, this invention includes the corresponding retro-inverse sequence wherein the direction of the peptide chain has been inverted and wherein all the amino acids belong to the D-series.

The invention also encompasses any homologues of the polypeptides specifically defined by their amino acid sequence according to the invention. The term “homologues” is used to define amino acid sequences (polypeptide) which maintain a minimal homology to the amino acid sequences defined by the invention, e.g. preferably have at least about 65%, more preferably at least about 75%, even more preferably at least about 85%, most preferably at least about 95% overall sequence homology with the amino acid sequence of any of the polypeptide as structurally defined above, e.g. of a specified sequence, more specifically, an amino acid sequence of the polypeptides as denoted by any one of SEQ ID NO. 1-46 and 66-87.

More specifically, “Homology” with respect to a native polypeptide and its functional derivative is defined herein as the percentage of amino acid residues in the candidate sequence that are identical with the residues of a corresponding native polypeptide, after aligning the sequences and introducing gaps, if necessary, to achieve the maximum percent homology, and not considering any conservative substitutions as part of the sequence identity. Neither N- nor C-terminal extensions nor insertions or deletions shall be construed as reducing identity or homology. Methods and computer programs for the alignment are well known in the art.

In some embodiments, the present invention also encompasses polypeptides which are variants of, or analogues to, the polypeptides specifically defined in the invention by their amino acid sequence. With respect to amino acid sequences, one of skill will recognize that individual substitutions, deletions or additions to peptide, polypeptide, or protein sequence thereby altering, adding or deleting a single amino acid or a small percentage of amino acids in the encoded sequence is a “conservatively modified variant”, where the alteration results in the substitution of an amino acid with a chemically similar amino acid.

Conservative substitution tables providing functionally similar amino acids are well known in the art. Such conservatively modified variants are in addition to and do not exclude polymorphic variants, interspecies homologues, and alleles and analogous peptides of the invention.

For example, substitutions may be made wherein an aliphatic amino acid (G, A, I, L, or V) is substituted with another member of the group, or substitution such as the substitution of one polar residue for another, such as arginine for lysine, glutamic for aspartic acid, or glutamine for asparagine. Each of the following eight groups contains other exemplary amino acids that are conservative substitutions for one another:

1) Alanine (A), Glycine (G);

2) Aspartic acid (D), Glutamic acid (E);

3) Asparagine (N), Glutamine (Q);

4) Arginine (R), Lysine (K);

5) Isoleucine (I), Leucine (L), Methionine (M), Valine (V);

6) Phenylalanine (F), Tyrosine (Y), Tryptophan (W);

7) Serine (S), Threonine (T); and

8) Cysteine (C), Methionine (M)

More specifically, amino acid “substitutions” are the result of replacing one amino acid with another amino acid having similar structural andor chemical properties, i.e., conservative amino acid replacements Amino acid substitutions may be made on the basis of similarity in polarity, charge, solubility, hydrophobicity, hydrophilicity, andor the amphipathic nature of the residues involved. For example, nonpolar “hydrophobic” amino acids are selected from the group consisting of Valine (V), Isoleucine (I), Leucine (L), Methionine (M), Phenylalanine (F), Tryptophan (W), Cysteine (C), Alanine (A), Tyrosine (Y), Histidine (H), Threonine (T), Serine (S), Proline (P), Glycine (G), Arginine (R) and Lysine (K); “polar” amino acids are selected from the group consisting of Arginine (R), Lysine (K), Aspartic acid (D), Glutamic acid (E), Asparagine (N), Glutamine (Q); “positively charged” amino acids are selected form the group consisting of Arginine (R), Lysine (K) and Histidine (H) and wherein “acidic” amino acids are selected from the group consisting of Aspartic acid (D), Asparagine (N), Glutamic acid (E) and Glutamine (Q).

The derivatives of any of the polypeptides according to the present invention, e.g. of a specified sequence of any one of the polypeptides of SEQ ID NO. 1-46 and 66-87, may vary in their size and may comprise the full length polypeptide or any fragment thereof.

In certain embodiments the peptide compounds of the invention may comprise one or more amino acid residue surrogate. An “amino acid residue surrogate” as herein defined is an amino acid residue or peptide employed to produce mimetics of critical function domains of peptides.

When referring to peptidomimetics, what is meant is a compound that mimics the conformation and desirable features of a particular natural peptide but avoids the undesirable features, e.g., flexibility and bond breakdown. From chemical point of view, peptidomimetics can have a structure without any peptide bonds, nevertheless, the compound is peptidomimetic due to its chemical properties and not due to chemical structure. Peptidoinimetics (both peptide and non-peptidyl analogues) may have improved properties (e.g., decreased proteolysis, increased retention or increased bioavailability). It should be noted that peptidomimetics may or may not have similar two-dimensional chemical structures, but share common three-dimensional structural features and geometry. Each peptidomimetic may further have one or more unique additional binding elements.

For implementing the methods provided herein, particularly methods involving the inhibition of DSB repair processes by inhibition of APOBEC3 expression andor activity, the invention further provides novel peptides performing a marked inhibitory effect on APOBAC3. As demonstrated by Example 16, the peptides of the invention were also applicable in preventing in vivo DSB repair processes. Therefore, another aspect of the invention concerns a composition comprising at least one peptide comprising: (a) an amino acid sequence of at least one of Val¹-Lys²-His³-His⁴, as denoted by SEQ ID NO. 66, Lys¹-Gly²-Trp³-Phe⁴ as denoted by SEQ ID NO. 68 and X¹-Leu²-Tyr³-Tyr⁴-Phe⁵ as denoted by SEQ ID NO. 67. In certain embodiments, X₁ may be a positively charged amino acid selected from His and Arg. It should be noted that these peptides were derived from any one of HIV-1 viral infectivity factor (Vif) and APOBEC3F (A3F). In yet other embodiments, the inhibitory peptides of the invention may comprise (b) an amino acid sequence derived from residues 211-240 of A3G as denoted by SEQ ID NO. 86.

Some peptides are considered more efficient than others for the purposes of the invention. Accordingly, certain embodiments of the invention relate to compositions comprising peptides comprising an amino acid sequence of any one of residues 25-39, 105-119 and 107-115 of HIV-1 Vif, as denoted by SEQ ID NO. 7, 27 and 71, respectively, residues 304-312 and 305-311 of A3F as denoted by SEQ ID NO. 74 and 75, respectively and residues 211-225 and 226-240 of A3G as denoted by SEQ ID NO. 83 and 84, respectively, or any fragments, derivatives, homologues, or any combination thereof.

It should be noted that the compositions provided by the invention may comprise any of the peptides described by the invention. It should be further appreciated that the invention encompasses any specific composition comprising any combination of all or part of the peptides of SEQ ID NO.:7 27, 66, 67, 68, 83 and 84, or any other combination of the peptides described herein is also contemplated by the invention.

According to an optional embodiment, the composition of the invention may further comprise an additional therapeutic agent, such as RPA that was shown by the invention as having an inhibitory effect on A3G.

In yet another embodiment, the composition of the invention may comprise as an additional therapeutic agent at least one a genotoxic-insult inducing agent.

According to one embodiment, the composition of the invention may be used as a pharmaceutical composition for treating, inhibiting, preventing, ameliorating or delaying the onset of a pathological disorder in a subject in need thereof by reducing, inhibiting or attenuating the activity of at least one APOBEC family member. The composition optionally further comprises a pharmaceutically acceptable excipient or carrier.

In some embodiments, the cytidine deaminase activity of APOBEC3 is inhibited by the composition of the invention, thereby preventing DSB repair processes. In certain embodiments such inhibition may be particularly applicable in treating proliferative disorders and specifically, lymphoma. In yet another embodiment, the composition of the invention may be suitable for treating any genotoxic-drug resistant cancer.

In certain embodiments, the composition of the invention, by inhibiting deaminase activity of APOBEC3, may prevent DSB repair processes, and therefore, may be use for sensitizing malignant cells to genotoxic inducing treatment (chemotherapy or irradiation). According to such embodiments, the composition of the invention may be used as an adjuvant cancer therapy. It should be noted therefore that according to certain embodiments, the composition may be adapted for use before, simultaneously with, after or any combination thereof, said genotoxic treatment.

More specifically, the compositions of the present invention can be administered for prophylactic andor therapeutic treatments. In therapeutic application, compositions are administered to a patient already affected by a proliferative disorder (e.g., lymphoma) in an amount sufficient to cure or at least partially arrest the condition and its complications. An amount adequate to accomplish this is defined as a “therapeutically effective dose.” Amounts effective for this use will depend upon the severity of the condition and the general state of the patient's own immune system, but generally range from about 0.001 to about 1000 mg/Kg. Single or multiple administrations on a daily, weekly or monthly schedule can be carried out with dose levels and pattern being selected by the treating physician. Additionally, the administration of the compositions of the invention, may be periodic, for example, the periodic administration may be effected twice daily, three time daily, or at least one daily for at least about three days to three months. The advantages of lower doses are evident to those of skill in the art. These include, inter alia, a lower risk of side effects, especially in long-term use, and a lower risk of the patients becoming desensitized to the treatment.

In another embodiment, treatment using the compositions of the invention, may be effected following at least 1, 2, 3, 4, 5, 6, 7, 8, 9, 10, 14, 30, 60, 90 days of treatment, and proceeding on to treatment for life. It should be noted that the treatment of different conditions may indicate the use of different doses or different time periods; these will be evident to the skilled medical practitioner.

The term “prophylactically effective amount” is intended to mean that amount of a pharmaceutical composition that will prevent or reduce the risk of occurrence or recurrence of the biological or medical event that is sought to be prevented in a tissue, a system, animal or human by a researcher, veterinarian, medical doctor or other clinician. In prophylactic applications, the compositions of the invention are administered to a patient who is at risk of developing the disease state to enhance the patient's resistance. Such an amount is defined to be a “prophylactically effective dose”. In this use, the precise amounts again depend upon the patient's state of health and general level of immunity, but generally range from 0.001 to 1000 mg per dose.

As mentioned herein before, the compositions provided by the invention optionally further comprise at least one pharmaceutically acceptable excipient or carrier. As used herein “pharmaceutically acceptable carrier” includes any and all solvents, dispersion media, coatings, antibacterial and antifungal agents and the like. The use of such media and agents for pharmaceutical active substances is well known in the art. Except as any conventional media or agent is incompatible with the active ingredient, its use in the therapeutic composition is contemplated.

Pharmaceutical compositions used to treat subjects in need thereof according to the invention generally comprise a buffering agent, an agent who adjusts the osmolarity thereof, and optionally, one or more pharmaceutically acceptable carriers, excipients andor additives as known in the art. Supplementary active ingredients can also be incorporated into the compositions. The carrier can be solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants.

The pharmaceutical composition of the invention can be administered and dosed by the methods of the invention, in accordance with good medical practice, systemically, for example by parenteral, e.g. intravenous, intraperitoneal or intramuscular injection. In another example, the pharmaceutical composition can be introduced to a site by any suitable route including intravenous, subcutaneous, transcutaneous, topical, intramuscular, intraarticular, subconjunctival, or mucosal, e.g. oral, rectal, vaginal, intranasal, or intraocular administration.

Local administration to the area in need of treatment may be achieved by, for example, local infusion during surgery, topical application, direct injection into the specific organ, etc.

More specifically, the compositions used in the methods and kits of the invention, described herein after, may be adapted for administration by parenteral, intraperitoneal, transdermal, oral (including buccal or sublingual), rectal, topical (including buccal or sublingual), vaginal, intranasal and any other appropriate routes. Such formulations may be prepared by any method known in the art of pharmacy, for example by bringing into association the active ingredient with the carrier(s) or excipient(s).

The pharmaceutical forms suitable for injection use include sterile aqueous solutions or dispersions and sterile powders for the extemporaneous preparation of sterile injectable solutions or dispersions. In all cases the form must be sterile and must be fluid to the extent that easy syringeability exists. It must be stable under the conditions of manufacture and storage and must be preserved against the contaminating action of microorganisms, such as bacteria and fungi.

The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with several of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above.

In the case of sterile powders for the preparation of the sterile injectable solutions, the preferred method of preparation are vacuum-drying and freeze drying techniques which yield a powder of the active ingredient plus any additional desired ingredient from a previously sterile-filtered solution thereof.

Compositions and formulations for oral administration include powders or granules, suspensions or solutions in water or non-aqueous media, capsules, sachets, lozenges (including liquid-filled), chews, multi- and nano-particulates, gels, solid solution, liposome, films, ovules, sprays or tablets. Thickeners, flavoring agents, diluents, emulsifiers, dispersing aids or binders may be desirable.

Pharmaceutical compositions used to treat subjects in need thereof according to the invention, which may conveniently be presented in unit dosage form, may be prepared according to conventional techniques well known in the pharmaceutical industry. Such techniques include the step of bringing into association the active ingredients with the pharmaceutical carrier(s) or excipient(s). In general formulations are prepared by uniformly and intimately bringing into association the active ingredients with liquid carriers or finely divided solid carriers or both, and then, if necessary, shaping the product. The compositions may be formulated into any of many possible dosage forms such as, but not limited to, tablets, capsules, liquid syrups, soft gels, suppositories, and enemas. The compositions of the present invention may also be formulated as suspensions in aqueous, non-aqueous or mixed media. Aqueous suspensions may further contain substances which increase the viscosity of the suspension including, for example, sodium carboxymethylcellulose, sorbitol andor dextran. The suspension may also contain stabilizers. The pharmaceutical compositions of the present invention also include, but are not limited to, emulsions and liposome-containing formulations.

It should be understood that in addition to the ingredients particularly mentioned above, the formulations may also include other agents conventional in the art having regard to the type of formulation in question, for example those suitable for oral administration may include flavoring agents.

Although the data presented by the invention support a positive role for ssDNA cytidine deamination in DSB repair, genomic mutagenesis induced by uncontrolled A3G activity may cause adverse cellular effects such as gene inactivation, chromosomal translocation and neoplasia. Notably, inherited DNA repair disorders which are associated with dysfunctional NHEJ often lead to development of lymphoid tumors. Thus, it is possible that uncontrolled A3G-directed mutagenesis andor DNA end joining might increase genomic instability and promote cancer under aberrant repair progression. In such case, inhibition of APOBEC deaminase activity may reduce genomic instability caused thereby and may therefore reduce the probability of developing a proliferative disorder. Therefore, in some embodiments, by inhibiting APOBEC deaminase activity, the compositions of the invention may prevent the mutagenic effect of APOBEC and thereby, may be used as a prophylactic treatment in preventing proliferative disorders or cancer progression.

The invention further provides a combined composition comprising at least one compound that modulates the expression or activity of at least one APOBEC family member, for example, at least one of the Vif polypeptide as well as Vif, A3F and A3G-derived peptides of the invention and at least one therapeutic agent, specifically an agent inducing genotoxic insult.

More specifically, in cases where the additional therapeutic agent is a genotoxic insult-inducing agent as a compound that inhibits at least one of the expression or activity of at least one APOBEC family member the combined composition of the invention may comprise any of the A3G inhibitors described by the invention. For example, such an inhibitor may be (I) a vif polypeptide or any fragment or peptide thereof, or any peptide derived from an APOBAC family member. More specifically, such an isolated peptide comprising any one of: (a) an amino acid sequence of at least one of Val¹-Lys²-His³-His⁴, as denoted by SEQ ID NO. 66, Lys¹-Gly²-Trp³-Phe⁴ as denoted by SEQ ID NO. 68 and X¹-Leu²-Tyr³-Tyr⁴-Phe⁵ as denoted by SEQ ID NO. 67. In certain embodiments, X₁ may be a positively charged amino acid selected from His and Arg. It should be noted that these peptides were derived from any one of HIV-1 viral infectivity factor (Vif) and APOBEC3F (A3F). In yet other embodiments, the inhibitory peptides of the invention may comprise (b), an amino acid sequence derived from residues 211-240 of A3G. It should be noted that any of the peptides of the invention, specifically the peptides of SEQ ID NO. 7, 27, 66, 67, 68, 83 and 84, may be applicable for the combined composition of the invention.

In yet another embodiment, such compound may be (II) at least one nucleic acid inhibitor specific for APOBEC, said inhibitor is any one of shRNA, siRNA, ribozyme or antisense RNA, or any functional fragments thereof, any combination thereof, or any vector comprising the same. Still further, the combined composition of the invention may comprise as an inhibitor of A3G, (III) a mutated A3G molecule devoid of cytidine deaminase activity, said mutant comprises at least one of W285A and E259Q point mutations or substitutions.

The present invention involves the use of different active ingredients, for example, the inhibitory Vif-derived peptides of the invention and at least one genotoxic insult inducing agent that may be administered through different routes, dosages and combinations. More specifically, the treatment of diseases and conditions with a combination of active ingredients may involve separate administration of each active ingredient. Therefore, a kit providing a convenient modular format of the different peptides and agents required for treatment would allow the required flexibility in the above parameters.

Thus, in another aspect, the invention provides a kit. In some embodiments the kit of the invention may includes at least two separate pharmaceutical compositions that are required for modulating a DSB repair process. According to certain embodiments, the kit of the invention may comprise (a) at least one compound that inhibits the expression or activity of at least one APOBEC family member, optionally, in a first unit dosage form; and (b) at least one genotoxic insult-inducing agent, and a pharmaceutically acceptable carrier or diluent, optionally, in a second unit dosage form.

For example, the kit of the invention is intended for modulating double stranded DNA breaks (DSB) repair processes in a subject in need thereof. In certain embodiments, the kit of the invention may comprise: at least one compound that inhibits the expression or activity of at least one APOBEC family member. For example, such an inhibitor may be (I) an isolated peptide comprising any one of: (a) a vif polypeptide or any fragment or peptide thereof, or any peptide derived from an APOBAC family member. More specifically, such peptide may comprise an amino acid sequence of any of the peptides of the invention. Specifically, the peptides of SEQ ID NO. 7, 27, 66, 67, 68, 83 and 84, may be applicable for the kit of the invention. In yet another embodiment, such compound may be (II) at least one nucleic acid inhibitor specific for APOBEC, specifically, the shRNA of the invention. Still further, as an inhibitor of A3G, the kit of the invention may comprise (III), a mutated A3G molecule devoid of cytidine deaminase activity. Specifically, at least one of W285A and E259Q mutant.

The kit of the invention may further comprise (b) at least one therapeutic agent, and a pharmaceutically acceptable carrier or diluent, optionally, in a second unit dosage form. In one particular embodiment, the additional therapeutic agent may be a genotoxic-insult inducing agent. In such case, the kit may be applicable in enhancing the sensitivity of the treated cancerous cells to said genotoxic-insult inducing agent.

It should be appreciated that in other embodiments, the therapeutic agent may be any agent suitable for ameliorating the treated disease. In such case, the kit of the invention may be suitable for preventing the mutagenic effect of A3G that may lead to development and progression of a malignant disorder.

According to some embodiments, the kit of the invention may further comprise container means for containing said first and second dosage forms. The term “container” as used herein refers to any receptacle capable of holding at least one component of a pharmaceutical composition of the invention. Such a container may be any jar, vial or box known to a person skilled in the art and may be made of any material suitable for the components contained therein and additionally suitable for short or long term storage under any kind of temperature. More specifically, the kit includes container means for containing separate compositions; such as a divided bottle or a divided foil packet however, the separate compositions may also be contained within a single, undivided container. Typically the kit includes directions for the administration of the separate components. The kit form is particularly advantageous when the separate components are preferably administered in different dosage forms (e.g., oral and parenteral), are administered at different dosage intervals, or when titration of the individual components of the combination is desired by the prescribing physician.

According to one embodiment, the kit of the invention is intended for achieving a therapeutic effect, specifically, modulating, or more specifically, inhibiting a DSB-repair process in a subject. In more specific embodiments, such subject may be a subject suffering from a proliferative disorder, particularly, a malignant disorder. The therapeutic effect may be for example slowing the progression of the treated condition. In more specific embodiments, where the additional therapeutic agent may be a genotoxic-insult inducing agent, the therapeutic effect may be manifested in increased sensitivity of the treated cancerous cells to said genotoxic-insult inducing agent (radiation or chemotherapy).

The invention further provides a method of treating, ameliorating, preventing or delaying the onset of a proliferative disorder in a subject in need thereof comprising the step of administering to said subject a therapeutically effective amount of the dosage unit forms comprised in a kit according to the invention. In certain embodiments, proliferative disease is a malignant proliferative disease. According to more specific embodiments, such proliferative disease may be lymphoma. In yet another embodiment, such proliferative disorder may be a genotoxic-drug-resistant cancer.

It should be appreciated that each of the multiple components of the kit may be administered simultaneously. Alternatively, each of said multiple dosage forms may be administered sequentially in either order. More specifically, the kits described herein can include a composition as described, or in separate multiple dosage unit forms, as an already prepared liquid topical, nasal or oral dosage form ready for administration or, alternatively, can include the composition as described as a solid pharmaceutical composition that can be reconstituted with a solvent to provide a liquid oral dosage form. When the kit includes a solid pharmaceutical composition that can be reconstituted with a solvent to provide a liquid dosage form (e.g., for oral administration), the kit may optionally include a reconstituting solvent. In this case, the constituting or reconstituting solvent is combined with the active ingredient to provide liquid oral dosage forms of each of the active ingredients or of a combination thereof. Typically, the active ingredients are soluble in so the solvent and forms a solution. The solvent can be, e.g., water, a non-aqueous liquid, or a combination of a non-aqueous component and an aqueous component. Suitable non-aqueous components include, but are not limited to oils, alcohols, such as ethanol, glycerin, and glycols, such as polyethylene glycol and propylene glycol. In some embodiments, the solvent is phosphate buffered saline (PBS).

Combining the identification of specific novel peptides derived from Vif, A3F and A3G, which inhibit the APOBEC deaminase activity, the invention further provides in another aspect, a method of treating, inhibiting, preventing, ameliorating or delaying the onset of a proliferative disorder in a subject in need thereof by inhibiting, reducing or attenuating the expression or cytidine deaminase activity of at least one APOBEC family member. As indicated above, reduction of A3G deaminase activity may reduce genomic mutagenesis and the incidence of developing a proliferative disorder. The method comprises the step of administering to the subject a therapeutically effective amount of at least one peptide as described by the invention. Specific and non-limiting embodiments refer to the use of the peptides as denoted by any one of SEQ ID NO. 7, 27, 71, 74, 75, 83 and 84 or any combination thereof.

In one embodiment, the method of the invention may use any of the peptides described herein before and any combinations thereof.

It should be noted that the mutagenic effect of APOBEC3 and the proliferative disorders caused thereby may be prevented also by using lentiviral Vif, either alone or in the context of lentiviral vector infection, as means to reduce A3G expression by proteasomic degradation andor to directly inhibit A3G deaminase activity.

It should be appreciated that any other compounds that inhibit, reduce or decease the activity andor expression of A3G are also applicable for this aspect. Non-limiting examples for such compounds include specific siRNA, ribozyme or anti-sense molecules that lead to reduction or elimination of A3G expression. Still further, the A3G mutants W285A and E259Q may be also used by the method of the invention.

In yet another aspect, the invention provides a method for treating, inhibiting, preventing, ameliorating or delaying the onset of a proliferative disorder in a subject in need thereof, wherein the subject is being treated with a genotoxic therapy. In certain embodiments, the method comprises the step of: administering to said subject a therapeutically effective amount of at least one compound that inhibits the expression or activity of at least one APOBEC family member. For example, such an inhibitor may be (I) a vif polypeptide or any fragment or peptide thereof, or any peptide derived from an APOBAC family member, as described by the invention. It should be noted that any of the peptides of the invention, specifically the peptides of SEQ ID NO. 7, 27, 66, 67, 68, 83 and 84, may be applicable for the combined composition of the invention.

In yet another embodiment, such compound may be (II) at least one nucleic acid inhibitor specific for APOBEC as described by the invention, specifically, the shRNA of SEQ ID NO. 92. Still further, A3G inhibitory compound used by the invention may be (III), a mutated A3G molecule devoid of cytidine deaminase activity, specifically, at least one of W285A and E259Q substitutions.

In yet another aspect, the invention further provides the use of a therapeutically effective amount of at least one isolated peptide in the preparation of a composition for the treatment of a proliferative disorder, specifically, drug resistance cancer. In certain embodiments the peptides may comprise at least one peptide as described by the invention. Specific and non-limiting embodiments refer to the use of the peptides as denoted by any one of SEQ ID NO. 7, 27, 71, 74, 75, 83 and 84 or any combination thereof.

In yet a further aspect, the invention provides the use of a therapeutically effective amount of at least one compound that inhibits the expression or activity of at least one APOBEC family member, in the preparation of a composition for the treatment of a proliferative disorder in a subject being treated with a genotixic therapy. For example, such an inhibitor may be (I) a vif polypeptide or any fragment or peptide thereof, or any peptide derived from an APOBAC family member, as described by the invention. It should be noted that any of the peptides of the invention, specifically the peptides of SEQ ID NO. 7, 27, 66, 67, 68, 83 and 84, may be applicable for the combined composition of the invention. In yet another embodiment, such compound may be (II) at least one nucleic acid inhibitor specific for APOBEC as described by the invention, specifically, the shRNA of SEQ ID NO. 92. Still further, A3G inhibitory compound used by the invention may be (III), a mutated A3G molecule devoid of cytidine deaminase activity, specifically, at least one of W285A and E259Q substitutions.

As shown in Example 6, and specifically in FIG. 7, the expression of A3G correlates with the ability of cancerous cells to repair DSB, and therefore reflects the resistance of these cells to treatment with genotoxic agents. As such, the invention provides a tool for efficiently evaluating the efficacy of a genotoxic treatment for a particular subject and therefore provides the use of A3G as a biomarker for predicting and evaluating the effect of a genotoxic therapeutic agent, specifically, chemotherapeutic drug, on a patient and thereby determining the efficacy of a suggested treatment on a particular patient.

Thus, another aspect of the invention relates to a method for determining the efficacy of a treatment with a genotixic therapy on a subject suffering from a proliferative disorder. In more specific embodiments the genotoxic therapy may be at least one of chemotherapeutic agent, irradiation or any combination thereof. More specifically, the method comprises the following steps:

The first step (a), involves determining the level of expression of at least one APOBEC family member in at least one biological sample of the examined subject, to obtain an expression value.

The next step (b) involves determining if the expression value obtained in step (a) is any one of, positive, negative or equal to a predetermined standard expression value (that is also referred to herein as a cutoff value) or to an expression value of at least one APOBEC family member in a control sample. Determination of a positive or negative expression value may be performed by comparing the expression value obtained in step (a) to a predetermined standard expression value or to an expression value of an APOBEC family member in a control sample. Such a step involves calculating and measuring the difference between the expression values of the examined sample and the cutoff value and determining whether the examined sample can be defined as positive or negative. More specifically, as used herein the term “comparing” denotes any examination of the expression level andor expression values obtained in the samples of the invention as detailed throughout in order to discover similarities or differences between at least two different samples. It should be noted that comparing according to the present invention encompasses the possibility to use a computer based approach. In certain embodiments, the method of the invention involves the use of an APOBEC family member that may be specifically, APOBAC3, more specifically, A3G.

It should be noted that in certain embodiments, a negative expression value of A3G in the tested sample indicates that the subject may responds to the genotoxic treatment and moreover, may exhibit a beneficial response to such treatment. More specifically, it should be noted that in certain embodiments, the predetermined standard values (cutoff values) are calculated and obtained from populations of subjects suffering from the same proliferative condition that responded well to the same genotoxic therapeutic agent, subjects not responding, healthy subjects and untreated subjects. Similarly, where control samples are used instead of, or in addition to predetermined cutoff values, such controls may include subjects suffering from the same proliferative condition that responded well to the same therapeutic agent, subjects not responding, healthy subjects and untreated subjects. Therefore, a negative expression value (when compared to cutoff representing the responder population), reflect low A3G expression, and indicates that the examined subject belongs to a pre-established population associated with a beneficial response to the specific genotoxic treatment that induces DSB. In contrast, a positive expression value, that is a result of enhanced or over-expression of A3G, indicates that the examined subject may not respond to said treatment and more specifically, may not exhibit a beneficial response to the genotoxic treatment. Thereby, the method of the invention provides determination of the efficacy of a specific genotoxic treatment on a specific subject that suffers from a proliferative condition.

The method of the invention is based on determining the expression level of a specific biomarker, A3G, in a sample. The terms “level of expression” or “expression level” are used interchangeably and generally refer to the amount of a polynucleotide or a protein in a biological sample. “Expression” generally refers to the process by which gene-encoded information is converted into the structures present and operating in the cell. Therefore, according to the invention “expression” of a gene, specifically, a gene encoding A3G may refer to transcription into a polynucleotide, translation into a protein, or even posttranslational modification of the protein. Fragments of the transcribed polynucleotide, the translated protein, or the post-translationally modified protein shall also be regarded as expressed whether they originate from a transcript generated by alternative splicing or a degraded transcript, or from a post-translational processing of the protein, e.g., by proteolysis. It should be noted that the expression level is reflected by measurement and determination of an expression value. As used herein, the term “expression value”, “level of expression” or “expression level” refers to numerical representation of a quantity of a gene product, which herein is a protein, but may also be an mRNA.

“Standard” or a “predetermined standard” as used herein, denotes either a single standard value or a plurality of standards with which the level of A3G expression from the tested sample is compared. The standards may be provided, for example, in the form of discrete numeric values or is calorimetric in the form of a chart with different colors or shadings for different levels of expression; or they may be provided in the form of a comparative curve prepared on the basis of such standards (standard curve). The standards may be prepared by determining the level of expression of A3G present in a sample obtained from a plurality of patients that were diagnosed or determined (by other means, for example by a physician, by histological techniques etc.) as performing a beneficial response (“responders”) to a certain treatment and a population of patients that do not respond well to the same therapeutic agent (non-responders, being correlated with a high level of expression of A3G). The level of expression for the preparation of the standards may also be determined by various conventional methods known in the art. The methods of the invention may be carried out in parallel to a number of standards of healthy subjects and subjects of different proliferative condition states that respond or not respond to a certain genotoxic treatment and the level determined in the assayed sample is then compared to such standards. After such standards are prepared, it is possible to compare the level of A3G expression obtained from a specific tested subject to the corresponding value of the standards, and thus obtain an assaying tool.

It should be noted that the term “response”, “responsiveness”, “responsive” or “responder” to treatment with a specific genotoxic agent refers to an improvement in at least one relevant clinical parameter as compared to an untreated subject diagnosed with the same pathology (e.g., the same type, stage, degree andor classification of the proliferative condition), or as compared to the clinical parameters of the same subject prior to said treatment.

The term “non responder” or “non-responsive” to treatment using a specific genotoxic agent, refers to a patient displaying resistance to a treatment, specifically, a patient not experiencing an improvement in at least one of the clinical parameter and is diagnosed with the same condition as an untreated subject diagnosed with the same pathology (e.g., the same type, stage, degree andor classification of the proliferative condition), or experiencing the clinical parameters of the same subject prior to such treatment. It should be noted that in certain embodiments, non-responder patients may particularly perform resistance to genotoxic treatment, and in certain embodiments may be perform a multi-drug resistance.

As used herein the phrase “predicting or evaluating efficacy of a treatment” refers to determining the likelihood that a specific treatment using a therapeutic agent is efficient or non-efficient in treating the proliferative condition, e.g., the success or failure of the treatment in treating the proliferative condition in a subject in need thereof. The term “efficacy” as used herein refers to the extent to which the genotoxic treatment produces a beneficial result, e.g., an improvement in one or more symptoms of the pathology (caused by the proliferative condition) andor clinical parameters related to the pathology.

In yet further specific embodiments of the invention, the determination of the level of expression of APOBAC in a biological sample of the tested subject may be performed by a method comprising the step of contacting detecting molecules specific for APOBAC with a biological sample of said subject, or with any nucleic acid or protein product obtained there from. It should be appreciated that determination of the level of A3G expression in the biological sample can be effected at the transcriptional level (i.e., mRNA) using detecting molecules that are based on nucleic acids (an oligonucleotide probe or primer), or alternatively, at the translational level (i.e. protein) using amino acid based detecting molecules, such as antibodies. Thus, according to one specific embodiment, the detecting molecules used by the method of the invention may be isolated detecting amino acid molecules or isolated detecting nucleic acid molecules, or any combinations thereof.

It should be noted that certain embodiments of the invention contemplate the use of different biological samples. The term “sample” in the present specification and claims is meant to include biological samples. Biological samples may be obtained from mammal, specifically, a human subject, include fluid, solid (e.g., stool) or tissues. The term “sample” may also include body fluids such as whole blood sample, blood cells, bone marrow, lymph fluid, serum, plasma, urine, sputum, saliva, faeces, semen, spinal fluid or CSF, the external secretions of the skin, respiratory, intestinal, and genitourinary tracts, tears, milk, any human organ or tissue, any biopsy, for example, lymph node or spleen biopsies, any sample taken from any tissue or tissue extract, any sample obtained by lavage optionally of the breast ductal system, plural effusion, samples of in vitro or ex vivo cell culture and cell culture constituents. Some samples that are a priori not liquid are contacted with a liquid buffers which are then used according to the diagnostic method of the invention.

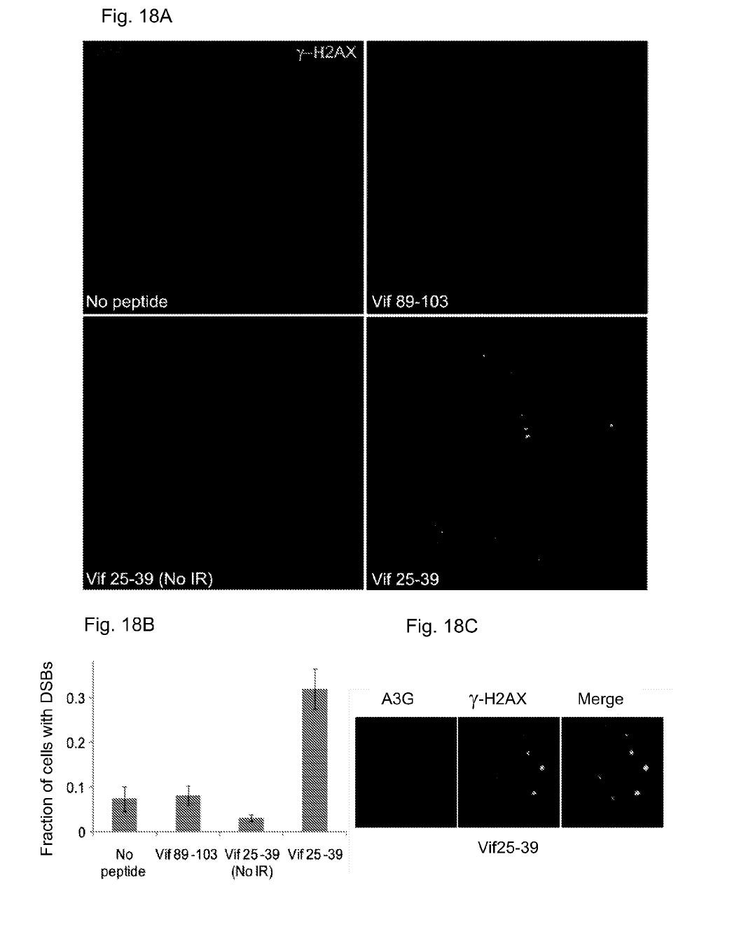

As shown in FIG. 18, compounds that inhibit the activity of A3G, specifically, the peptides of the invention may sensitize cells to a genotoxic treatment such as irradiation, therefore the invention further provides according to another aspect, a method for determining a genotoxic treatment regimen for a subject suffering from a proliferative disorder. The method of the invention comprises the steps of:

First (a), determining the level of expression of APOBEC3G (A3G) in at least one biological sample of said subject, to obtain an expression value.

In the second step (b), determining if the expression value obtained in step (a) is any one of, positive or negative with respect to a predetermined standard expression value or to an expression value of A3G in a control sample.

In certain embodiments a positive expression value of said A3G indicates that at least one compound that inhibits the expression or the activity of said A3G is required in addition to said genotoxic treatment for said subject.

In more specific embodiments, such an inhibitor may be (I) a vif polypeptide or any fragment or peptide thereof, or any peptide derived from an APOBAC family member, as described by the invention. It should be noted that any of the peptides of the invention, specifically the peptides of SEQ ID NO. 7, 27, 66, 67, 68, 83 and 84, may be applicable for the combined composition of the invention. In yet another embodiment, such compound may be (II) at least one nucleic acid inhibitor specific for APOBEC as described by the invention, specifically, the shRNA of SEQ ID NO. 92. Still further, A3G inhibitory compound used by the invention may be (III), a mutated A3G molecule devoid of cytidine deaminase activity, specifically, at least one of W285A and E259Q substitutions SEQ ID NO. 81 and 90). In addition to methods for treating DSB associated disorders by increasing DSB repair processes using A3G, any fragments thereof or any compound that enhances A3G expression andor activity, the invention further provides a composition for preventing DSB damage associated conditions, for example, conditions caused by exposure to radiation. Another aspect provided by invention therefore relates to a composition comprising as an active ingredient at least one APOBEC3 family member or any fragments thereof, optionally, combined with another DSB-repair enhancing molecule, andor with a molecule exhibiting regulatory effect on A3G, for example, at least one of Replication Protein A (RPA). This composition is effective for preventing or delaying the onset of double-strand DNA breaks (DSB)-associated condition, and optionally further comprises a pharmaceutical carrier, diluent, excipient andor additive.

In some embodiment, these compositions or combined compositions modulate cellular DSB repair, and in more specific embodiments, the modulation is the enhancement of cellular DSB repair.

As mentioned above, such DSB-repair enhancing composition may be relevant in treating ionizing radiation-induced DNA damage and associated conditions. For example, administration of A3G or any fragment thereof or any combination thereof with an additional therapeutic agent, or of any composition comprising the same, may be useful as a protective or therapeutic measure for workers handling radioactive material, or for military personnel and civilian population at risk of nuclear attack.

In yet another specific embodiment, such compositions may be applicable for treating DSB-related disorders including, but not limited to, Ataxia telangiectasia, Nijmegen breakage syndrome, Fanconi anemia and chromosomal translocations in general, which, in turn, may indeed lead to cancer and SCID. The invention also consider these compositions as well as methods thereof, for the treatment, amelioration, prevention or delaying the onset of Fragile-X syndrome.

In the last aspect, the invention relates to a kit for preventing or delaying the onset of a double-strand DNA breaks (DSB)-associated disorder in a subject, as well as conditions associated with ionizing radiation-induced DNA damage. In certain embodiments, such kit may comprise:

a. at least one APOBEC3 family member, optionally in a first unit dosage form, specifically, A3G as denoted by SEQ ID NO. 88; and optionally, at least one of:

b. at least one of Replication Protein A (RPA, specifically, as denoted by SEQ ID NO. 91) optionally in a first unit dosage form; and

c. optionally, container means for containing the at least one APOBEC3 family member and the at least one of Replication Protein A (RPA), or any combination or mixtures thereof dosage forms.

d. optionally, instructions for use of the kit.

In some embodiments, such kit may be used for treating ionizing radiation-induced DNA damage and associated conditions. Specifically, in cases of subjects handling radioactive material, or military personnel and civilian population at risk of nuclear attack.

In yet another specific embodiment, such kits may be applicable for treating DSB-related disorders including, but not limited to, Ataxia telangiectasia, Nijmegen breakage syndrome an Fanconi anemia.

Disclosed and described, it is to be understood that this invention is not limited to the particular examples, process steps, and materials disclosed herein as such process steps and materials may vary somewhat. It is also to be understood that the terminology used herein is used for the purpose of describing particular embodiments only and not intended to be limiting since the scope of the present invention will be limited only by the appended claims and equivalents thereof.

It must be noted that, as used in this specification and the appended claims, the singular forms “a”, “an” and “the” include plural referents unless the content clearly dictates otherwise.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

Unless defined otherwise, all technical and scientific terms used herein have the same meaning as commonly understood by one of ordinary skill in the art to which this invention is related. The following terms are defined for purposes of the invention as described herein.

The following Examples are representative of techniques employed by the inventors in carrying out aspects of the present invention. It should be appreciated that while these techniques are exemplary of preferred embodiments for the practice of the invention, those of skill in the art, in light of the present disclosure, will recognize that numerous modifications can be made without departing from the spirit and intended scope of the invention.

### EXAMPLES

Materials

Cells Culture

T-lymphoblastic leukemia (SupT1, SupT11, CEM-SS, MOLT-4), cutaneous T-cell lymphoma (H9, Hut78), multiple myeloma (ARH-77, NCI-H929, CAG), HL-60 acute myeloid leukemia, Ly-1 diffuse large B-cell lymphoma, and Raji Burkitt lymphoma cells were maintained at 1×10⁵ to 1×10⁶ mL in RPMI 1640 supplemented with 2 mM L-glutamine, 10% heat-inactivated FBS, 100 UmL penicillin and 0.1 mg/mL streptomycin (Beit-haemek) complete medium. Ly-4 diffuse large B-cell lymphoma cells were maintained in complete IMDM (Beit-haemek). SupT1 and H9 cells were provided by the National Institutes of Health AIDS Research and Reference Reagent Program (AIDSP; Division of AIDS, National Institute of Allergy and Infectious Diseases, National Institutes of Health) and Ly cells by D. Ben-Yehuda (Hadassah Medical School). PBMCs were donated by anonymous healthy volunteers after given consent and isolated on a Ficoll-Hypaque gradient (Sigma-Aldrich). Cells were maintained at 2×10⁶to 4×10⁶ mL in complete RPMI 1640. For induction of A3G expression, PBMCs were activated with phytohemagglutinin (5 μg/mL) for 36 hours, followed by supplement of IL-2 (20 UmL) for 36 hours [6] Human embryonic kidney 293T adherent cell lines were grown as a subconfluent monolayer in complete DMEM (Beit-Haemek).

HeLa-A3G cells stably transfected with pcDNA3.1-Apo3G expression vector encoding G418 resistance (obtained through the National Institutes of Health AIDSP) were grown in complete DMEM and with G418 (0.4 mg/mL; Invitrogen). U20S-DR-GFP cells (obtained from S. P. Jackson, University of Cambridge) were maintained in complete DMEM not containing phenol red, and containing charcoal-treated FBS.

Culture Media

RPMI 1640, DMEM, fetal calf serum, penicillin, streptomycin and L-glutamine were purchased from Biological Industries, Beit Haemek, Israel.

Reagents

BrdU (Invitrogen)

RNase A (Sigma-Aldrich)

PMSF (Sigma-Aldrich)

(Ni—NTA) agarose beads (QIAGEN)

Imperial protein stain (Pierce Biotechnology)

SYBR Gold (Molecular Probes, Invitrogen)

dCTP, dGTP, dTTP and dATP-biotin (Invitrogen)

IPTG (Sigma-Aldrich)

buffer S (Peqlab)

Eco 1471 (Fermentas)

Kits

QIAquick PCR purification kit (Qiagen)

HIV-1 p24 antigen capture assay kit (SAIC, AIDS Vaccine Program, Frederic, Md.)

Vectors

pLKO.1.shA3G (TRCN0000052188 clone ID: NM_{—}021822.1-398s1c1; Sigma-Aldrich)

pLKO.1.shCtr1 (Sigma-Aldrich)

pCMVΔR8.91 (Clontech)

Vesicular stomatitis virus G envelope protein (VSV-G) expression plasmid (Addgene)

pcDNA3.1-A3GMyc-His₆- (Invitrogen, gift from Dr Klaus Strebel)

pcDNA3.1-Apo3G expression vector encoding G418 resistance (obtained through the NIH AIDS Research and Reference Reagent Program from Drs. Klaus Strebel and Eri Miyagi)

pBlueScript SK+ plasmid (Stratagene)

pD10-Vif-His6 provided by Dr. Dana Gabuzda (Dana-Farber Cancer Institute, Boston, Mass.)

Antibodies

Rabbit polyclonal anti-A3G (NIH AIDS Research and Reference Reagent Program)

Mouse monoclonal anti-γ-H2AX (Cell Signaling, kindly provided by Dr. Eli Pikarsky)

Mouse monoclonal anti-RPA32 (Cell Signaling, kindly provided by Dr. Batsheva Kerem)

Goat anti-rabbit Cy-5-conjugated antibody (Jackson)

Goat anti-mouse Cy-2-conjugated antibody (Jackson)

Rabbit anti-Vif kindly provided by Dr. D. Gabuzda

Monoclonal anti-Ca-p24 kindly provided by Dr. B. Chesebro

Equipment

Zeiss LSM 710 confocal microscope

Zen software (Zeiss)

FACScan (Becton Dickinson Immunocytometry Systems)

Econo-column chromatography column (Bio-Rad Laboratories)

Multimode scanning probe microscope with a Nanoscope 3A controller (Digital Instruments/Veeco Probes)

Mica surfaces (Structure Probe)

NSC 15 AFM tips (Mikromasch)

WS×M SPIP software (Nanotec).

Olympus C-5050 CCD camera

TINA2.0 densitometry software (Raytest).

FPLC Akta (Amersham)

Experimental Procedures

shRNA-Mediated Knockdown of A3G

For short hairpin (sh) RNA expression, the following puromycin resistance-encoding vectors were used: A3G-specific pLKO.1.shA3G (TRCN0000052188, clone NM_{—}021822.1-398s1 c1; Sigma-Aldrich), having the nucleic acid sequence 5′-CCGG-GCCAGGTGTATTCCGAACTTA-CTCGAG-TAAGTTCGGAATACACCTGGC-TTTTTG-3′, as denoted by SEQ ID NO. 92, and the unrelated control cyprinid herpes virus 3 (CyHV3)-specific pLKO.1.shCtr1. Lentivectors were obtained by co-transfection of 293T cells with pLKO.1.shA3G or pLKO.1.shCtr1, the packaging plasmid pCMVΔR8.91 and vesicular stomatitis virus G envelope protein (VSV-G) expression plasmid. Culture supernatants were collected 48 h post transfection, centrifuged for 10 min at 4,000 rpm to remove cell debris and then for 1 h at 35,000 rpm using a swing SW-41 rotor (Beckman). H9 cells were transduced with the concentrated vectors by spinoculation for 1 h at 1,000 rpm and selected with puromycin (1 μg ml⁻¹) for 3-10 days post transduction.

Immunofluorescence Microscopy

Cells were irradiated by exposure to a ⁶⁰Co source producing 1 Gy sec⁻¹ γ-radiation, or mock-irradiated. Following incubation at 37° C., cells were fixed, attached to glass slides by cytospin, permeabilized with detergent and blocked with 10% normal goat serum. Cells were then incubated with A3G-specific rabbit polyclonal antibody, γ-H2AX-specific monoclonal antibody, or RPA32-specific monoclonal antibody, followed by incubation with goat anti-rabbit Cy-5-conjugated antibody, goat anti-mouse Cy-2-conjugated antibody and DAPI. Slides were mounted and examined by Zeiss LSM 710 confocal microscope. Data were collected sequentially using a ×63 objective with 7-fold averaging at a resolution of 102.4×1024 pixels, Z-sections were obtained using an optical slice of less than 1 μm. Data were analyzed with the Zen 2009.

Cell Cycle Analysis

PBMC, H9 or puromycin-resistant H9-shRNA cells were irradiated (4 Gy) or mock-irradiated. Following 20 h incubation at 37° C., cells were fixed with methanol for 1 h at −20° C., washed with phosphate buffered saline (PBS), treated with RNase A (50 μg ml ⁻¹) and stained with PI (5 μg ml⁻¹). The total cellular DNA content was determined by flow cytometry using FACScan. Cell cycle data were analyzed by the CellQuest Pro software and include 10,000 events gated on singlet cells.

Purification of Recombinant A3G Proteins

A3G and A3G W285A containing a C-terminal His₆ tag was expressed in 293Tcells and purified as previously described [8]. Briefly, 293T cells were transfected with pcDNA3.1-A3GMyc-His₆. Cells (3×10⁸) were harvested 48 h after transfection, washed three times in PBS and suspended in lysis buffer (50 mM Tris, pH 8.0, 1 mM PMSF, 10% (vv) glycerol and 0.8% (vv) NP-40), to a final concentration of 20,000 cells/μl. Following 10 min incubation in ice, cell debris and nuclei were pelleted by centrifugation at 10,000 g for 20 min. The soluble fraction was adjusted to 0.8 M NaCl and treated with 50 μg ml RNase A for 30 min at 37° C. Treated lysates were then added to 50 μl of nickelnitrilotriacetic acid (N—NTA) agarose beads, mixed on an end-over-end shaker for 1 h at 4° C. and loaded onto a chromatography column (Econo-column). Following extensive washing with wash buffer (50 mM Tris, pH 8.0, 0.3 M NaCl, 10% (vv) glycerol) containing 30-50 mM imidazole, bound proteins were eluted seven times in wash buffer containing 120 mM imidazole. Protein samples were resolved by SDS-PAGE and stained with Imperial protein stain. A3G concentration and purity was assessed by densitometry and scanning of stained gels, comparing band-intensity to that of a predetermined protein marker, and by a Bradford assay.

ssDNA Production

ssDNA (typically 1500-7000 nt) was produced by in-vitro rolling circle amplification as described [8].

Plasmid-Based DNA End Joining Assay

Preparation of HeLa and HeLa-A3G whole cell extracts (WCE) was performed as described before [7], with inclusion of RNase A treatment (50 μg ml⁻¹, Sigma) for 20 min at room temperature, following homogenization. DNA substrate was prepared by linearizing the pBlueScript SK+ plasmid (Stratagene) with EcoRI, EcoRI and PstI or EcoRV (Fermentas) to produce DNA fragments (3 kb) with compatible ends, non-compatible ends and blunt ends, respectively. Restriction reaction products were purified with QIAquick PCR purification kit (Qiagen). End joining assays were performed in 50 μl and contained 60 ng linearized plasmid, 0.3 μg WCE, 25 mM Tris (pH 7.5), 100 mM KCl, 5 mM MgCl₂, 5 mM DTT and 0.5 mM ATP. Control ligations with T4 DNA ligase (New England Biolabs) were performed in the designated buffer. Reactions were incubated for 18 h at room temperature (˜21° C.) and terminated by adding proteinase K (0.1 mg ml⁻¹). Following 1.5 h incubation at 50° C., DNA was extracted with phenolchloroform and ethanol-precipitated. DNA was resuspended in H₂O, separated by agarose (1.2%) gel electrophoresis and stained with SYBR Gold (Molecular Probes) as previously described [8]. Gels were visualized by UV light (302 nm), captured by an Olympus C-5050 CCD and analyzed by optical density (OD) scan using the TINA2.0 densitometry software (Raytest).

Analysis of Terminal ssDNA Tethering by DNA Polymerase Terminal Extension

DNA1 and DNA2 were annealed to an equimolar amount of a short oligonucleotide (30 nt) complementary to the 5′-termius by heating to 95° C. followed by slow cooling to RT. DNA-polymerase extension reactions were performed in a total volume of 7 μl and included 100 fmol annealed oligonucleotides, 50 μM dNTPs (dCTP, dGTP, dTTP and dATP-biotin (Invitrogen)), NEBuffer 1 (final dilution ×0.35 (NEB)), 1 mg/ml BSA, 100 fmol A3G or A3G W285A, and 5 u Klenow Fragment (3′→5′ exo(−), NEB). Reactions were incubated for 30 min at 37° C. and terminated by heating to 95° C. for 5 min and adding 5 μl formamide. Samples were then heated to 80° C. prior to loading on pre-heated 10% acrylamide gel containing 6M urea. Gels were washed in Tris-Boric acid-EDTA buffer (ph 8.3) for 15 min and transferred to nylon membranes as described in the EMSA protocol below.

Atomic Force Microscopy

AFM imaging was performed at room temperature using a multimode scanning probe microscope with a Nanoscope 3A controller. AFM images were recorded on freshly cleaved mica surfaces. The mica surfaces were activated with 5 mM MgCl for 1 min and washed with distilled water. For binding assays, purified A3G or RPA and ssDNA were incubated on ice in buffer containing 25 mM Tris (pH 7) in 10 μl reaction volume. Samples were deposited on the mica surface for 1.5 minutes, and washed with distilled water. Images were taken with NSC 15 AFM tips using the tapping mode at their resonant frequency. The images were analyzed with WSxM SPIP software. Stoichiometry of bound A3G proteins was calculated by the flooding option, assuming a monomeric volume of approximately 60 nm³.

Electrophoretic Mobility Shift Assay (EMSA)

S_{C} (10 pmol) was 3′ labeled with dATP-biotin using 10 u terminal deoxynucleotidyl transferase (NEB) in NEBuffer 4 (NEB) supplemented with 0.25 mM CoCl₂ and 10 μM dATP-biotin (Invitrogen). Labeled oligos (200 fmol) were incubated with A3G or RPA for 8 or 30 mM, respectively, at room temperature, in EMSA buffer: 50 mM Tris (pH 7.0), 50 mM NaCl, 0.1 mg/mL BSA and 10% glycerol, in 10 μL, reaction volume. Samples were resolved by 6% native PAGE, transferred to a Hybond N nylon membrane (GE Healthcare) using a semi-dry transfer apparatus (Biorad) and UV-crosslinked (302 nm) for 15 min. Following blocking with Casein blocking buffer (Sigma), the membrane was treated with horseradish peroxidase conjugated sterptavidine (Jackson) for 20 mM at RT, washed 6 times with TBS (pH 7.4) and visualized by enhanced chemiluminescence. Alternatively, complementary non-biotinylated oligonucleotides were annealed by heating to 95° C. and slow cooling to room temperature for 1 h. Following incubation with A3G, samples were resolved by PAGE, stained with SYBR Gold (Molecular Probes) and visualized by UV light (302 nm).

Preparation of Virus Stocks

Wild type HIV-1 and HIV-1 Δvif were generated by transfection of 293T cells with pSVC21 plasmid containing full length HIV-1HXB2 or Δvif viral DNA. Viruses were harvested 48 and 72 h post transfection and stored at −80° C. until infection of cultured H9 and SupT1 cells.

Infection of Cultured Cells

Cultured human T lymphoblastoid H9 and SupT1 cells (5×10⁶) were centrifuged for 5 mM at 500 g, the supernatant was aspirated and cells were re-suspended in 30 μl of medium containing 5-10 ng of p24 per ml of wt or HIV-1 Δvif: Cells were infected by spinoculation at 1200 rpm for 100 min at room temperature. Following infection, cells were re-suspended in fresh RPMI medium supplied with 10% FCS, and incubated for additional 7-10 days at 37° C. Culture media were harvested daily starting at 5 d.p.i. and viruses were titered by the multinuclear activation of a galactosidase indicator (MAGI) assay. The harvests containing 5×10⁴ to 10⁵ IU/ml were further centrifuged to remove cells and cell debris (10,000 g for 10 mM) and concentrated by ultracentrifuge for 1.5 h at 100,000 g through a 20% sucrose cushion. Pelleted virus was re-suspended in small volume of phosphate buffered saline (PBS) and stored at −80° C. until use.

HIV-1 Titration

HIV-1 was titered by the MAGI assay, as described by Kimpton and Emerman [Kimpton J, Emerman M, (1992) J. Virol. 66:2232-9].

Quantification of p24

HIV-1 p24 antigen capture assay kit was used to determine the amounts of p24 in the culture medium, according to the standards and instructions supplied by the manufacturers.

Expression and Purification of Vif

The pD10-Vif-His6 plasmid [9] was used to express an N-terminal His₆ tagged HIV-1HXBII Vif protein in E. coli MC-1061. Vif was purified as previously described [9], with the following exceptions: after induction of Vif expression with 0.5 mM IPTG for 1 h at 37° C., bacteria were pelleted at 4,000 g for 15 min, washed with PBS and suspended in lysis buffer containing 50 mM phosphate buffer (pH 8.0), 0.3 M NaCl, 25 μg/ml DNase, 1 mM PMSF, 5 mM imidazole and 0.8% NP-40. Following sonication, insoluble cell debris and inclusion bodies were removed by centrifugation at 10,000 g for 20 min and the soluble fraction was subjected to Ni²⁺ affinity chromatography. Briefly, 4 ml of the sample corresponding to 200 ml bacterial culture were incubated with 1 ml 50% Ni—NTA slurry for 1 h at 4° C. Following extensive washing in wash buffer [50 mM phosphate buffer (pH 8.0), 0.3 M NaCl] containing 10-40 mM imidazole, Vif was eluted in the same buffer containing 110 mM imidazole. The eluted sample was dialyzed against A3G reaction buffer for 6 h at 4° C. The concentration and purity of the Vif preparation were assessed as described above for A3G.

Western Blot

Transfected or infected cells were harvested, washed once in PBS, re-suspended in SDS-gel loading buffer and boiled for 10 min. Samples of 5×10⁴ cells were analyzed by SDS-12% polyacrylamide gel electrophoresis (SDS-PAGE), followed by transfer of the proteins to polyvinylidene fluoride (PVDF) membrane. HIV-1 proteins were detected by using rabbit anti-Vif and monoclonal anti-Ca-p24 antibodies. A3G protein was identified by rabbit polyclonal anti-A3G.

Deamination Assay

Deaminase activity of purified enzyme and virion-associated A3G protein was examined as previously described [8]. Briefly, A3G deamination reactions were performed in a total volume of 10 μl in 25 mM Tris, pH 7.0, and 0.01-1 fmol single-stranded (ss) deoxyoligonucleotide substrate at 37° C. The reaction was terminated by heating to 95° C. for 5 min. One μl of the reaction mixture was used for PCR amplification performed in 20 μl buffer S, using the following program: 1 cycle at 95° C. for 3 min, followed by 30 cycles of annealing at 61° C. for 30 s and denaturing at 94° C. for 30 s. Aliquots of the PCR products (10 μl) were incubated with Eco147I restriction enzyme for 1 h at 37° C. Completion of the restriction reaction was verified by using positive-control substrate containing CCU instead of CCC. Restriction-reaction products were loaded onto 14% gels and separated by PAGE. Gels were stained with SYBR gold nucleic acid stain diluted 1:10,000 in 0.5× Tris-Borate-EDTA buffer (TBE, pH 7.8), visualized by UV light (312 nm), captured by an Olympus C-5050 charge-coupled device (CCD) camera and analyzed by optical density (OD) scan using the TINA2.0 densitometry software. Assessment of A3G entrapped in virions was carried out with concentrated viruses stocks of 3-5 μg of p24/μl) suspended in PBS containing Triton X-100 at final concentration of 0.1% (vv) and 50 μg/ml RNase A (Sigma-Aldrich). Deamination reactions were incubated for 1 h at 37° C.

Inhibition of A3G Catalytic Activity

A panel of 46 HIV-1 Vif-derived 15-mer peptides (obtained through the NIH AIDS Research and Reference Reagent Program) were screened for inhibition of purified A3G-His₆ catalytic activity in a standard cytidine deamination assay. The inhibitory peptide Vif25-39 corresponds to amino acids 25-39 in HIV-1 HXB_{II} Vif. A fluorescein-conjugated peptide (kindly provided by Dr. Assaf Friedler) was used to assess the peptide uptake by H9 cells. For inhibition of endogenous A3G in H9 cells, cells were incubated with 100 μM Vif25-39 or a control peptide for 2 h at 37° C. before exposure to IR.

HR Assay

Repair of a unique ISceI-induced DSB by HR was assessed in U2OS-DR-GFP cells stably transfected with the Cherry-Sce-GR plasmid (HRind cells) as described in Shahar OD et al. [10]. Cherry-ZScel-GR rapidly translocates from the cytoplasm into the nucleus on binding to triamcinolone acetonide (TA). The DR-GFP cassette enables reconstitution of GFP after ZScel-dependent HR between 2 mutated GFP sequences [11]. Transfection of A3G or A3GW285A expression plasmids was performed in 24-well plates with GenJet (Signa-gen). Forty hours after transfection, IScel-directed DSB was induced with 10⁷M TA (Sigma-Aldrich). GFP⁺ cells were sorted 52 hours later by flow cytometry using FACScan as described in Shahar OD [10].

NHEJ Assay

Preparation of HeLa and HeLa-A3G whole-cell extracts (WCEs) was performed as described [7] with inclusion of RNase A treatment (50 μg/mL; Sigma-Aldrich) for 20 minutes at room temperature, after homogenization. DNA substrates were prepared by linearizing the pBlueScript SK+ plasmid (Stratagene) with EcoRI, ApaI, or EcoRV (Fermentas) to produce DNA fragments (3 kb) with compatible ends or blunt ends. Restriction reaction products were purified with QIAquick PCR purification kit (QIAGEN). End joining assays were performed in 50 μL and contained 60 ng linearized plasmid, 0.3 μg WCE, 25 mM Tris, pH 7.5, 100 mM KCl, 5 mM MgCl₂, 5 mM DTT, and 0.5 mM ATP. Control ligations with T4 DNAligase (NEB) were performed in the designated buffer. Reactions were incubated for 18 hours at room temperature (˜21° C.) and terminated by adding proteinase K (0.1 mg/mL) After 1.5-hour incubation at 50° C., DNA was extracted with phenolchloroform (1:1) and ethanol-precipitated. DNA was resuspended in H₂O, separated by agarose (1.2%) gel electrophoresis and stained with SYBR Gold (Invitrogen). Gels were visualized by UV light (302 nm), captured by an Olympus C-5050 CCD, and analyzed by optical density (OD) scan using the TINA2.0 densitometry software (Raytest).

Analysis of Terminal Cytidine Deamination in HIV-1 sssDNA

Wild type and vif⁽⁻⁾ HIV-1 were produced in H9 T cells endogenously expressing A3G, harvested and concentrated as described [8], and suspended in PBS. Endogenous reverse transcription reactions were performed in RPMI and contained viral particles equivalent to 10 ng p24 (CA) protein, 0.2 mM dNTPs, 10 mM MgCl₂, 10 mM Tris (pH 7.4), 20 μg ml⁻¹ BSA and 0.006% (vv) triton X-100. Following 3 h incubation at 37° C., samples were treated with 50 μg ml⁻¹ RNase A (Sigma) for 30 min at 37° C., DNA was extracted with phenol:chloroform (1:1) and purified using QIAquick PCR purification kit (Qiagen). SssDNA (approximately 0.02 fmol) was added to PCR containing template gDNA oligonucleotide (10 fmol), pFTag and pR604 primers (1 pmol), 0.2 mM dNTPs, buffer S and 0.2 u Taq polymerse (PeqLab), in 20 μl reaction volume, and subjected to the following PCR program: melting at 94° C. for 3 min, followed by 33 cycles of melting at 94° C. for 15 sec and polymerization at 64° C. for 40 sec. PCR products were resolved by PAGE (10%) and stained with SYBR Gold. Real time qPCR was performed with SYBR Green PCR Master Mix (Applied Biosystems) in ABI 7700 PCR machine (Applied Biosystems).

Inhibition of Reverse Transcription by Terminal Cytidine Deamination

S₅₁CCC (200 fmol) was incubated with A3G (200 fmol) in reaction buffer containing 25 mM Tris (pH 7) and 0.1 mg ml⁻¹ BSA, in 5 μl reaction volume. Following incubation for 1 h at 37° C., the oligonucleotide was annealed to the template S_{U3-R} (400 fmol) by heating to 95° C. followed by slow cooling to room temperature. S₅₁CCC was extended with recombinant HIV-1 reverse transcriptase (RT) enzyme (kindly provided by Dr. Amnon Hizi, Tel Aviv University, Israel), in a reaction containing the duplex oligonucleotides, RT (460 fmol), 25 mM Tris (pH 7.4), 50 mM KCl, 5 mM MgCl₂, 0.1 mM dNTPs and 1 mM DTT, in 6 μl reaction volume. Reactions were incubated for 30 mM at 37° C. and terminated by heating to 95° C. for 5 min and adding 5 μl formamide. Samples were then heated to 80° C. prior to loading on pre-heated 12% acrylamide gel containing 6M urea. Gels were washed in Tris-Boric acid-EDTA buffer (ph 8.3) for 15 min and transferred to nylon membranes as described in the EMSA protocol.

Synthetic Oligonucleotides and Peptides

Vif-derived peptides were obtained through the NIH AIDS Research and Reference Reagent Program as lyophilized powder and dissolved in water. Table 3 shows the amino acid sequences of the Vif derived peptides (SEQ ID NOs.: 1-46).

Oligonucleotides Were Obtained From IDTDNA, (Metabion).

The sequence of the 80-mer ss-deoxyoligonucleotide substrate used in the deamination assays is as denoted as SEQ ID NO. 47 (A3G target site is underlined and the preferentially deaminated dC residue is bold-face-type). The positive control ss-deoxyoligonucleotide bears the same sequence, but has a dU instead of the target dC residue. The following primers were used for PCR amplification of the substrate and positive control oligonucleotides: Forward primer is denoted as SEQ ID NO. 48; Reverse primer is denoted as SEQ ID NO. 49. S₁₆₀ as used herein is denoted by SEQ ID NO. 53; Complementary-to-5′ is denoted by SEQ ID NO. 54; Complementary-to-3′ is denoted by SEQ ID NO. 55; DNA1-biotin is denoted by SEQ ID NO. 56; DNA2 is denoted by SEQ ID NO. 57; S_{C} is denoted by SEQ ID NO. 58; S_{U3-R}—is denoted by SEQ ID NO. 59; gDNA —is denoted by SEQ ID NO. 60; pF509 is denoted by SEQ ID NO. 61; pR604 is denoted by SEQ ID NO. 62; pFTag is denoted by SEQ ID NO. 63; S₅₁CCC biotin is denoted by SEQ ID NO. 64; and S₅₁CUU biotin is denoted by SEQ ID NO. 65.

### Example 1

A3G Multimer Disassembly on ssDNA Termini

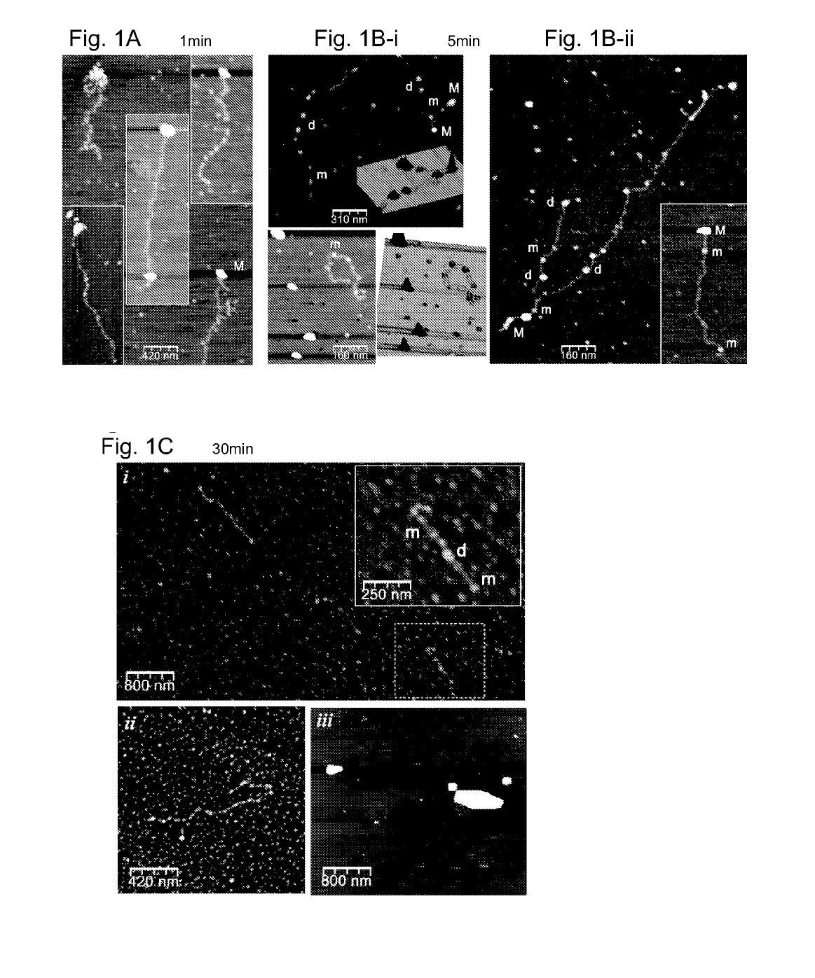

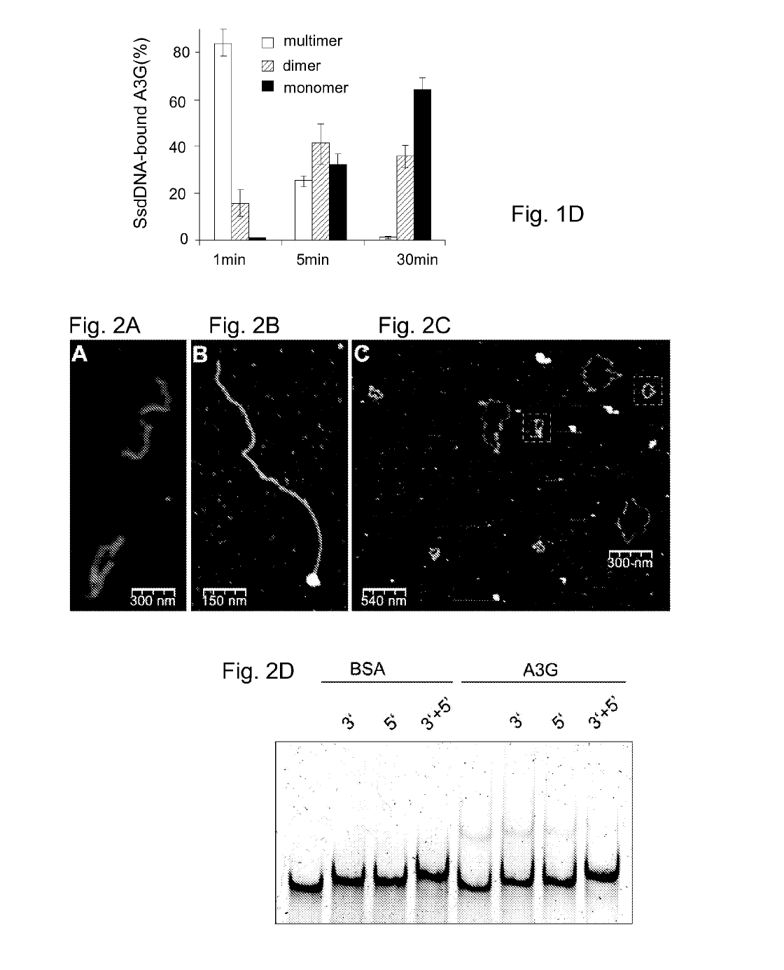

The inventors have previously shown that A3G-induced hypermutation is mediated by intersegmental transfer of A3G monomers on ssDNA [8]. Since the majority of human cell-derived A3G is multimeric (>90%), the inventors suggested that catalytically-inactive A3G multimers disassemble upon interaction with ssDNA [8]. To probe the dynamics of A3G interaction with ssDNA at the single-molecule level, about 1.5-7 (average=3) kb long ssDNA were generated by in-vitro rolling-circle amplification (RCA), and interactions of purified A3G-His₆ and purified ssDNA were assessed by AFM. Incubations were performed in ice in order to slow down A3G-DNA interaction. FIG. 1A shows that A3G multimers rapidly bind ssDNA preferentially at the DNA termini End-bound multimers either obscured the ssDNA terminus, and therefore presumably bound directly to the ssDNA terminus, or bound within tens to several hundreds of bases from the ssDNA terminus Following 5 min incubation, an overall reduction in the size of bound A3G multimers was observed, as well as A3G dimers and monomers occupying internal DNA domains, as can be seen in FIGS. 1B (i and ii). Following 30 min incubation, virtually all A3G multimers were reduced to monomers and dimers (FIG. 1C i and ii). FIG. 1C (iii) shows that this process was DNA-dependent, as in the absence of DNA, A3G remained in the form of high-order multimers. The fraction of ssDNA-associated multimeric, dimeric and monomeric A3G at each time point is summarized in FIG. 1D.

### Example 2

An ssDNA Terminus is Required for Multimeric A3G-ssDNA Interaction

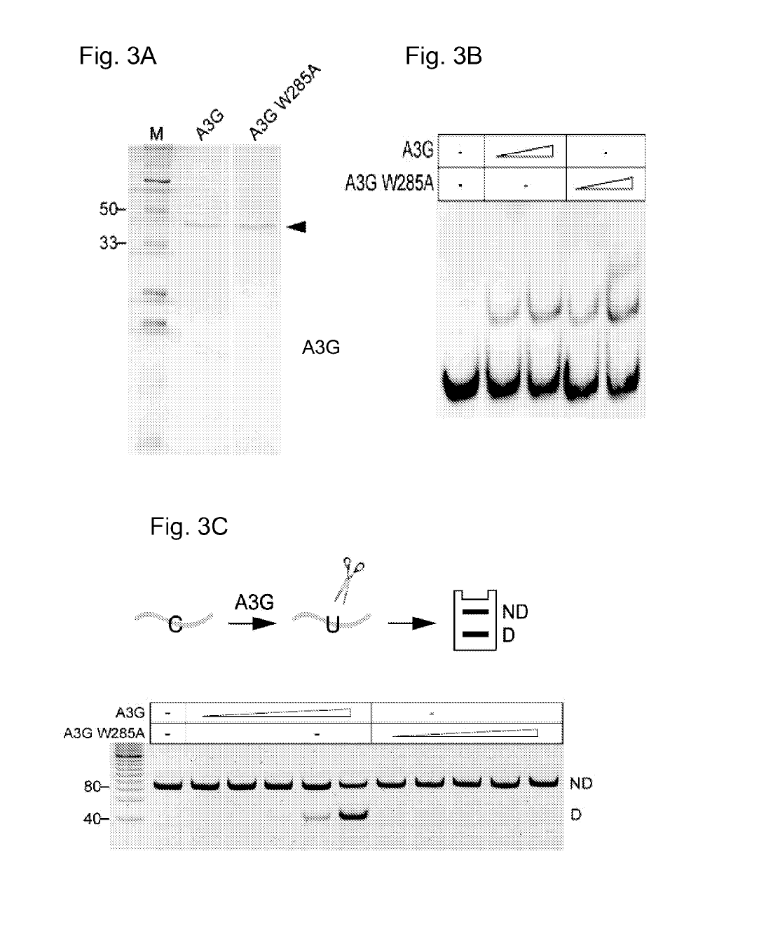

Assuming that A3G multimers require an ssDNA terminus for ssDNA binding, the inventors expected that A3G multimers will not readily bind circular ssDNA. To probe A3G interaction with circular ssDNA, the inventors used an M13 phage ssDNA sample which contained both linear and circular M13 genomic ssDNA, as illustrated in FIG. 2A. Similar to the RCA ssDNA, A3G multimers (the expression of which is shown in FIG. 3A) bound the linear M13 ssDNA termini following 5 min incubation (FIG. 2B). In contrast, A3G multimers apparently did not bind the circular M13 ssDNA following 5 min incubation (FIG. 2C). The requirement for an ssDNA terminus was further assessed by electro-mobility shift assay oligonucleotides (30 nt) at the 3′-, 5′- or both ends. FIG. 2D shows that A3G binding to S₁₆₀ was slightly reduced in the absence of 5′-ssDNA terminus, which is supported by previous observations regarding A3G 5′ preference [8]. However, A3G association with S₁₆₀ was markedly reduced when both termini were double-stranded. These results suggest that A3G multimers require either a 3′ or 5′ ssDNA terminus for functional association with ssDNA.

### Example 3

A3G deaminates the extreme base of the ssDNA 3′-terminus and tethers two ssDNA termini To determine whether A3G interacts with the terminal base of the ssDNA, the inventors tested terminal cytidine deamination by A3G. A primer with 3′-terminal CC was incubated with A3G and was then used for extension and PCR amplification of a target sequence. It was previously shown that A3G W285 residue contributes to formation of a hydrophobic cavity and is essential for catalytic activity. Since the W285 residue does not support the structure of A3G Z motif, the inventors assumed that a W285A mutant (as dented by SEQ ID NO. 81) will retain wild-type DNA binding properties and may serve as a deaminase-dead control. Indeed, A3G W285A retained wild-type ssDNA binding properties but was catalytically inactive, as determined by EMSA and cytidine deamination assay depicted in FIGS. 3B and 3C, respectively. More specifically, FIG. 3B shows that both wt A3G and A3G W285A bind biotinylated S_{C} oligonucleotides (80 nt) similarly. FIG. 3C (top) shows a scheme of the in vitro deamination assay. A cleavage resistant polynucleotide deaminase substrate, comprising an internal cytidine, was incubated with either A3G or A3G W285A. Upon deamination, the internal uridine-containing polynucleotide was susceptible to restriction, and yielded a fragment which was apparent in the PAGE analysis of the cleaved deamination products shown in FIG. 3C (bottom). FIG. 4A shows that incubation of the primer with A3G resulted in marked inhibition of the PCR reaction (˜90%), in contrast to normal PCR amplification in the presence of the A3G W285A catalytic mutant or recombinant ssDNA binding protein (SSB). Hence, A3G interacts with the extreme base of the ssDNA 3′-terminus and is able to perform terminal cytidine deamination.

A3G undergoes intersegmental transfer on ssDNA by tethering two separate ssDNA segments [8]. A3G ability to tether two ssDNA termini was assessed in a plasmid-based assay using whole cell extracts (WCE) of HeLa and HeLa-A3G cells. Specifically, the inventors measured joining efficiency of linearized plasmids with compatible ends (EcoRI-linearized), non-compatible ends (EcoRI and PstI-linearized) and blunt ends (EcoRV-linearized). HeLa WCE supported end joining of compatible DNA ends, promoting joining of two (×2) and three (×3) linear DNA molecules and utilizing approximately 17% of the substrate, as illustrated in FIG. 4B. However, the DNA end-joining activity of HeLa-A3G WCE was 4-5 fold more efficient, producing ×2, ×3 and higher forms of joint DNA (×4+), and utilizing over 80% of the substrate. Similar results were obtained with non-compatible ends, where substrate utilization of HeLa-A3G WCE was approximately 5 fold higher than HeLa WCE. Joining of blunt-ended substrate was less efficient in general, but was also enhanced by HeLa-A3G WCE.

To further investigate A3G-mediated terminal ssDNA tethering, the inventors developed a DNA polymerase extension assay depicted in FIG. 4C. The 5′-termini of two ss-oligonucleotides (DNA1 and DNA2, 80 nt) were annealed to complementary oligonucleotides (30 nt), leaving 3′ ssDNA overhangs. Only the two terminal bases in each overhang are complementary—GG in DNA2 and CC in DNA1—so that juxtaposing the two overhangs might enable each terminus to serve as a primer for DNA polymerase extension using the opposite strand as a template. In order to distinguish between the two extension products, biotinylated dATP were used in the polymerase reaction and the sequence of DNA1 was designed to contain less dT than DNA2. Thus, the DNA1 extension product which uses DNA2 as a template will contain more dATP-biotin compared to the DNA2 extension product, resulting in differential products migration in gel electrophoresis. Incubation of DNA1+2 with Klenow fragment yielded only background level products. However, a discernible extension product, which migrated slightly above the 160 nt marker, was apparent in the presence of A3G, as can be seen in FIG. 4D. Extension occurred approximately 9-fold more efficiently in the presence of A3G than with Klenow fragment alone. Interestingly, incubation with A3G W285A yielded a higher extension product with similar efficiency. Hence, it could be inferred that A3G mediated extension of DNA2 from the terminal GG, using DNA1 as a template, whereas the W285A catalytic mutant mediated extension of DNA1 from the terminal CC, using DNA2 as a template. The fact that A3G did not extend the CC terminus while the W285A mutant did, suggests that A3G deaminated the terminal cytidine, creating a U:G mismatch that cannot be utilized as a primer. To confirm that the extension products result from terminal ssDNA annealing, the terminal GG in DNA2 were substituted with TT, which cannot base-pair with the terminal CC of DNA1. As expected, no extension products were detected in the presence or absence of wild-type or mutant A3G (not shown). The inventors conclude that association of A3G with ssDNA termini promotes terminal cytidine deamination and tethering of ssDNA termini.

### Example 4

RPA Hinders A3G Multimer Disassembly and Activation

RPA is a ubiquitous nuclear heterotrimer required for DNA replication and involved in all major DNA repair pathways [12]. RPA binds non-specifically to ssDNA with an apparent association constant of 10⁻⁹-10⁻¹¹ M⁻¹ [12]. A3G also binds non-specifically to ssDNA with apparent dissociation constant of 5×10⁻⁸−7.5×10⁻⁸ M⁻¹ [5]. Analyses of RPA interaction with long ssDNA, illustrated in FIG. 5A, showed that RPA binds preferentially to the ssDNA termini or forms a V-shaped RPA-ssDNA complex when bound internally. To test whether RPA is involved in A3G association with ssDNA, A3G multimers were allowed to interact with ssDNA for one minutes before addition of RPA, followed by 29 min incubation. RPA inhibited A3G multimer disassembly, as the vast majority of DNA-bound and unbound proteins remained multimeric (FIG. 5B, compare to FIG. 1C) Inhibition of A3G multimer disassembly was presumably due to restricted accessibility of A3G to ssDNA termini occupied by RPA, or following formation of V-shaped RPA-ssDNA complexes. This was further verified by EMSA, in which RPA pre-bound to S_{C} ssDNA (80 nt) inhibited the formation of monomeric A3G-ssDNA complexes, shown in FIG. 5C. There was no indication of modulated DNA binding by RPA, A3G-RPA interaction or co-binding of the same DNA molecule, as RPA association with DNA was not affected by A3G. The inventors have previously shown that the catalytically active form of A3G is monomeric [8]. The data thus suggests that disassembly of A3G multimers observed in the presence of ssDNA (FIG. 1) therefore activates A3G catalysis. To validate RPA-mediated inhibition of A3G multimer disassembly, the inventors measured A3G enzymatic activity in the presence of RPA, assuming that inhibition of A3G monomerization will inhibit cytidine deamination. As shown in FIG. 5D, pre-incubation of ssDNA with RPA at sub-stoichiometric concentrations caused dose-dependent inhibition of A3G cytidine deaminase activity, reinforcing the functional link between A3G multimer disassembly and cytidine deamination.

### Example 5

A3G Targets Terminal ssDNA During HIV-1 Reverse Transcription

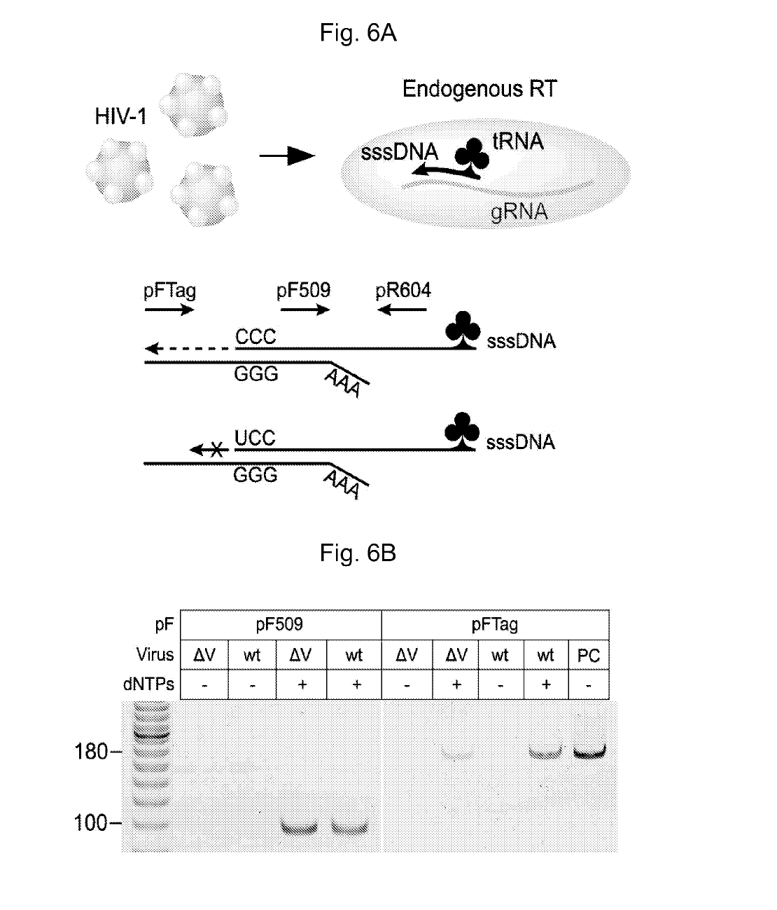

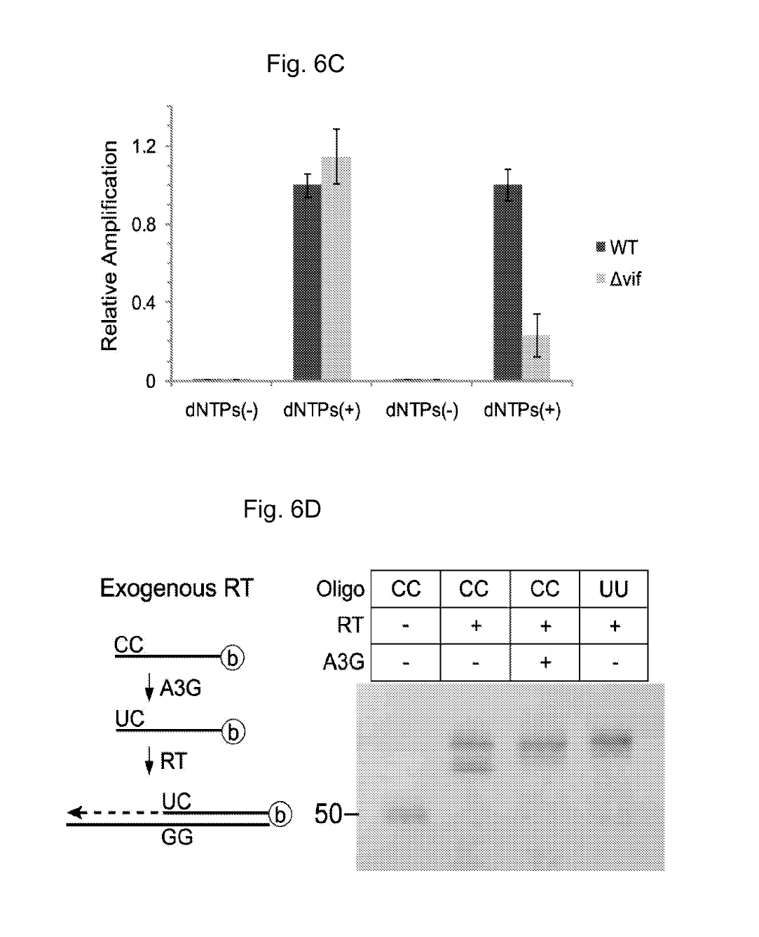

Following incorporation of A3G into viral particles in virus-producing cells, A3G restricts HIV-1 replication by deaminating cytidines in the viral ssDNA formed during reverse transcription. HIV-1 ssDNA synthesis by the viral reverse transcriptase (RT) initiates from a cellular tRNA primer annealed to the 5′ long terminal repeat (LTR) region of the viral RNA. FIG. 6A presents the first step of HIV-1 reverse transcription, i.e., formation of a short stretch of ssDNA of approximately 200 nt, referred to as strong-stop ssDNA (sssDNA). Since HIV-1 genomic RNA starts with GG or GGG from its 5′-terminus, the sssDNA synthesized on the genomic RNA ends with CC or CCC in its 3′-terminus, providing a potential A3G target site. This presented the opportunity to assess whether A3G interacts with terminal ssDNA inside HIV-1 virions, by measuring deamination of the terminal cytidine in the sssDNA. To assess terminal deamination, the inventors utilized endogenous reverse transcription in purified wt and vif⁽⁻⁾ HIV-1 virions produced in H9 T cells natively expressing A3G, and therefore have low and high A3G content, respectively [8]. sssDNA extracted from virions following reverse transcription was used in a primer extension assay, in which terminal cytidine deamination precludes extension of the sssDNA used as primer for Taq DNA polymerase (FIG. 6A). The extended primer was then used as a template for PCR amplification using a forward primer specific for the extension product (pFTag) and a reverse primer specific for the sssDNA (pR604). For controlling the input sssDNA content, the inventors used both forward and reverse primers specific for the sssDNA (pF509 and pR604). As shown in FIG. 6B, excluding dNTPs in the endogenous RT reaction resulted in no apparent PCR product in both the wt and vif⁽⁻⁾ viruses, indicating no background HIV-1 genomic RNA or DNA. PCR amplification of sssDNA from both the wt and vif⁽⁻⁾ viruses using the sssDNA-specific pF509 primer indicated comparable sssDNA input content. However, using the extension product-specific pFTag primer yielded 4-5 less PCR product of the expected length in case of sssDNA derived from vif⁽⁻⁾ virus (A3G high), compared to the wt virus (A3G low). This indicates that cytidine deamination at the extreme 3′-terminus of the sssDNA occurred in at least 75-80% of vif⁽⁻⁾ virions. Terminal deamination was verified by quantitative real-time PCR, corroborating a 4 fold decrease in PCR efficiency when using sssDNA from vif⁽⁻⁾ virions, as illustrated in FIG. 6C. It was previously shown that A3G inhibits HIV-1 reverse transcription. To determine whether terminal cytidine deamination in the sssDNA impedes reverse transcription, the inventors used purified HIV-1 RT in an exogenous reverse transcription assay, a scheme of which is provided in FIG. 6D (left). An oligonucleotide comprising the sequence of the sssDNA 3′-terminus (51 nt) was incubated with purified A3G and then used as a primer for RT extension in the presence of dNTPs. FIG. 6D (right) shows that incubation with A3G, or using a positive control oligonucleotide with 3′-terminal UU, did not prevent complete utilization and extension of the oligonucleotides by RT, indicating that terminal cytidine deamination does not inhibit HIV-1 reverse transcription. The inventors conclude that association of A3G with the sssDNA terminus during HIV-1 reverse transcription may promote A3G activation in vivo.

### Example 6

A3G Expression is Inversely Correlated With DSB Occurrence

The discovery that A3G can tether ssDNA (see FIG. 4B) and of the involvement of RPA, in regulating A3G deaminase activity, prompted the inventors to investigate the possible involvement of A3G in cellular DNA damage repair. DNA double strand breaks are considered the most lethal form of DNA damage for eukaryotic cells. DSB can either be properly repaired, restoring genomic integrity, or misrepaired resulting in drastic consequences, such as cell death, genomic instability, and cancer. It is well established that exposure to DSB-inducing agents is associated with chromosomal abnormalities and leukemogenesis. To explore the possible link between A3G and the response of lymphoma cells to genotoxic treatment, the inventors employed γ-radiation to generate DSBs in a panel of lymphocytic cell lines expressing differential A3G protein levels. More specifically, to assess the correlation, if any, between A3G expression and DSB occurrence, the inventors analyzed lysates of five different lymphoma and three leukemia cell lines for A3G expression, as depicted in FIG. 7A. The analysis demonstrated significant differences in A3G expression between the lines, with generally higher levels of A3G found in lymphoma cell lines. Phosphorylation of histone H2AX at specific sites of DNA DSBs occurs rapidly following DSB formation and is therefore indicative of the presence and location of DSBs. Thus, to determine the occurrence of DSBs, the inventors fixed and stained lymphoma and leukemia cell lines for γ-H2AX. As illustrated in FIG. 7B, lymphoma cell lines which express a relatively high level of A3G, such as H9 and Raji showed lower cellular DSB incidence following IR, encompassing ˜5-60% of cultured cells, inversely dependent on A3G expression level. Ly-4, a lymphoma cell line expressing a rather low A3G level displayed moderate DSB occurrence, whereas leukemic SupT1 or CEM (FIG. 7D) cells which expresses almost no A3G displayed a large number of DSBs. A plot of the fraction of cells with DSBs and the relative A3G expression level (fold from H9 A3G expression) presented in FIG. 7C clearly demonstrates an inverse relationship between A3G expression and DSB frequency. The high DSB incidence observed in leukemia cells persistently remained 24 h following IR, as presented by FIG. 7D. Thus, the degree of DSB repair in leukemia and lymphoma cell lines correlates with A3G expression level.

### Example 7

A3G is Recruited to the Nucleus Following DNA Damage and is Associated With DSBs

To investigate the role of A3G in the DNA damage response, the inventors probed A3G and γ-H2AX sub-cellular localization in H9 cells exposed to 4 Gy γ-radiation. As shown by FIG. 8A, A3G localizes predominantly to the cytoplasm of human peripheral blood mononuclear cells (PBMCs) and H9 T cells. To determine whether A3G is recruited to genomic DSBs, the inventors probed A3G and γ-H2AX sub-cellular localization in H9 cells exposed to 4 Gy γ-radiation. At 30 minutes following irradiation, A3G was more uniformly distributed throughout the cell, and multiple IR-induced DSBs were evident by the formation of γ-H2AX nuclear foci, as demonstrated by FIG. 8B. Remarkably, one hour following irradiation, A3G formed distinct nuclear foci which co-localized with γ-H2AX (FIGS. 8B and 8C). A3G accumulation at the breakage sites intensified 4 hours following irradiation, and coincided with reduction in the number and magnitude of γ-H2AX foci. Following 8 hours, A3G was again redistributed throughout the cell, with sporadic nuclear foci still evident at sites of minor γ-H2AX accumulation. These results suggest that A3G is involved in DSB repair. Consistently, A3G was also detected in 119 nuclear fractions 4 to 6 hours after IR, coinciding with a parallel reduction of A3G levels in the cytoplasm (FIG. 8D). These nuclear fractions were associated with cytidine deaminase activity measured on an oligunocleotide substrate (FIG. 8E). The reduction in cytoplasmic deaminase activity in the absence of comparable increase in nuclear activity 2 hours after IR may reflect sequestration of A3G activity by cytoplasmic RNA induced in the initial stage of the cellular response to IR. After peak nuclear deaminase activity 4 to 6 hours after IR, A3G activity was reduced in the nuclear fraction and increased in the cytoplasmic fraction 8 hours after IR to comparable levels as in nonirradiated cells, in line with A3G transient nuclear localization after IR. These results indicate that catalytically active A3G is transiently recruited to the nucleus and that A3G localizes to DSB repair foci in response to IR.

In case A3G is required for repair of IR-induced DSBs, knocking-down A3G expression in cells should result in defective DSB repair. A3G knockdown or control H9 cells were generated by expression of specific A3G-directed shRNA (H9-shA3G) or control shRNA (H9-shCtr1). As shown by FIG. 8F, A3G expression in H9-shA3G cells was reduced by approximately 70-80 percent compared to H9-shCtr1 cells, and was comparable to non-stimulated human primary peripheral blood mononuclear cells (PBMCs). A3G levels in H9-shCtr1 cells resembled those in activated PBMCs stimulated with phytohemagglutinin (PHA). To determine whether A3G is required for DSB repair, the inventors compared the dynamics of γ-H2AX dephosphorylation in IR-exposed H9-shCtr1 versus H9-shA3G cells. FIG. 8G shows that γ-H2AX foci formation in the first hour following IR was similar in both H9-shCtr1 and H9-shA3G cells, and resembled the parental H9 cells. At 8 hours after IR, most H9-shCtr1 cells were negative for γ-H2AX staining and several cells contained minor γ-H2AX foci, reflecting efficient DSB repair. However, γ-H2AX foci in H9-shA3G cells did not decrease in the following hours. Instead, these cells had increased γ-H2AX foci formation over 8 hours, encompassing the vast majority of cells and signifying extensive DNA damage. FIG. 8G (insets) illustrates occasional nuclear fragmentation evident in these cells as shown by DAPI staining.

To test whether A3G indirectly reduces the cellular DSB load by inhibiting apoptosis, cells were preincubated with the caspase inhibitor z-VAD-fmk and probed for γ-H2AX 6 hours after IR. DSB incidence in the presence of z-VAD-fmk was comparable with cells treated with mock, suggesting that A3G has a direct role in DSB repair (FIG. 8H). Depletion of A30 in AIH-77 multiple myeloma cells also resulted in higher DSB incidence 6 hours after IR, indicating that A30 activity is not restricted to H9 cells (FIG. 81). Similar to H9 cells, the elevated DSB load in ARI-77-shA3G cells was caspase-independent.

ATR associates with the regulatory protein ATRIP which has been proposed to localize ATR to sites of DNA damage through an interaction with single-stranded DNA (ssDNA) coated with replication protein A (RPA). The disruption in DSB repair exhibited in H9-shA3G cells following IR may interfere with RPA-ATR mediated response, and as a consequence, with cell cycle checkpoints. Cell-cycle checkpoint activation was analyzed by measuring the DNA content of H9, H9-shCtr1 and H9-shA3G cells 20 hours following 4 Gy IR. FIG. 8J demonstrates that both H9 and H9-shCtr1 cells were arrested in the G2M phase, accounting for a comparable decrease in G0/G1 and S-phase cells. In sharp contrast, H9-shA3G cells did not undergo G2M arrest but instead had an increased sub-G1 fraction, which might signify nuclear fragmentation occurring during mitosis of damaged cells or cell death in the absence of A3G. To conclude, the inventors surmise that A3G is required for efficient DSB repair and for establishing the RPA-ATR mediated response following IR.

### Example 8

A3G-Mediated DSB Repair is Cytidine Deaminase Dependent

To assess whether A3G cytidine deaminase activity is required for DSB repair, the inventors examined whether IR-Induced ₇412AX focus formation occurred differentially in SupTH cells stably expressing A3G, an E259Q catalytic mutant (as denoted by SEQ ID NO. 90) or empty vector (EV). As shown in FIG. 9A, the SupT11 cell line is a single-cell subclone of SupT1 that is nearly devoid of all endogenous APOBEC3s, including A3G. The average DSB load as assessed by γ-H2AX focus formation was approximately 2-fold lower in wild-type A3G-expressing cells compared with the EV control cells 24 hours after IR, demonstrating that reconstitution of A3G expression in these cells enhances DSB repair (FIG. 9B). In contrast, enhanced repair was not observed in A3G E259Q-expressing cells.

### Example 9

Cytidine Deamination-Dependent Recruitment of RPA to ssDNA

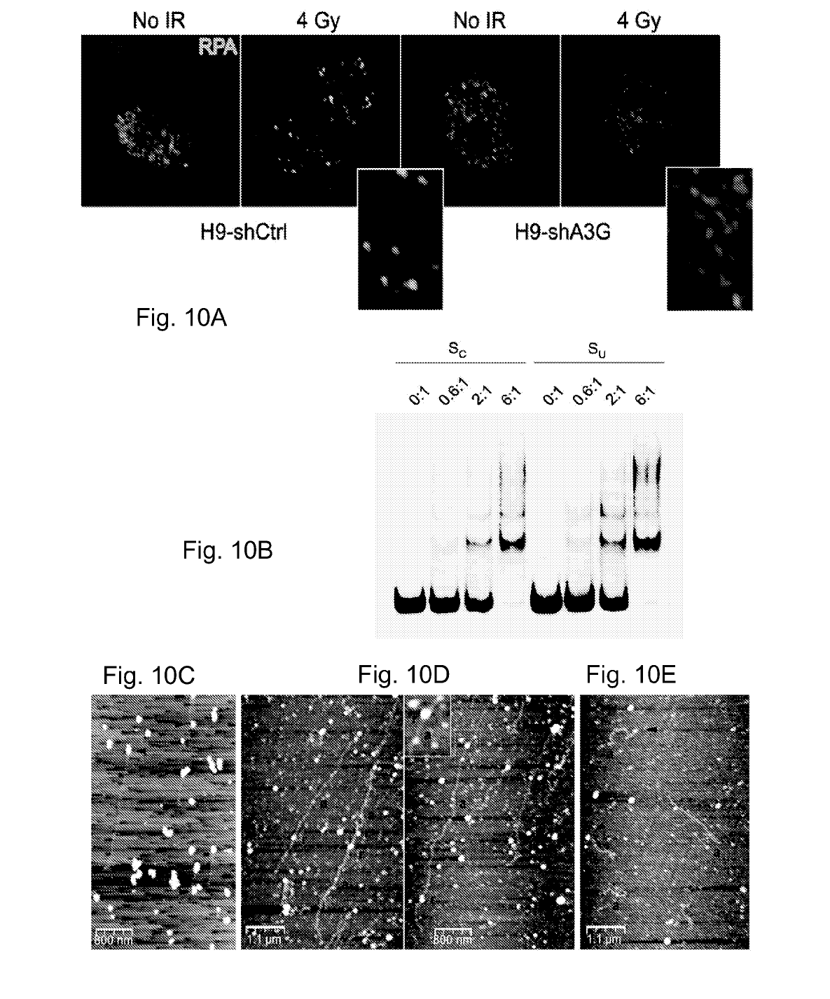

RPA is required for the recruitment of ATR to sites of DNA damage and for ATR-mediated Chk1 activation and G2M arrest. RPA32 subunit interacts with the human uracil-DNA glycosylase UNG2, and RPA co-localizes with UNG2 and deoxyuridine (dU) in replication foci. The delayed defect in DSB repair in the absence of A3G shown in FIGS. 8G and 8J could imply that A3G affects the RPA-ATR cascade. RPA and ATR are recruited to damage-induced subchromatin microcompartments delineated by ssDNA. Therefore, the inventors determined whether A3G is required for RPA foci formation following IR. FIG. 10A demonstrates a diffused nuclear localization of RPA in non-irradiated H9-shCtr1 and H9-shA3G cells. Whereas RPA was recruited to damage-induced foci in irradiated H9-shCtr1, it did not form such foci in H9-shA3G cells following IR. Interestingly, A3G did not co-localize with RPA, but instead was adjacent or in close proximity to RPA (FIG. 10A, inset), suggesting that A3G modulates RPA localization indirectly rather than by direct interaction.

The inventors have previously shown that the catalytically active form of A3G is monomeric [8]. The disassembly of A3G multimers observed in the presence of ssDNA therefore activates A3G catalysis, which might directly recruit RPA to dU-containing ssDNA. Binding of purified RPA to oligonucleotides (80 nt) containing a single dU (S_{U}) or dC (S_{C}) was determined by EMSA. FIG. 10B shows that RPA binding to S_{U} was approximately twofold more efficient than to S_{C}, indicating that RPA recognizes a single dU in ssDNA with enhanced affinity. These results suggest that ssDNA termini induce the disassembly of A3G multimers into the catalytically active monomeric form, which may deaminate the ssDNA cytidines and enhances RPA binding.

DSB resection in-vivo might generate multiple potential targets for A3G-induced cytidine deamination. To simulate RPA recruitment to resected ssDNA, the Rolling Circle Amplification (RCA) system was used to generate ssDNA molecules containing an optimal CCC A3G target site at 100 nt intervals (L_{C}), or control ssDNA containing AAA instead (L_{A}), and probed RPA interactions using AFM. Following 30 min incubation in ice, RPA associated similarly with both substrates. FIG. 10C shows that interaction occurred mainly at one or both ends of the ssDNA, with no additional internal binding, even at RPA:DNA molar excess of 6:1. Occasionally, RPA bound a single internal site inducing a V-like structure in the ssDNA. To determine whether A3G-mediated cytidine deamination recruits RPA to ssDNA, A3G was incubated with L_{C} or L_{A} for 30 min and then RPA was added for further 30 min As shown in FIG. 10D, RPA heterotrimers bound the L_{C} DNA at multiple internal sites, concomitant with A3G monomers. In marked contrast, FIG. 10E shows that RPA association with the L_{A} DNA was mainly at the DNA end, similar to the interaction observed in the absence of A3G. Furthermore, whereas the average length of DNA produced in the inventors system was 1 μm (˜3000 nt), and was not affected by RPA (FIG. 10C) or following incubation of L_{A}+A3G+RPA (FIG. 10E) or L_{C}+A3G (FIG. 10E), the average DNA length measured following incubation of L_{C}+A3G+RPA was 3.4 μm (FIG. 10D), which can be attributed to end-synapsis of several DNA molecules. Hence, cytidine deamination recruits RPA to internal domains in ssDNA, which, in turn, promotes end-synapsis by monomeric A3G. This suggests the role of A3G in DSB repair from DNA resection to completion of HR or NHEJ.

### Example 10

A3G Mediates Deletional Repair of a Persistent IScel-Induced DSB

Deamination of cytidines in resected1DSI3 ssDNA overhangs may result in cleavage of ssDNA in abasic sites, mediated by the base excision repair mechanism or other repair factors. To test whether A3G deaminates resected ssDNA, the inventors expressed A3G or the A3G W285A catalytic mutant (SEQ ID NO. 88 and 81, respectively, which retains wild-type DNA binding properties (see FIG. 3B) in U2OS cells stably carrying a DR-GFP HR reporter cassette [11] and expressing an inducible ISceI-Cherry endonuclease (HRind cells) [10].

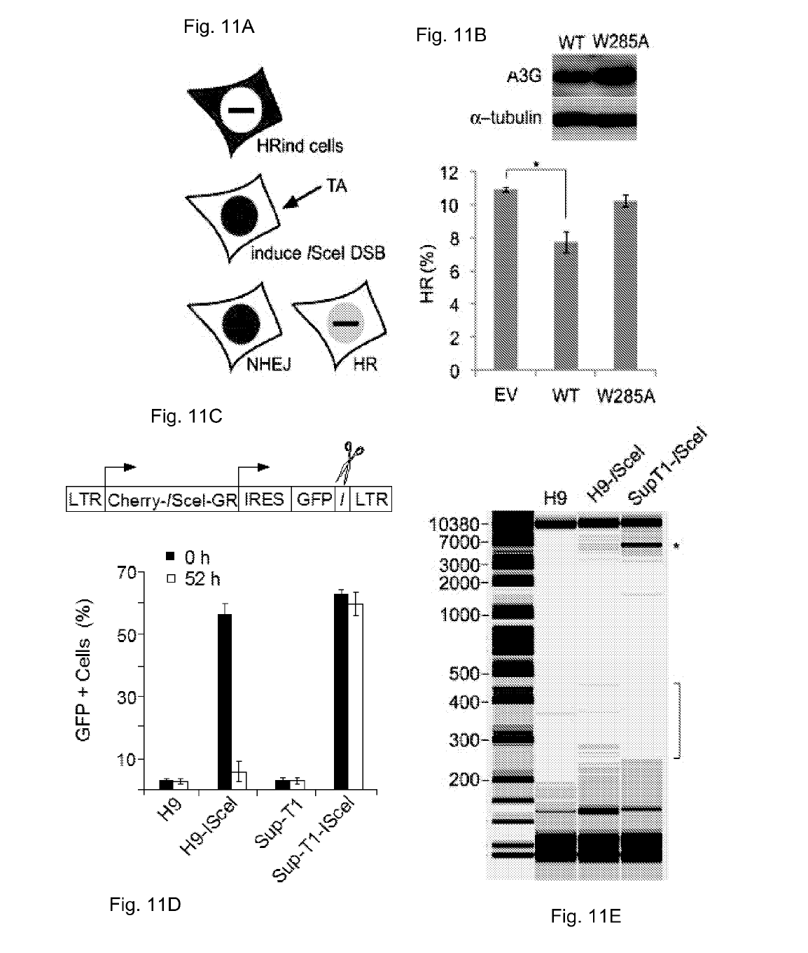

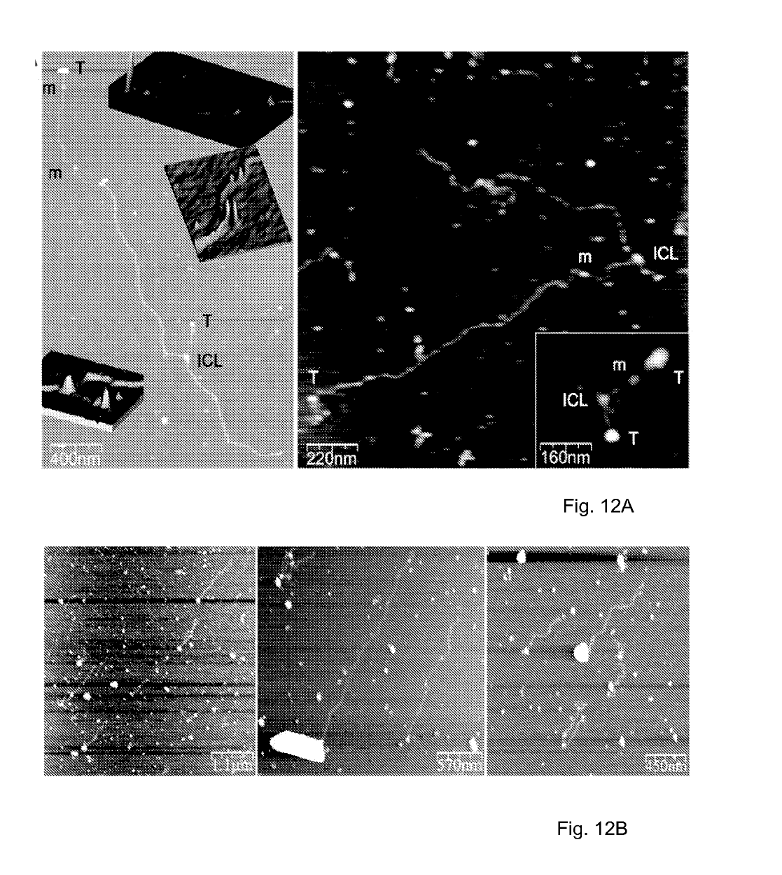

Western blot analysis confirmed that these cells do not express endogenous A3G (results not shown). Induction of ISceI generates a unique DSB within the DR-GFP cassette that produces functional GFP only if repaired by HR (FIG. 11A). The inventors assumed that cytidine deamination in resected ssDNA flanking the ISceI restriction site may lead to truncation of the DR-GFP DNA, resulting in reduced GFP reconstitution. Whereas 10.9% plus or minus 1.0% of the cells underwent repair by HR 52 hours after ISceI induction, expression of wild-type A30 reduced. HR by 30.2% plus or minus 5.6%. In contrast, expression of A3G W285A resulted in only 6.2% plus or minus 3.3% reduction in HR (FIG. 11B). This may indicate that A3G targets resected ssDNA. flanking IScel-induced DSB. To further test the activity of endogenous A3G, a Cherry-ISceI-GR gene was cloned into a lentiviral expression vector and inserted an ISceI target site directly downstream to an IRES-GFP gene (FIG. 11C). After transduction of cells with the lentiviral vector, induction of the ISceI enzyme leads to a unique DSB directly downstream to the GFP gene. In case A3G targeted ssDNA overhangs flanking the DSB after resection, the inventors expected that C to U mutations andor deletion of the GFP coding sequence would result in inactivation of the GFP gene. H9 (A3Ci-high) and SupT1 (A3G-low) cells were infected with lentiviruses containing the ISceI vector (H9-ISceI and SupT1-ISceI cells) or mock, and analyzed for GFP⁺ cells by FACS before and after induction of Cherry-ISceI-GR with TA. Assessment of GFP⁺ cells 48 hours after lentiviral infection indicated that transduction efficiency was 54% to 64% (FIG. 11D). Whereas the percentage of GFP⁺ SupT1-ISceI cells was only marginally reduced 52 hours after ISceI induction, the percentage of GFP⁺ H9-ISceI cells was reduced 9-fold. Notably, microscopic examination of the cells 7 days after ISceI induction revealed poor survival of SupT1-ISceI cells but high viability of H9-ISceI cells (not shown). To test whether the reduction in GFP⁺ H9-ISceI cells is attributed to deletion of the GFP coding sequence, total genomic DNA was extracted from H9-ISceI and SupT1-ISceI cells 52 hours after ISceI induction, and lentiector-specific sequence flanking the ISceI target site was amplified by PCR (FIG. 11E). The 5900-bp DNA band corresponding to the parental lentiviral cassette was markedly reduced in H9-ISceI compared with SupT1-ISceI cells. In addition, several PCR products in the range of 250 to 450 bp were detected after amplification of H9-ISceI DNA but not SupT1-ISceI or H9-mock DNA, suggesting that these PCR products represent truncated DNA junctions. Thus, repair of a persistent ISceI-induced DSB in H9 cells involves mutagenic deaminase-dependent processing of genomic sequences flanking the break.

### Example 11

A3G Mediates Non-Covalent ssDNA Interstrand Crosslinking

In cells, DSBs undergo active resection by cellular nucleases, forming ssDNA overhangs. DSB resection is estimated to generate an average of 2-4 kb long ssDNA in each side of the break. To simulate A3G association with resected ssDNA, the inventors used in-vitro rolling circle amplification to generate ˜1.5-7 (average=3) kb long ssDNA molecules containing an optimal CCC A3G target site at 100 b intervals. Single molecule interactions of purified A3G with ssDNA were probed by atomic force microscopy (AFM). As shown by FIG. 12A, A3G multimers associated predominantly with the ssDNA terminus, whereas A3G monomers occupied internal ssDNA domains. Strikingly, association of A3G multimers with ssDNA termini promoted formation of non-covalent ssDNA interstrand crosslinks (ICLs). To assess the involvement of A3G cytidine deaminase activity in ICL formation, the inventors used the A3G W285A mutant, described by Example 3 and FIG. 3, which binds ssDNA with similar affinity as wild-type A3G but is catalytically dead. FIG. 12B shows that although A3G W285A readily bound the ssDNA termini, it did not induce ICLs, suggesting that cytidine deamination facilitates A3G-induced ICLs. Hence, A3G may promote DSB repair in lymphoma cells by forming transient protein-mediated ssDNA ICLs following DSB resection.

Without being bound by the theory, A3G promotes genomic instability both by direct deamination of dC residues in resected ssDNA, and by transiently forming ICLs. Targeting of dU in resected ssDNA by uracil-DNA glycosylase (UNG) may lead to loss of genetic material following processing by the base excision repair complex. Alternatively, hyper-mutated ssDNA serving as a template for homologous recombination may lead to fixation of G>A mutations in the extended DNA strand, and consequently C>T mutations in the template strand following mismatch repair or DNA replication. Formation of random ICLs may direct non-templated end joining, and therefore give rise to mutations, deletions or chromosomal translocations. Such mutational-biased repair may underlie the predominance of the C>T G>A base substitutions in human cancer, or may be associated with a subset of chromosomal translocations observed in lymphomas.

### Example 12

Native HIV-1-Associated A3G has Reduced Specific Activity

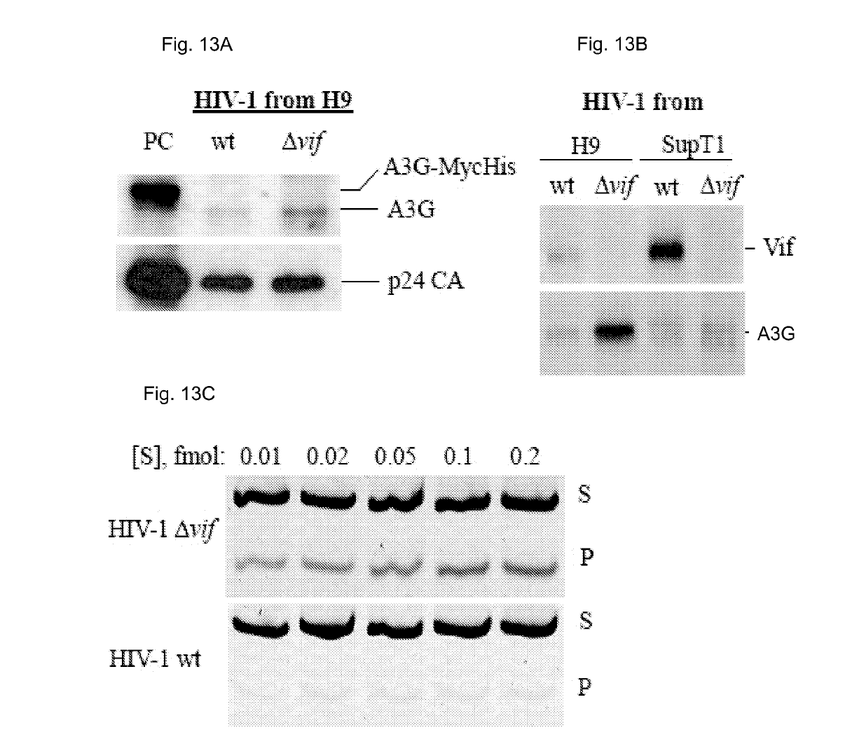

The surprising involvement of A3G in cellular DSB repair prompted the inventors to search for compounds that modulate, and specifically inhibit, its activity or expression. Since the viral infectivity factor (Vif) is known as promoting the degradation of A3G, the inventors next explored the relationship between Vif and A3G. It has already been shown that the ssDNA deamination activity of A3G can severely damage viral DNA. The currently known viral mechanism coping with this threat is the targeting of A3G to proteasomal degradation by the viral Vif protein. In the absence of Vif, A3G deaminates dC residues in the viral negative-strand DNA synthesized by reverse transcription post-entry to the target cell. As Vif is packaged in the virion, the inventors were interested to reveal whether it directly inhibits A3G enzymatic activity. FIG. 13A shows that wt virions released from H9 cells contain reduced amounts of A3G molecules compared to that associated with Δvif particles. Quantification of the bands revealed that the wt particles encapsidated 5.8 times lower (approx. 17%) A3G protein than in Δvif particles (see Table 1 below). In HIV-1 infected H9 cells, Vif counteracts the cellular A3G mainly by targeting it for proteosomal degradation, reducing the intracellular amounts of both proteins, leading to decreased incorporation of A3G and Vif into the newly assembled wt particles. FIG. 13B shows that wt particles released from H9 cells contain reduced amounts of Vif compared to particles released from the permissive Sup T1 cells, which do not express A3G.

TABLE 1
A3G protein content in HIV-1 wt vs. HIV-1 Δvif virions

| | | p24 | | A3G(OD)/ | A3G in HIV-1wt |
|---|---|---|---|---|---|
| | | ng/ | A3G/slot, | ng of | vs HIV-1Δvif, |
| Virus | Exp. | slot | (OD) | p24 | per ng p24, (%) |
| HIV-1 wt | #1 | 58.58 | 98592 | 1683 | 21.60 |
| HIV-1 Δvif | | 33.68 | 262441 | 7792 | |
| HIV-1 wt | #2 | 58.58 | 27949 | 477 | 15.71 |
| HIV-1 Δvif | | 33.68 | 102255 | 3036 | |
| HIV-1 wt | #3 | 29.29 | 25501 | 871 | 15.04 |
| HIV-1 Δvif | | 16.84 | 97458 | 5787 | |
| HIV-1 wt | #4 | 29.29 | 6667 | 228 | 17.98 |
| HIV-1 Δvif | | 16.84 | 21321 | 1266 | |
| A3Gwt vs | | | | | 17.58 ± 2.96 |
| A3GΔvif | | | | | |

The deaminase activity associated with the wt and Vif deficient viruses released from H9 cells correlates to the amounts of A3G molecules entrapped in the particles (FIG. 13C). However, calculation of the efficacy of the A3G enzymes packed in the wt and Δvif particles revealed that the deaminase activity of A3G in the wt particle is significantly lower than expected (Table 2) and is only 6.42% of the deaminase activity in Δvif particles. Normalizing the virion-associated deaminase activity to A3G protein content indicates that the specific activity of wt HIV-1-associated A3G enzyme is 36.5% of the enzyme associated with the Δvif particles (6.4217.58*100; see Tables 1 and 2). A plausible explanation of these results is that the Vif molecules associated with the HIV-1 wt particles inhibit the intrinsic deamination activity of A3G.

TABLE 2
Deaminase activity (DA) of A3G in HIV-1wt vs HIV-1Δvif virions

| | | Deaminase | |
|---|---|---|---|
| | [S], fmol/ | activity (DA), | DA in HIV-1wt vs |
| | reaction | [P]/([P] + [S]) | HIV-1 Δvif - % |
| HIV-1 wt | 0.02 | 0.0135 | 5.81 |
| HIV-1 Δvif | | 0.2320 | |
| HIV-1 wt | 0.05 | 0.0185 | 6.79 |
| HIV-1 Δvif | | 0.2727 | |
| HIV-1 wt | 0.2 | 0.0194 | 5.99 |
| HIV-1 Δvif | | 0.3243 | |
| HIV-1 wt | 0.5 | 0.0228 | 7.08 |
| HIV-1 Δvif | | 0.3224 | |
| A3G wt vs | | | 6.42 ± 0.62 |
| A3G Δvif | | | |

Each reaction was loaded with equal amounts (1.25 ng of p24) of HIV-1 wt or HIV-1 Δvif purified virus.

### Example 13

Inhibition of A3G Deaminase Activity by HIV-I Vif

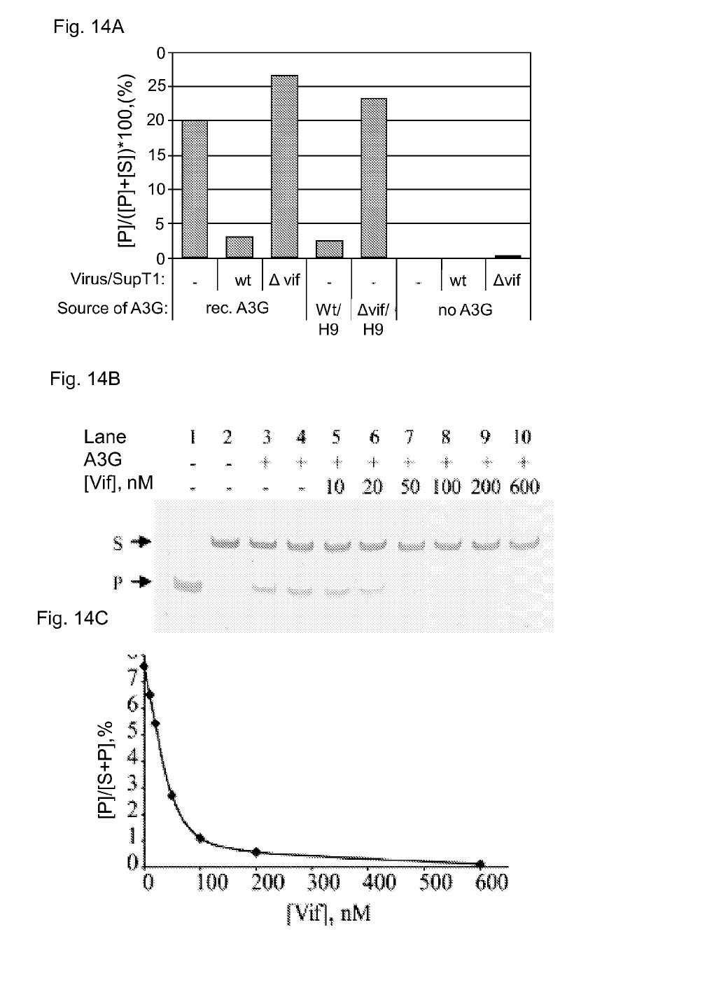

To determine whether Vif molecules associated with HIV-1 particles inhibit A3G activity, the inventors performed exogenous reactions using purified recombinant A3G. A3G alone deaminated 20% of the dC in the oligonucleotide substrate. Adding particles released from wt HIV-1 infected Sup T1 cells, which contain Vif (see FIG. 13B) decreased A3G activity, while adding the same amount of Δvif particles to the reaction had no effect, or even slightly amplified the deaminase activity, probably because of increased concentration of proteins in the mixture (FIG. 14A). These results indicate that Vif molecules associated with HIV-1 are able to inhibit A3G activity in vitro.

Next, an in-depth biochemical analysis of Vif-mediated inhibition of A3G enzymatic activity was preformed. To this end the inventors expressed and purified recombinant Vif protein and examined A3G activity in the presence of Vif. The purification of recombinant His-tagged Vif from bacteria yielded an over 90% purified protein, as revealed by SDS-PAGE analysis and verified by Western blotting. Purified A3G was incubated with increasing amounts of purified Vif and the effect on A3G-mediated deamination levels of the ss-deoxyoligonucleotide substrate was measured. FIG. 14B clearly shows that Vif inhibits A3G deaminase activity in a dose-dependent manner (lanes 5-10). An equal amount of an elution fraction from non Vif-expressing bacteria (lane 4) or 10 μM BSA (lane 3) used as controls did not inhibit A3G activity. The inhibitory effect exerted by Vif was observed at Vif concentrations ranging down to 10 nM. FIG. 14C is a graphic presentation of the results shown in FIG. 14B.

### Example 14

Identifying the Vif-Derived Peptides Which Inhibit A3G Deaminase

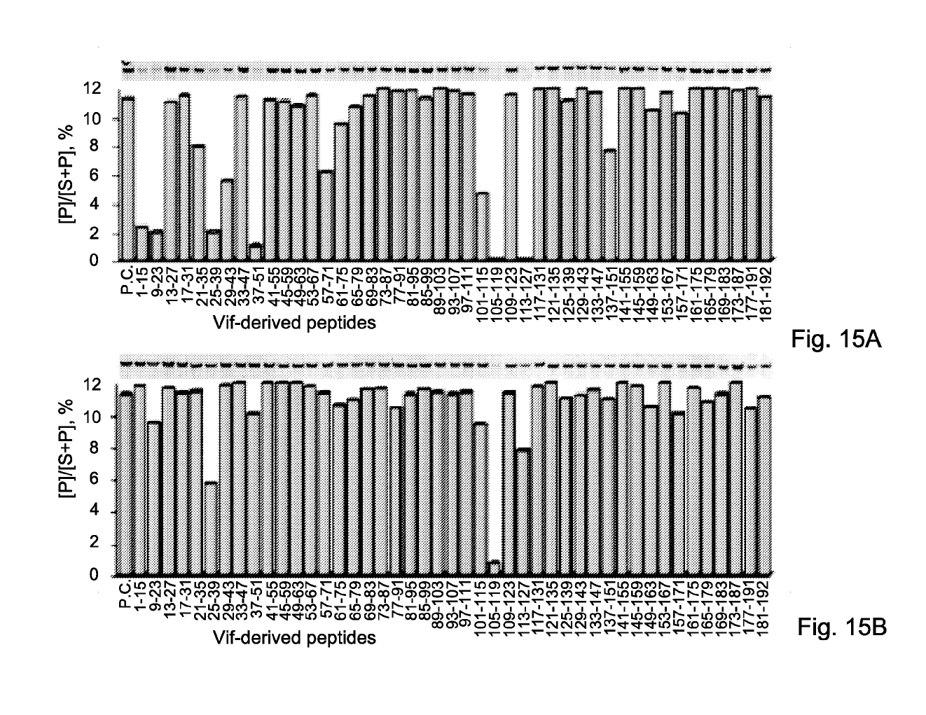

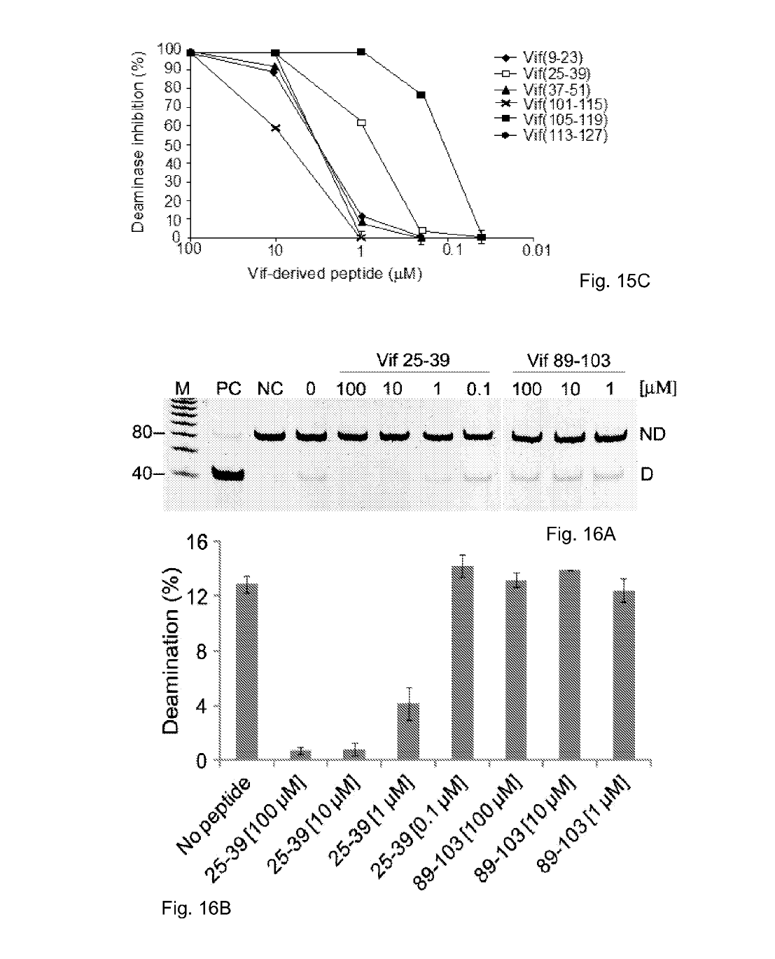

In order to identify the inhibitory domains in Vif, a battery of 15-mer 46 Vif-derived peptides (Table 3) with 11aa overlaps covering the full-length protein was screened for the inhibition of A3G-mediated deamination. Peptides corresponding to sequences throughout the Vif protein exerted a vast inhibitory effect at a concentration of 100 μM. As shown by FIG. 15A, at a lower concentration of 10 μM, a distinct inhibitory pattern was observed by peptide sequences corresponding to Vif N-terminus (1-51aa; also denoted as SEQ ID NO. 51) and to a central region (101-127aa; also denoted as SEQ ID NO. 52), previously characterized as a novel zinc-binding domain. Specifically, six peptides inhibited the A3G deaminase activity at a lower concentration of 1 μM, mapping the inhibitory sequences to Vif9-23 (SEQ ID NO.:3), Vif25-39 (SEQ ID NO.:7) and Vif37-51 (SEQ ID NO.:10) at the N-terminal region, and Vif101-115 (SEQ ID NO.:26), Vif105-119 (SEQ ID NO.:27 and Vif113-127 (SEQ ID NO.:29) at the central region, as seen in FIG. 15B. These peptides were further analyzed for the inhibition of A3G activity at lower concentrations ranging down to 40 nM. As illustrated by FIG. 15C, the peptides Vif25-39 (SEQ ID NO.:7) and Vif105-119 (SEQ ID NO.:27), significantly reduced the A3G activity at 1 μM and 0.2 μM with an IC₅₀ of approximately 0.6 μM and 0.1 μM, respectively. To exclude the possibility that the Vif-derived peptides decrease overall PCR efficiency, these experiments were verified by real-time PCR assays that showed no differential amplification rate of A3G reaction products with or without the Vif-derived peptides. FIGS. 16A and 16B present an experimental repeat focusing on the inhibition of A3G deaminase activity by Vif25-39 as compared to the control peptide Vif89-103. The Vif25-39 peptide specifically inhibited the deaminase activity of purified A3G with an IC₅₀ of approximately 1 μM, unlike Vif89-103 which did not inhibit A3G at a concentration of 100 μM and used as a control peptide (FIG. 16A).

TABLE 3
Vif-derived peptides

| SEQ | | | |
|---|---|---|---|
| ID | From | To | Peptide |
| NO | position | position | Sequence |
| 1 | 1 | 15 | MENRWQVMIVWQVDR |
| 2 | 5 | 19 | WQVMIVWQVDRMRIR |
| 3 | 9 | 23 | IVWQVDRMRIRTWKS |
| 4 | 13 | 27 | VDRMRIRTWKSLVKH |
| 5 | 17 | 31 | RIRTWKSLVKHHMYI |
| 6 | 21 | 35 | WKSLVKHHMYISGKA |
| 7 | 25 | 39 | VKHHMYISGKAKGWF |
| 8 | 29 | 43 | MYISGKAKGWFYRHH |
| 9 | 33 | 47 | GKAKGWFYRHHYEST |
| 10 | 37 | 51 | GWFYRHHYESTHPRI |
| 11 | 41 | 55 | RHHYESTHPRISSEV |
| 12 | 45 | 59 | ESTHPRISSEVHIPL |
| 13 | 49 | 63 | PRISSEVHIPLGDAR |
| 14 | 53 | 67 | SEVHIPLGDARLVIT |
| 15 | 57 | 71 | IPLGDARLVITTYWG |
| 16 | 61 | 75 | DARLVITTYWGLHTG |
| 17 | 65 | 79 | VITTYWGLHTGERDW |
| 18 | 69 | 83 | YWGLHTGERDWHLGQ |
| 19 | 73 | 87 | HTGERDWHLGQGVSI |
| 20 | 77 | 91 | RDWHLGQGVSIEWRK |
| 21 | 81 | 95 | LGQGVSIEWRKKRYS |
| 22 | 85 | 99 | VSIEWRKKRYSTQVD |
| 23 | 89 | 103 | WRKKRYSTQVDPDLA |
| 24 | 93 | 107 | RYSTQVDPDLADQLI |
| 25 | 97 | 111 | QVDPDLADQLIHLYY |
| 26 | 101 | 115 | DLADQLIHLYYFDCF |
| 27 | 105 | 119 | QLIHLYYFDCFSESA |
| 28 | 109 | 123 | LYYFDCFSESAIRNA |
| 29 | 113 | 127 | DCFSESAIRNAILGH |
| 30 | 117 | 131 | ESAIRNAILGHIVSP |
| 31 | 121 | 135 | RNAILGHIVSPRCEY |
| 32 | 125 | 139 | LGHIVSPRCEYQAGH |
| 33 | 129 | 143 | VSPRCEYQAGHNKVG |
| 34 | 133 | 147 | CEYQAGHNKVGSLQY |
| 35 | 137 | 151 | AGHNKVGSLQYLALA |
| 36 | 141 | 155 | KVGSLQYLALAALIT |
| 37 | 145 | 159 | LQYLALAALITPKKI |
| 38 | 149 | 163 | ALAALITPKKIKPPL |
| 39 | 153 | 167 | LITPKKIKPPLPSVT |
| 40 | 157 | 171 | KKIKPPLPSVTKLTE |
| 41 | 161 | 175 | PPLPSVTKLTEDRWN |
| 42 | 165 | 179 | SVTKLTEDRWNKPQK |
| 43 | 169 | 183 | LTEDRWNKPQKTKGH |
| 44 | 173 | 187 | RWNKPQKTKGHRGSH |
| 45 | 177 | 191 | PQKTKGHRGSHTMNG |
| 46 | 181 | 192 | KGHRGSHTMNGH |

*Fifteen-mer peptides derived from HIV-1 Vif (sequence accession: #AAZ14773) covering the full-length protein with 11aa overlaps.

### Example 15

### Determining the Mode of Vif-Mediated Inhibition of A3G Enzyme

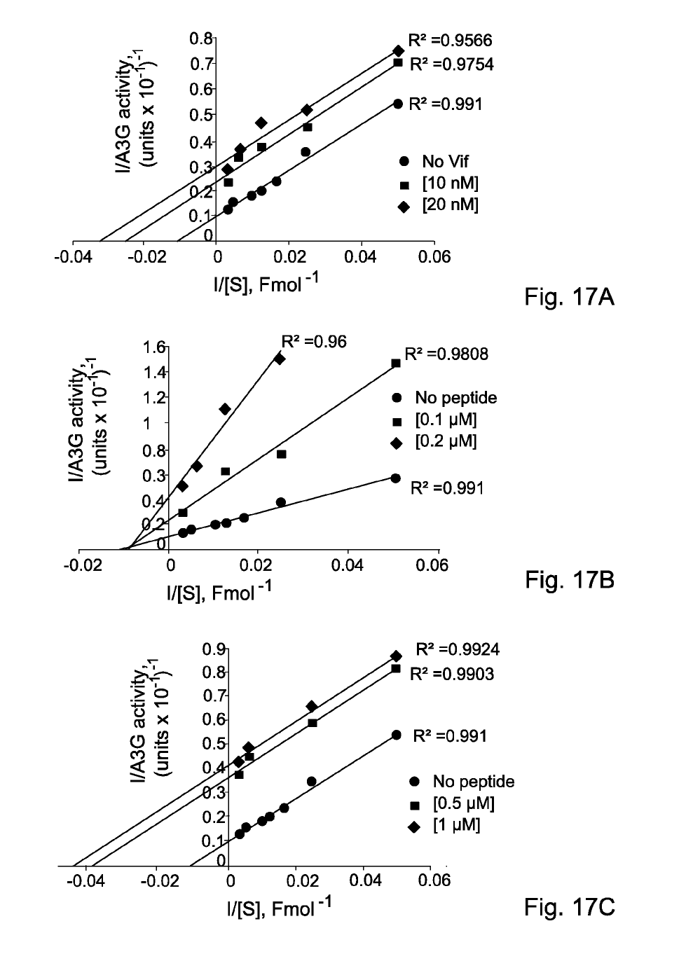

The inventors determined the mode of inhibition employed by Vif and the Vif-derived peptides corresponding to residues 25-39 (SEQ ID NO.:7) and 105-119 (SEQ ID NO.:27). A3G initial deamination rates were measured in the presence of 10 and 20 nM Vif (FIG. 17A), 0.1 and 0.2 μM Vif105-119 (SEQ ID NO.:27) (FIG. 17B) or 0.5 and 1 μM Vif25-39 (SEQ ID NO.:7) (FIG. 17C). The double-reciprocal plot of A3G inhibition by Vif and Vif25-39 reveals an uncompetitive inhibition mode, whereas Vif105-119 inhibits A3G in a mixed mode. Accordingly, the K′I values of Vif and Vif25-39 are approximately 8.7*10⁻⁹ M and 2.5*10⁻⁷ M, respectively. The K′I and KI values of Vif105-119 are approximately 9.2*10⁻⁸ M and 6.8*10⁻⁸ M, respectively.

### Example 16

Vif25-39 Inhibits DSB Repair in Cultured Cells

Having established the mode of action of Vif25-39 in-vitro, the inventors proceeded to determine the effects Vif25-39 performs ex-vivo, in cultured cells. H9 cells were incubated for 2 hours with Vif25-39 (SEC ID NO. 7), Vif89-103 (SEQ ID NO. 23, control) or non peptide, irradiated (4 Gy) or mock-irradiated (No IR) and stained following 8 h with anti-A3G, anti-γ-H2AX antibodies. Nuclei were counter-stained with DAPI. Cells pre-incubated with Vif89-103 exhibited efficient DSB repair (<8% of cells containing DSBs), similar to cells incubated with mock or non-irradiated cells pre-incubated with Vif25-39 (FIGS. 18A and 18B). In contrast, 32±4.3% of irradiated cells pre-incubated with Vif25-39 contained DSBs, suggesting that cytidine deamination is required for A3G-mediated DSB repair Importantly, Vif25-39 did not prevent recruitment of A3G to DSBs, as A3G colocalized with γ-H2AX nuclear foci at unrepaired DSBs in pre-treated cells (FIG. 18C).

### Example 17

Identifying Consensus Sequences for Vif and A3F-Derived Peptides Which Inhibit A3G Deaminase Activity

As shown above, the inventors now identified two main domains on Vif, specifically, Vif25-39 (SEQ ID NO.:7) and Vif105-119 (SEQ ID NO.:27), that significantly reduced the A3G activity. In order to identify more efficient inhibitors and to re-define the inhibitory domains and consensus sequences in Vif, a battery of short Vif-derived peptides (Table 4) was screened for the inhibition of A3G-mediated deamination. The inventors further examined peptides derived from A3F that may also contain similar inhibitory sequences.

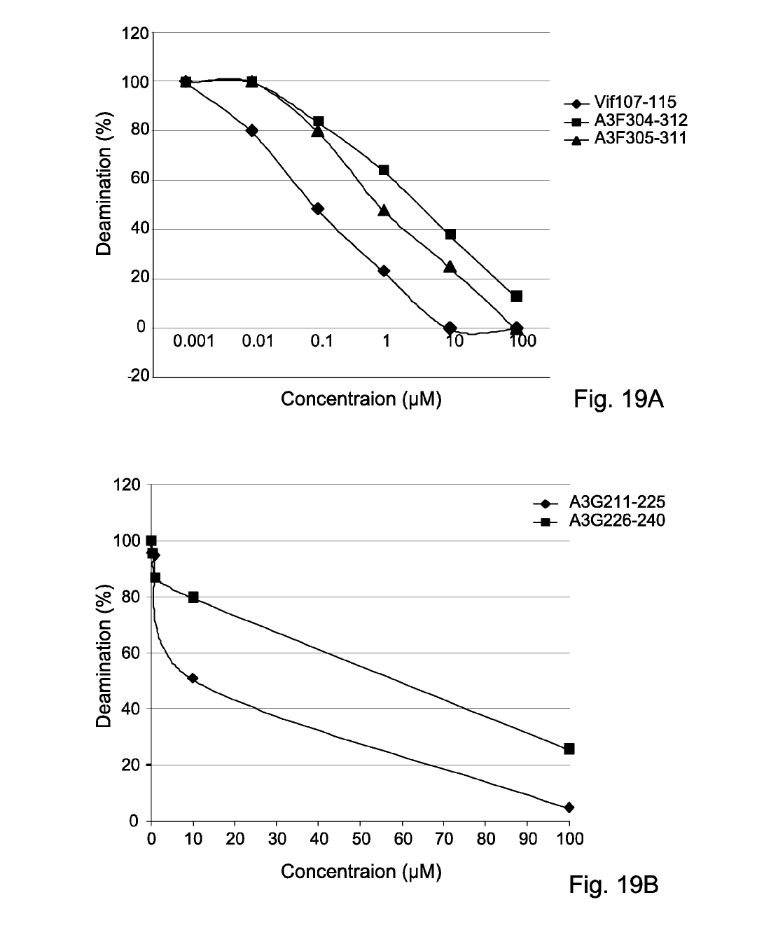

As shown by FIG. 19A, at a lower concentration of 0.001 to 10 μM, a distinct inhibitory pattern was observed by peptide sequences corresponding to Vif residues 107-115 (also denoted as SEQ ID NO. 71). This sequence corresponds to a central region previously characterized as a novel zinc-binding domain. Two other peptides derived from A3F, comprising the consensus sequence X-Leu-Tyr-Tyr-Phe (as denoted by SEQ ID NO. 67), specifically the peptides of SEQ ID NO. 74 and 75, showed similar inhibitory effect.

The inventors next examined the potential inhibitory action of A3G-derived peptides. As shown in FIG. 19B, two A3G peptides comprising residues 211-225 or 226-240 (SEQ ID NO. 83, 84, respectively) showed a marked inhibitory effect.

These results demonstrate the feasibility of using inhibitory peptides for inhibiting DSB repair processes mediated by A3G.

TABLE 4
shortest Vif-, A3F and A3F-derived peptides

| SEQ | | | | |
|---|---|---|---|---|
| ID | | From | To | Peptide |
| NO | Origin | position | position | Sequence |
| 66 | Vif or A3F | | | VKHH |
| 67 | Vif or A3F | | | XLYYF |
| 68 | Vif or A3F | | | KGWF |
| 69 | Vif | 23 | 30 | SLVKHHMY |
| 70 | A3F | 224 | 231 | VVKHHSPV |
| 71 | Vif | 107 | 115 | IHLYYFDCF |
| 72 | Vif | 108 | 113 | HLYYFD |
| 73 | Vif | 108 | 112 | HLYYF |
| 74 | A3F | 304 | 312 | ARLYYFWDT |
| 75 | A3F | 305 | 311 | RLYYFWD |
| 76 | Vif | 30 | 39 | YISGKAKGWF |
| 77 | Vif | 25 | 34 | VKHHMYISGK |
| 78 | Vif | 33 | 39 | GKAKGWF |
| 79 | Vif | 109 | 115 | LYYFDCF |
| 80 | Vif | 109 | 117 | LYYFDCFSE |
| 81 | Vif | 36 | 39 | KGWF |
| 82 | Vif | 109 | 112 | LYYF |
| 83 | A3G | 211 | 225 | WVRGRHETYLCYEVE |
| 84 | A3G | 226 | 240 | RMHNDTWVLLNQRRG |
| 85 | A3G | 226 | 231 | RMHNDT |
| 86 | A3G | 211 | 240 | WVRGRHETYLCYEVE |
| | | | | RMHNDTWVLLNQRRG |
| 87 | Vif | 118 | 127 | SAIRNAILGH |

1. A method of modulating double stranded DNA breaks (DSB) repair processes in a subject in need thereof, comprising the step of administering to said subject a therapeutically effective amount of at least one compound that modulates the expression or activity of at least one Apolipoprotein B mRNA-editing enzyme-catalytic polypeptide-like (APOBEC) family member, or of any composition comprising the same, wherein said activity is at least one of cytidine deaminase activity and single stranded DNA (ssDNA) tethering.

2. (canceled)

3. The method according to claim 1, for treating a proliferative disorder in a subject in need thereof by inhibiting DSB repair processes in cells of said subject, said method comprises the step of: administering to said subject a therapeutically effective amount of at least one compound that inhibits the expression or the activity of at least one said APOBEC family member, or of any composition comprising the same, wherein said APOBEC family member is APOBEC3G (A3G).

4. (canceled)

5. The method according to claim 3, wherein said compound that inhibits the cytidine deaminase activity of said APOBEC family member is at least one of a retrovirus viral infectivity factor (Vif) polypeptide, or any functional fragments, peptide, derivative or homologue thereof and a peptide derived from an APOBEC family member or any combination thereof.

6. The method according to claim 5, wherein said compound is an isolated peptide comprising any one of:
a. an amino acid sequence of at least one of Val¹-Lys²-His³-His⁴, as denoted by SEQ ID NO. 66, Lys¹-Gly²-Trp³-Phe⁴ as denoted by SEQ ID NO. 68 and X¹Leu²Tyr³Tyr⁴-Phe⁵ as denoted by SEQ ID NO. 67, wherein X₁ is a positively charged amino acid selected from His and Arg; and wherein said peptide is derived from any one of HIV-1 viral infectivity factor (Vif) and APOBEC3F (A3F); and
b. a peptide derived from residues 211-240 of A3G.

7. (canceled)

8. The method according to claim 6, wherein said peptide comprises an amino acid sequence of any one of residues 25-39, 105-119 and 107-115 of HIV-1 Vif, as denoted by SEQ ID NO. 7, 27 and 71, respectively, residues 304-312 and 305-311 of A3F as denoted by SEQ ID NO. 74 and 75, respectively and residues 211-225 and 226-240 of A3G as denoted by SEQ ID NO. 83 and 84, respectively, or any fragments, derivatives, homologues, or any combination thereof.

9. The method according to claim 3, wherein said compound that inhibits the expression of at least one said APOBEC family member is at least one of:
I. a nucleic acid inhibitor specific for APOBEC, said inhibitor is any one of shRNA, siRNA, ribozyme or antisense RNA, or any functional fragments thereof, any combination thereof, or any vector comprising the same; and
II. a mutated A3G molecule devoid of cytidine deaminase activity, said mutant comprises at least one of W285A and E259Q substitutions.

10. (canceled)

11. The method according to claim 1, for sensitization of a subject suffering from a proliferative disorder to a genotoxic treatment, said method comprises the step of administering to said subject a therapeutically effective amount of at least one compound that inhibits the expression or the activity of at least one said APOBEC family member, or of any composition comprising the same, wherein said compound is any one of:
I. a vif polypeptide or any fragment or peptide thereof, or any peptide derived from an APOBAC family member, wherein said peptide comprises any one of:
a. an amino acid sequence of at least one of Val¹-Lys²-His³-His⁴, as denoted by SEQ ID NO. 66, Lys¹-Gly²-Trp³-Phe⁴ as denoted by SEQ ID NO. 68 and X¹-Leu²-Tyr³-Tyr⁴-Phe⁵ as denoted by SEQ ID NO. 67, wherein X₁ is a positively charged amino acid selected from His and Arg; and wherein said peptide is derived from any one of HIV-1 viral infectivity factor (Vif) and APOBEC3F (A3F); and
b. a peptide derived from residues 211-240 of A3G;
II. at least one nucleic acid inhibitor specific for APOBEC, said inhibitor is any one of shRNA, siRNA, ribozyme or antisense RNA, or any functional fragments thereof, any combination thereof, or any vector comprising the same; and
III. a mutated A3G molecule devoid of cytidine deaminase activity, said mutant comprises at least one of W285A and E259Q substitutions;
wherein said compound is administered before, simultaneously with, after or any combination thereof, with said genotoxic treatment.

12. (canceled)

13. The method according to claim 11, wherein said subject is suffering from a genotoxic-drug resistant proliferative disorder.

14. The method according to claim 1, for treating a disorder associated with DSB damage in a subject in need thereof by enhancing DSB repair processes in said subject, said method comprises the step of: administering to said subject a therapeutically effective amount of at least one compound that increase or induce the expression or deaminase activity of at least one said APOBEC family member, or of any composition comprising the same.

15. An isolated peptide comprising at least one of:
a. an amino acid sequence of at least one of Val¹-Lys²-His³-His⁴, as denoted by SEQ ID NO. 66, Lys¹-Gly²-Trp³-Phe⁴ as denoted by SEQ ID NO. 68 and X¹-Leu²-Tyr³-Tyr⁴-Phe⁵ as denoted by SEQ ID NO. 67, wherein X₁ is a positively charged amino acid selected from His and Arg; and wherein said peptide is derived from any one of HIV-1 viral infectivity factor (Vif) and APOBEC3F (A3F); and
b. a peptide derived from residues 211-240 of A3G.

16. (canceled)

17. The peptide according to claim 15, comprising an amino acid sequence of any one of residues 25-39, 105-119 and 107-115 of HIV-1 Vif, as denoted by SEQ ID NO. 7, 27 and 71, respectively, residues 304-312 and 305-311 of A3F as denoted by SEQ ID NO. 74 and 75, respectively and residues 211-225 and 226-240 of A3G as denoted by SEQ ID NO. 83 and 84, respectively, or any fragments, derivatives, homologues, or any combination thereof.

18. A composition comprising as an active ingredient at least one peptide according to claim 15, said peptide comprises at least one of:
a. an amino acid sequence of at least one of Val¹-Lys²-His³-His⁴, as denoted by SEQ ID NO. 66, Lys¹-Gly²-Trp³-Phe⁴ as denoted by SEQ ID NO. 68 and X¹-Leu²-Tyr³-Tyr⁴-Phe⁵ as denoted by SEQ ID NO. 67, wherein X₁ is a positively charged amino acid selected from His and Arg; and wherein said peptide is derived from any one of HIV-1 viral infectivity factor (Vif) and APOBEC3F (A3F); and
b. a peptide derived from residues 211-240 of A3G.

19. (canceled)

20. The composition according to claim 18, wherein said peptide comprises an amino acid sequence of any one of residues 25-39, 105-119 and 107-115 of HIV-1 Vif, as denoted by SEQ ID NO. 7, 27 and 71, respectively, residues 304-312 and 305-311 of A3F as denoted by SEQ ID NO. 74 and 75, respectively and residues 211-225 and 226-240 of A3G as denoted by SEQ ID NO. 83 and 84, respectively, or any fragments, derivatives, homologues, or any combination thereof.

21. A pharmaceutical composition according to claim 18, for treating a pathological disorder in a subject in need thereof by reducing, inhibiting or attenuating the activity of at least one APOBEC family member, wherein said composition inhibits DBS repair process in said subject thereby sensitizing said cells to a genotoxic treatment, said composition optionally further comprises a pharmaceutically acceptable excipient or carrier.

22. (canceled)

23. A combined composition comprising as an active ingredient a therapeutically effective amount of at least one compound that modulates the expression or activity of at least one APOBEC family member and at least one additional therapeutic agent, wherein said additional therapeutic agent is a genotoxic insult-inducing agent and wherein said additional therapeutic agent is a genotoxic insult-inducing agent and wherein said compound inhibits at least one of the expression or activity of at least one APOBEC family member said compound being any one of:
a vif polypeptide or any fragment or peptide thereof, or any peptide derived from an APOBEC family member said compound being any one of
a vif polypeptide or any fragment or peptide thereof, or any peptide derived from an APOBAC family member according to claim 15, wherein said peptide comprises any one of:
a. an amino acid sequence of at least one of Val¹-Lys²-His³-His⁴, as denoted by SEQ ID NO. 66, Lys¹-Gly²-Trp³-Phe⁴ as denoted by SEQ ID NO. 68 and X¹-Leu²-Tyr³-Tyr⁴-Phe⁵ as denoted by SEQ ID NO. 67, wherein X₁ is a positively charged amino acid selected from His and Arg; and wherein said peptide is derived from any one of HIV-1 viral infectivity factor (Vif) and APOBEC3F (A3F); and
a peptide derived from residues 211-240of A3G;
II. at least one nucleic acid inhibitor specific for APOBEC, said inhibitor is any one of shRNA, siRNA, ribozyme or antisense RNA, or any functional fragments thereof, any combination thereof, or any vector comprising the same; and
III. a mutated A3G molecule devoid of cytidine deaminase activity, said mutant comprises at least one of W285A and E259Q substitutions.

24. (canceled)

25. A kit comprising:
A. at least one compound that inhibits the expression or activity of at least one APOBEC family member, optionally, in a first unit dosage form, wherein said compound is any one of:
I. a vif polypeptide or any fragment or peptide thereof, or any peptide derived from an APOBEC family member according to claim 15, wherein said peptide comprises any one of:
an amino acid sequence of at least one of Val¹-Lys²-His³-His⁴, as denoted by SEQ ID NO. 66, Lys¹-Gly²-Trp³-Phe⁴ as denoted by SEQ ID NO. 68 and X¹-Leu²-Tyr³-Tyr⁴-Phe⁵ as denoted by SEQ ID NO. 67, wherein X₁ is a positively charged amino acid selected from His and Arg; and wherein said peptide is derived from any one of HIV-1 viral infectivity factor (Vif) and APOBEC3F; and
a peptide derived from residues 211-240 or A3G;
II. at least one nucleic acid inhibitor specific for APOBEC, said inhibitor is any one of shRNA, siRNA, ribozyme or antisense RNA, or any functional fragments thereof, any combination thereof, or any vector comprising the same; and
B. at least one genotoxic insult-inducing agent, and a pharmaceutically acceptable carrier or diluent, optionally, in a second unit dosage form.

26. (canceled)

27. of the method according to claim 1, for treating a proliferative disorder in a subject in need thereof, said method comprises the step of: administering to said subject a therapeutically effective amount of at least one isolated peptide comprising at least one of:
a. an amino acid sequence of at least one of Val¹-Lys²-His³-His⁴, as denoted by SEQ ID NO. 66, Lys¹-Gly²-Trp³-Phe⁴ as denoted by SEQ ID NO. 68 and X¹-Leu²-Tyr³-Tyr⁴-Phe⁵ as denoted by SEQ ID NO. 67, wherein X₁ is a positively charged amino acid selected from His and Arg; and wherein said peptide is derived from any one of HIV-1 viral infectivity factor (Vif) and APOBEC3F (A3F); and
b. a peptide derived from residues 211-240 of A3G.

28. The method according to claim 1, for treating a proliferative disorder in a subject in need thereof, wherein said subject is being treated with a genotoxic therapy, said method comprises the step of: administering to said subject a therapeutically effective amount of at least one compound that inhibits the expression or activity of at least one APOBEC family member, said compound is any one of:
I. a vif polypeptide or any fragment or peptide thereof, or any peptide derived from an APOBAC family member, wherein said peptide comprises any one of:
a. an amino acid sequence of at least one of Val¹-Lys²-His³-His⁴, as denoted by SEQ ID NO. 66, Lys¹-Gly²-Trp³-Phe⁴ as denoted by SEQ ID NO. 69 and X¹-Leu²-Tyr³-Tyr⁴-Phe⁵ as denoted by SEQ ID NO. 67, wherein X₁ is a positively charged amino acid selected from His and Arg; and wherein said peptide is derived from any one of HIV-1 viral infectivity factor (Vif) and APOBEC3F (A3F); and
b. a peptide derived from residues 211-240 of A3G;
II. at least one said APOBEC family member is at least one nucleic acid inhibitor specific for APOBEC, said inhibitor is any one of shRNA, siRNA, ribozyme or antisense RNA, or any functional fragments thereof, any combination thereof, or any vector comprising the same; and
III. a mutated A3G molecule devoid of cytidine deaminase activity, said mutant comprises at least one of W285A and E259Q substitutions.

29-31. (canceled)

32. A method for determining the efficacy of a treatment with a genotixic therapy on a subject suffering from a proliferative disorder or for determining a genotoxic treatment regimen for said subject, wherein said genotoxic therapy comprises at least one of chemotherapeutic agent, irradiation or any combination thereof, said method comprises the steps of:
a. determining the level of expression of at least one APOBEC family member in at least one biological sample of said subject, to obtain an expression value;
b. determining if the expression value obtained in step (a) is any one of, positive or negative with respect to a predetermined standard expression value or to an expression value of said APOBEC family member in a control sample;
Wherein a negative expression value of said APOBEC family member indicates that said subject responds to said genotoxic treatment.

33. The method according to claim 32, for determining a genotoxic treatment regimen for a subject suffering from a proliferative disorder, said method comprises the steps of:
a. determining the level of expression of at least one APOBEC family member in at least one biological sample of said subject, to obtain an expression value;
b. determining if the expression value obtained in step (a) is any one of, positive or negative with respect to a predetermined standard expression value or to an expression value of APOBEC family member in a control sample;
Wherein a positive expression value of said APOBEC family member indicates that at least one compound that inhibits the expression or the activity of said APOBEC family member is required in addition to said genotoxic treatment for said subject.

34. (canceled)


SEQUENCE LISTING (92 sequences)

SEQ ID NO: 1 - protein, 15 aa, Artificial Sequence
  Viral - Vif peptide
MENRWQVMIVWQVDR

SEQ ID NO: 2 - protein, 15 aa, Artificial Sequence
  Viral - Vif peptide
WQVMIVWQVDRMRIR

SEQ ID NO: 3 - protein, 15 aa, Artificial Sequence
  Viral - Vif peptide
IVWQVDRMRIRTWKS

SEQ ID NO: 4 - protein, 15 aa, Artificial Sequence
  Viral - Vif peptide
VDRMRIRTWKSLVKH

SEQ ID NO: 5 - protein, 15 aa, Artificial Sequence
  Viral - Vif peptide
RIRTWKSLVKHHMYI

SEQ ID NO: 6 - protein, 15 aa, Artificial Sequence
  Viral - Vif peptide
WKSLVKHHMYISGKA

SEQ ID NO: 7 - protein, 15 aa, Artificial Sequence
  Viral - Vif peptide
VKHHMYISGKAKGWF

SEQ ID NO: 8 - protein, 15 aa, Artificial Sequence
  Viral - Vif peptide
MYISGKAKGWFYRHH

SEQ ID NO: 9 - protein, 15 aa, Artificial Sequence
  Viral - Vif peptide
GKAKGWFYRHHYEST

SEQ ID NO: 10 - protein, 15 aa, Artificial Sequence
  Viral - Vif peptide
GWFYRHHYESTHPRI

SEQ ID NO: 11 - protein, 15 aa, Artificial Sequence
  Viral - Vif peptide
RHHYESTHPRISSEV

SEQ ID NO: 12 - protein, 15 aa, Artificial Sequence
  Viral - Vif peptide
ESTHPRISSEVHIPL

SEQ ID NO: 13 - protein, 15 aa, Artificial Sequence
  Viral - Vif peptide
PRISSEVHIPLGDAR

SEQ ID NO: 14 - protein, 15 aa, Artificial Sequence
  Viral - Vif peptide
SEVHIPLGDARLVIT

SEQ ID NO: 15 - protein, 15 aa, Artificial Sequence
  Viral - Vif peptide
IPLGDARLVITTYWG

SEQ ID NO: 16 - protein, 15 aa, Artificial Sequence
  Viral - Vif peptide
DARLVITTYWGLHTG

SEQ ID NO: 17 - protein, 15 aa, Artificial Sequence
  Viral - Vif peptide
VITTYWGLHTGERDW

SEQ ID NO: 18 - protein, 15 aa, Artificial Sequence
  Viral - Vif peptide
YWGLHTGERDWHLGQ

SEQ ID NO: 19 - protein, 15 aa, Artificial Sequence
  Viral - Vif peptide
HTGERDWHLGQGVSI

SEQ ID NO: 20 - protein, 15 aa, Artificial Sequence
  Viral - Vif peptide
RDWHLGQGVSIEWRK

SEQ ID NO: 21 - protein, 15 aa, Artificial Sequence
  Viral - Vif peptide
LGQGVSIEWRKKRYS

SEQ ID NO: 22 - protein, 15 aa, Artificial Sequence
  Viral - Vif peptide
VSIEWRKKRYSTQVD

SEQ ID NO: 23 - protein, 15 aa, Artificial Sequence
  Viral - Vif peptide
WRKKRYSTQVDPDLA

SEQ ID NO: 24 - protein, 15 aa, Artificial Sequence
  Viral - Vif peptide
RYSTQVDPDLADQLI

SEQ ID NO: 25 - protein, 15 aa, Artificial Sequence
  Viral - Vif peptide
QVDPDLADQLIHLYY

SEQ ID NO: 26 - protein, 15 aa, Artificial Sequence
  Viral - Vif peptide
DLADQLIHLYYFDCF

SEQ ID NO: 27 - protein, 15 aa, Artificial Sequence
  Viral - Vif peptide
QLIHLYYFDCFSESA

SEQ ID NO: 28 - protein, 15 aa, Artificial Sequence
  Viral - Vif peptide
LYYFDCFSESAIRNA

SEQ ID NO: 29 - protein, 15 aa, Artificial Sequence
  Viral - Vif peptide
DCFSESAIRNAILGH

SEQ ID NO: 30 - protein, 15 aa, Artificial Sequence
  Viral - Vif peptide
ESAIRNAILGHIVSP

SEQ ID NO: 31 - protein, 15 aa, Artificial Sequence
  Viral - Vif peptide
RNAILGHIVSPRCEY

SEQ ID NO: 32 - protein, 15 aa, Artificial Sequence
  Viral - Vif peptide
LGHIVSPRCEYQAGH

SEQ ID NO: 33 - protein, 15 aa, Artificial Sequence
  Viral - Vif peptide
VSPRCEYQAGHNKVG

SEQ ID NO: 34 - protein, 15 aa, Artificial Sequence
  Viral - Vif peptide
CEYQAGHNKVGSLQY

SEQ ID NO: 35 - protein, 15 aa, Artificial Sequence
  Viral - Vif peptide
AGHNKVGSLQYLALA

SEQ ID NO: 36 - protein, 15 aa, Artificial Sequence
  Viral - Vif peptide
KVGSLQYLALAALIT

SEQ ID NO: 37 - protein, 15 aa, Artificial Sequence
  Viral - Vif peptide
LQYLALAALITPKKI

SEQ ID NO: 38 - protein, 15 aa, Artificial Sequence
  Viral - Vif peptide
ALAALITPKKIKPPL

SEQ ID NO: 39 - protein, 15 aa, Artificial Sequence
  Viral - Vif peptide
LITPKKIKPPLPSVT

SEQ ID NO: 40 - protein, 15 aa, Artificial Sequence
  Viral - Vif peptide
KKIKPPLPSVTKLTE

SEQ ID NO: 41 - protein, 15 aa, Artificial Sequence
  Viral - Vif peptide
PPLPSVTKLTEDRWN

SEQ ID NO: 42 - protein, 15 aa, Artificial Sequence
  Viral - Vif peptide
SVTKLTEDRWNKPQK

SEQ ID NO: 43 - protein, 15 aa, Artificial Sequence
  Viral - Vif peptide
LTEDRWNKPQKTKGH

SEQ ID NO: 44 - protein, 15 aa, Artificial Sequence
  Viral - Vif peptide
RWNKPQKTKGHRGSH

SEQ ID NO: 45 - protein, 15 aa, Artificial Sequence
  Viral - Vif peptide
PQKTKGHRGSHTMNG

SEQ ID NO: 46 - protein, 12 aa, Artificial Sequence
  Viral - Vif peptide
KGHRGSHTMNGH

SEQ ID NO: 47 - DNA, 80 bp, Artificial Sequence
  80-mer ss-DNA
ggattggttggttatttgtttaaggaaggtggattaaaggcccaataaggtgatggaagttatgtttggtagattgatgg

SEQ ID NO: 48 - DNA, 26 bp, Artificial Sequence
  Primer 5′ to 3′ forward
ggattggttggttatttgtttaagga

SEQ ID NO: 49 - DNA, 27 bp, Artificial Sequence
  Primer 5′ to 3′ reverse
ccatcaatctaccaaacataacttcca

SEQ ID NO: 50 - protein, 192 aa, Human immunodeficiency virus
MENRWQVMIVWQVDRMRIRTWKSLVKHHMYISGKAKGWFYRHHYESTHPRISSEVHIPLGDARLVITTYWGLHTGERDWHLGQGVSIEWRKRRYSTQVDPDLADQLIHLYYFDCFSESAIRNAILGHIVSPSCEYQAGHNKVGSLQYLALAALITPKKIKPPLPSVTKLTEDRWNKPQKTKGHRGSHTMNGH

SEQ ID NO: 51 - protein, 51 aa, Artificial Sequence
  Viral - Vif peptide
MENRWQVMIVWQVDRMRIRTWKSLVKHHMYISGKAKGWFYRHHYESTHPRI

SEQ ID NO: 52 - protein, 27 aa, Artificial Sequence
  Viral - Vif peptide
DLADQLIHLYYFDCFSESAIRNAILGH

SEQ ID NO: 53 - DNA, 160 bp, Artificial Sequence
  S160
ggattggttggttatttgtttaaggaaggaagaaggaaggcccaataagggaaggcccaataagtgggaaggcccaataagtgaattaaataagtgattgataaataagtgattgataagttgattaaatagttggaagttatgtttggtagattgatgg

SEQ ID NO: 54 - DNA, 30 bp, Artificial Sequence
  Complementary to 5′
accttccttaaacaaataaccaaccaatcc

SEQ ID NO: 55 - DNA, 30 bp, Artificial Sequence
  Complementary to 3′
ccatcaatctaccaaacataacttccatca

SEQ ID NO: 56 - DNA, 79 bp, Artificial Sequence
  DNA1 - biotin
ggttaggaggatattgcgttgagatattgaatgatgaaccctaatgagtcgtattgaaagagagagtgtagagtggccc

SEQ ID NO: 57 - DNA, 79 bp, Artificial Sequence
  DNA2
ggattggttggttatttgtttaaggaaggtggattaaaggcccataaggtgatggaagttatgtttggtagattgatgg

SEQ ID NO: 58 - DNA, 79 bp, Artificial Sequence; identical to SEQ ID NO: 57
  SC sequence

SEQ ID NO: 59 - DNA, 60 bp, Artificial Sequence
  SU3-R
catataagcagctgctttttgcctgtactgggtctctctggttagaccagatctgagcct

SEQ ID NO: 60 - DNA, 46 bp, Artificial Sequence
  gDNA
caatatcatacgccgagagccgggtctctctggttagaccagaaaa

SEQ ID NO: 61 - DNA, 19 bp, Artificial Sequence
  pF509
acccactgcttaagcctca

SEQ ID NO: 62 - DNA, 20 bp, Artificial Sequence
  pR604
gtctgagggatctctagtta

SEQ ID NO: 63 - DNA, 20 bp, Artificial Sequence
  pFTag
caatatcatacgccgagagc

SEQ ID NO: 64 - DNA, 51 bp, Artificial Sequence
  S51CCC biotin
tagttagccagagagctcccaggctcagatctggtctaaccagagagaccc

SEQ ID NO: 65 - DNA, 51 bp, Artificial Sequence
  S51CUU biotin
tagttagccagagagctcccaggctcagatctggtctaaccagagagacuu

SEQ ID NO: 66 - protein, 4 aa, Artificial Sequence
  Peptide concensus
VKHH

SEQ ID NO: 67 - protein, 5 aa, Artificial Sequence
  Peptide consensus
XLYYF

SEQ ID NO: 68 - protein, 4 aa, Artificial Sequence
  Peptide consensus
KGWF

SEQ ID NO: 69 - protein, 8 aa, Artificial Sequence
  Viral - Vif peptide
SLVKHHMY

SEQ ID NO: 70 - protein, 8 aa, Homo sapiens
VVKHHSPV

SEQ ID NO: 71 - protein, 9 aa, Artificial Sequence
  Viral - Vif peptide
IHLYYFDCF

SEQ ID NO: 72 - protein, 6 aa, Artificial Sequence
  Viral - Vif peptide
HLYYFD

SEQ ID NO: 73 - protein, 5 aa, Artificial Sequence
  Viral - Vif peptide
HLYYF

SEQ ID NO: 74 - protein, 9 aa, Homo sapiens
ARLYYFWDT

SEQ ID NO: 75 - protein, 7 aa, Homo sapiens
RLYYFWD

SEQ ID NO: 76 - protein, 10 aa, Artificial Sequence
  Viral - Vif peptide
YISGKAKGWF

SEQ ID NO: 77 - protein, 10 aa, Artificial Sequence
  Viral - Vif peptide
VKHHMYISGK

SEQ ID NO: 78 - protein, 7 aa, Artificial Sequence
  Viral - Vif peptide
GKAKGWF

SEQ ID NO: 79 - protein, 7 aa, Artificial Sequence
  Viral - Vif peptide
LYYFDCF

SEQ ID NO: 80 - protein, 9 aa, Artificial Sequence
  Viral - Vif peptide
LYYFDCFSE

SEQ ID NO: 81 - protein, 384 aa, Artificial Sequence
  A3G W285A mutant
MKPHFRNTVERMYRDTFSYNFYNRPILSRRNTVWLCYEVKTKGPSRPPLDAKIFRGQVYSELKYHPEMRFFHWFSKWRKLHRDQEYEVTWYISWSPCTKCTRDMATFLAEDPKVTLTIFVARLYYFWDPDYQEALRSLCQKRDGPRATMKIMNYDEFQHCWSKFVYSQRELFEPWNNLPKYYILLHIMLGEILRHSMDPPTFTFNFNNEPWVRGRHETYLCYEVERMHNDTWVLLNQRRGFLCNQAPHKHGFLEGRHAELCFLDVIPFWKLDLDQDYRVTCFTSASPCFSCAQEMAKFISKNKHVSLCIFTARIYDDQGRCQEGLRTLAEAGAKISIMTYSEFKHCWDTFVDHQGCPFQPWDGLDEHSQDLSGRLRAILQNQEN

SEQ ID NO: 82 - protein, 4 aa, Artificial Sequence
  Viral - Vif peptide
LYYF

SEQ ID NO: 83 - protein, 15 aa, Homo sapiens
WVRGRHETYLCYEVE

SEQ ID NO: 84 - protein, 15 aa, Homo sapiens
RMHNDTWVLLNQRRG

SEQ ID NO: 85 - protein, 6 aa, Homo sapiens
RMHNDT

SEQ ID NO: 86 - protein, 30 aa, Homo sapiens
WVRGRHETYLCYEVERMHNDTWVLLNQRRG

SEQ ID NO: 87 - protein, 10 aa, Artificial Sequence
  Viral - Vif peptide
SAIRNAILGH

SEQ ID NO: 88 - protein, 384 aa, Homo sapiens
MKPHFRNTVERMYRDTFSYNFYNRPILSRRNTVWLCYEVKTKGPSRPPLDAKIFRGQVYSELKYHPEMRFFHWFSKWRKLHRDQEYEVTWYISWSPCTKCTRDMATFLAEDPKVTLTIFVARLYYFWDPDYQEALRSLCQKRDGPRATMKIMNYDEFQHCWSKFVYSQRELFEPWNNLPKYYILLHIMLGEILRHSMDPPTFTFNFNNEPWVRGRHETYLCYEVERMHNDTWVLLNQRRGFLCNQAPHKHGFLEGRHAELCFLDVIPFWKLDLDQDYRVTCFTSWSPCFSCAQEMAKFISKNKHVSLCIFTARIYDDQGRCQEGLRTLAEAGAKISIMTYSEFKHCWDTFVDHQGCPFQPWDGLDEHSQDLSGRLRAILQNQEN

SEQ ID NO: 89 - protein, 373 aa, Homo sapiens
MKPHFRNTVERMYRDTFSYNFYNRPILSRRNTVWLCYEVKTKGPSRPRLDAKIFRGQVYSQPEHHAEMCFLSWFCGNQLPAYKCFQITWFVSWTPCPDCVAKLAEFLAEHPNVTLTISAARLYYYWERDYRRALCRLSQAGARVKIMDDEEFAYCWENFVYSEGQPFMPWYKFDDNYAFLHRTLKEILRNPMEAMYPHIFYFHFKNLRKAYGRNESWLCFTMEVVKHHSPVSWKRGVFRNQVDPETHCHAERCFLSWFCDDILSPNTNYEVTWYTSWSPCPECAGEVAEFLARHSNVNLTIFTARLYYFWDTDYQEGLRSLSQEGASVEIMGYKDFKYCWENFVYNDDEPFKPWKGLKYNFLFLDSKLQEILE

SEQ ID NO: 90 - protein, 384 aa, Artificial Sequence
  A3G E259Q mutant
MKPHFRNTVERMYRDTFSYNFYNRPILSRRNTVWLCYEVKTKGPSRPPLDAKIFRGQVYSELKYHPEMRFFHWFSKWRKLHRDQEYEVTWYISWSPCTKCTRDMATFLAEDPKVTLTIFVARLYYFWDPDYQEALRSLCQKRDGPRATMKIMNYDEFQHCWSKFVYSQRELFEPWNNLPKYYILLHIMLGEILRHSMDPPTFTFNFNNEPWVRGRHETYLCYEVERMHNDTWVLLNQRRGFLCNQAPHKHGFLEGRHAQLCFLDVIPFWKLDLDQDYRVTCFTSWSPCFSCAQEMAKFISKNKHVSLCIFTARIYDDQGRCQEGLRTLAEAGAKISIMTYSEFKHCWDTFVDHQGCPFQPWDGLDEHSQDLSGRLRAILQNQEN

SEQ ID NO: 91 - protein, 616 aa, Homo sapiens
MVGQLSEGAIAAIMQKGDTNIKPILQVINIRPITTGNSPPRYRLLMSDGLNTLSSFMLATQLNPLVEEEQLSSNCVCQIHRFIVNTLKDGRRVVILMELEVLKSAEAVGVKIGNPVPYNEGLGQPQVAPPAPAASPAASSRPQPQNGSSGMGSTVSKAYGASKTFGKAAGPSLSHTSGGTQSKVVPIASLTPYQSKWTICARVTNKSQIRTWSNSRGEGKLFSLELVDESGEIRATAFNEQVDKFFPLIEVNKVYYFSKGTLKIANKQFTAVKNDYEMTFNNETSVMPCEDDHHLPTVQFDFTGIDDLENKSKDSLVDIIGICKSYEDATKITVRSNNREVAKRNIYLMDTSGKVVTATLWGEDADKFDGSRQPVLAIKGARVSDFGGRSLSVLSSSTIIANPDIPEAYKLRGWFDAEGQALDGVSISDLKSGGVGGSNTNWKTLYEVKSENLGQGDKPDYFSSVATVVYLRKENCMYQACPTQDCNKKVIDQQNGLYRCEKCDTEFPNFKYRMILSVNIADFQENQWVTCFQESAEAILGQNAAYLGELKDKNEQAFEEVFQNANFRSFIFRVRVKVETYNDESRIKATVMDVKPVDYREYGRRLVMSIRRSALM

SEQ ID NO: 92 - DNA, 58 bp, Artificial Sequence
  A3G shRNA
ccgggccaggtgtattccgaacttactcgagtaagttcggaatacacctggctttttg